(19) United States
(12) Patent Application Publication
(10) Pub. No.: US 2026/0146259 A1
(43) Pub. Date: May 28, 2026

(54) COMPOSITIONS AND SYSTMES FOR HERPES SIMPLEX VIRUS PACKAGING AND USES THEREOF

(71) Applicants: Opentrons Labworks Inc., Long Island City, NY (US); University of Pittsburgh - Of the Commonwealth System of Higher Education, Pittsburgh, PA (US)

(72) Inventors: Leslie MITCHELL, New York, NY (US); Megan HOGAN, New York, NY (US); Thomas DESMARAIS, New York, NY (US); Joseph C. GLORIOSO, III, Cambridge, MA (US); William F. GOINS, Pittsburgh, PA (US); Selene INGUSCI, Pittsburgh, PA (US); Justus Bernard COHEN, Allison Park, PA (US)

(21) Appl. No.: 19/121,300

(22) PCT Filed: Oct. 20, 2023
(86) PCT No.: PCT/US2023/077467

§ 371 Date: Apr. 15, 2025

Related U.S. Application Data
(60) Provisional application No. 63/380,315, filed on Oct. 20, 2022.

(51) Int. Cl. C12N 15/86; C12N 7/02; C12N 9/22; C12N 15/113

(52) U.S. Cl. CPC C12N 15/86; C12N 7/025; C12N 9/226; C12N 15/113; C12N 2310/20; C12N 2710/16652; C12N 2800/107; C12N 2830/003; C12N 2830/34; C12N 2830/36

(57) ABSTRACT

Provided herein are compositions for a Herpes Simplex Virus packaging system and packaging cell line. Also provided herein are compositions, systems, and methods for producing a packaging cell line for Herpes Simplex Virus amplicons.

### CROSS REFERENCE

This application claims the benefit of U.S. Provisional Application No. 63/380,315, filed on Oct. 20, 2022, which is incorporated herein by reference in its entirety.

### STATEMENT AS TO FEDERALLY SPONSORED RESEARCH

This invention was made with the support of the United States government under Grant No. 1R43TR003558-01A1 awarded by the Small Business Innovation Research (SBIR). The government has certain rights in the invention.

### SEQUENCE LISTING

This instant application contains a Sequence Listing which has been submitted electronically in XML format and is hereby incorporated by reference in its entirety. Said XML copy, created on Oct. 18, 2023, is named 59725-714_601_SL.xml and is 1,102,159 bytes in size.

### BACKGROUND

Gene therapy has been rapidly developing as a treatment strategy for diseases affecting a variety of distinct metabolic, neurologic and cancer-related conditions. The challenge of gene therapy remains the creation of gene delivery vehicles that provide novel genetic information to different cell and tissue types and express the desired gene of interest at appropriate levels and duration. The gene therapy field has endeavored to take advantage of viral systems engineered to remove viral replication and cytotoxic functions. The bar for viral vectors is high since they must be safe and nonpathogenic, amenable to consistent manufacture, and capable of delivering a therapeutic payload to the correct tissue and cell type. The therapeutic gene may need to be expressed not only at the appropriate level, and thus be regulated either by transcriptional or post-transcriptional mechanisms, but also for an appropriate length of time in order to achieve maximal benefit. In addition, the therapy may need to persist without damage to the transduced cells or recruitment of immune responses that could eliminate corrected cells and shut down transgene expression. Furthermore, production of viral vectors with sufficient quantity and quality (e.g., purity) poses another challenge to the practice of gene therapy. Therefore, there is a desire for improved vector systems and packaging cell lines.

### SUMMARY

In some aspects, provided herein is a packaging cell line comprising a recombinant genome, wherein the recombinant genome comprises a. a first nucleic acid sequence comprising one or more Herpes Simplex Virus (HSV) Class I genes; and b. a second nucleic acid sequence comprising one or more HSV Class I and Class II genes.

In some aspects, provided herein is a composition comprising a recombinant genome, wherein the recombinant genome comprises a. a first nucleic acid sequence comprising one or more Herpes Simplex Virus (HSV) Class I genes; and b. a second nucleic acid sequence comprising one or more HSV Class I and Class II genes.

In some aspects, provided herein is a method of producing a packaging cell line comprising introducing into a cell comprising a genome: a. a first nucleic acid sequence comprising one or more Herpes Simplex Virus (HSV) Class I genes; and b. a second nucleic acid sequence comprising one or more HSV Class I and Class II genes.

In some embodiments, the first nucleic acid sequence or the second nucleic acid sequence may comprise one or more regulatory elements that are functionally connected to one or more HSV Class I genes or HSV Class II genes. These one or more regulatory elements may comprise, in some embodiments, an enhancer, a promoter, a terminator, or a combination thereof. For example, the one or more regulatory elements may comprise a promoter comprising a natural (or native) promoter of HSV Class I genes or HSV Class II genes, a conditional promoter, or an inducible promoter (e.g., Tet-On promoter). An inducible promoter can be useful for controlling and/or managing the expression of a gene (e.g., HSV Class I or HSV Class II genes) that may be toxic to host cells or packaging cell lines or a gene for which constitutive expression is not required (e.g., transient expression at a specific time is needed). In some embodiments, the first nucleic acid sequence, the second nucleic acid sequence, or the recombinant genome of the packaging cell line may not comprise an HSV origin of replication (ori), an HSV packaging signal (pac), or a combination thereof.

In some embodiments, the first nucleic acid sequence or the second nucleic acid sequence may be introduced to a first genomic safe harbor site of the recombinant genome comprising a human ortholog of the mouse Rosa26 locus (or Rosa26 homolog locus). In some embodiments, the first nucleic acid sequence or the second nucleic acid sequence may be introduced to a second genomic safe harbor site of the recombinant genome comprising an adeno-associated virus site 1 (AAVS1) locus.

In some aspects, provided herein, is a system for producing a Herpes Simplex Virus (HSV) amplicon particle comprising a. any packaging cell line descried herein; and b. an amplicon vector. In some embodiments, the amplicon vector may not comprise the one or more HSV Class I genes or the one or more HSV Class II genes introduced to the packing cell line described herein. Accordingly, in some embodiments, the amplicon vector can comprise a cargo nucleic acid sequence comprising up to about 150 kb. In some embodiments, the amplicon vector may comprise an HSV origin of replication (ori), an HSV packaging signal (pac), or a combination thereof. In some embodiments, the HSV amplicon particle produced and isolated from the packaging cell line can be re-introduced to the packing cell line for high titer HSV amplicon particle production.

### INCORPORATION BY REFERENCE

Each patent, publication, and non-patent literature cited in the application is hereby incorporated by reference in its entirety as if each was incorporated by reference individually. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “Figure” and “FIG.” herein), of which:

FIGS. 1A-1C show a schematic representation of various components of the present disclosure. FIG. 1A shows that an HSV amplicon vector can accommodate up to about 150 kb of payload DNA for delivery to cells. The HSV amplicon vector also comprises HSV origin of replication (ori) and packaging (pac) sequences, as well as HSV Class I gene subset A and HSV Class II gene subset A. FIG. 1B shows that the HSV amplicon vector can be introduced into an HSV packaging cell line by transfection. The HSV packaging cell line has HSV Class I genes and HSV Class II genes inserted at 2 distinct genomic safe harbor (GSH) sites on different chromosomes, denoted as GSH 1 (yellow) and GSH 2 (orange). HSV Class I genes can be inserted at GSH 1, while HSV Class II genes can be inserted at GSH 2. FIG. 1C shows that when the HSV amplicon vector is introduced into the HSV packaging cell line. HSV amplicon particles can be produced and isolated for DNA delivery applications. Such HSV amplicon particles can be used for re-passaging to increase titer.

FIGS. 2A and 2B show insertion of UL19, UL20, and UL21 at a REWRITE landing pad located at the AAVS1 locus (GSH2). FIG. 2A shows a schematic drawing of a gene cassette containing HSV class II genes (UL19, UL20, and UL21) integrated at GSH2. FIG. 2B shows an exemplary readout of whole-genome sequencing (WGS) after enrichment for sequences (Capture-seq) at the AAVS1 genome and sequences contained in the synHSV93 genome in U2OS-4/27+UL19-21 cells.

FIGS. 3A and 3B show complementation of ICP4 (IE175 gene) and ICP27 (UL54 gene) in U2OS-4/27+UL19-21 cells. FIG. 3A shows representative fluorescent images of JDNI8-4 plaques for each cell line (U2OS-4/27 cells, U2OS+REWRITE Landing Pad cells, U2OS-4/27+UL19-21 pooled clones and individual clones of U2OS-4/27+UL19-21) at 5 days post-infection (dpi). FIG. 3B shows viral titers for individual clones normalized to U2OS-4/27.

FIG. 4 shows complementation of VP5 (UL19 gene) in U2OS-4/27+UL19-21 cells by infection with a replication competent virus deleted for UL19 gene (ΔVP5; HSV-KOS deleted for VP5/UL19 gene).

FIGS. 5A and 5B show comparison of ΔVP5 HSV virus pfu titer in U2OS-4/27+UL19-21 clones and G5 (packaging cell line stably transduced to express UL16-UL21 HSV genes, used to complement ΔVP5 virus). FIG. 5A shows representative images of plaques formed by each cell line with 10⁻⁶ dilution of ΔVP5 HSV virus at 7 dpi. G5 and U2OS-4/27+UL19-21 clones were infected with serial dilution of dVP5. U2OS-4/27 and Vero cells were infected similarly (negative controls). FIG. 5B shows titer (pfu/ul) for each cell line with ΔVP5 HSV virus. Titer was determined by plaque count at 7 dpi.

FIGS. 6A and 6B show complementation of VP5 (UL19 gene), UL20p (UL20 gene) and UL21 (UL21 gene) in U2OS-4/27+UL19-21 clones. FIG. 6A shows that U2OS-4/27+UL19-21 clones complement replication deficient mutant HSV vector JDNI8-AUL19-UL21 (JDNI8 vector deleted additionally for UL19-UL21 genes). FIG. 6B shows propagation of the JDN18-ΔUL19-UL21 vector on U2OS-4/27+UL19-21 clones.

FIGS. 7A and 7B show insertion of synHSV128 genome at a REWRITE landing pad located at the AAVS1 locus (GSH2). FIG. 7A shows a schematic drawing of a synthetic genome integrated at GSH2. FIG. 7B shows an exemplary readout of whole-genome sequencing (WGS) after enrichment for sequences (Capture-seq) at the AAVS1 genome and sequences contained in the synHSV128 genome in U2OS+synHSV128.

FIGS. 8A and 8B show complementation of replication competent HSV viruses with synHSV128 packaging cells. FIG. 8A shows complementation of a ΔVP5 replication competent virus (ΔVP5; HSV-KOS deleted for VP5 gene) in U2OS+synHSV128 packaging cells. FIG. 8B shows complementation of a ΔgD (US6 gene) replication competent virus (ΔgD; HSV-KOS deleted for gD/US6 gene) in Vero VD60 (ΔgD virus packaging cell line stably transduced to express gD/US6 gene), U2OS-synHSV128, and Vero cells (negative control).

### DETAILED DESCRIPTION

Provided herein are compositions and systems for a packaging cell line comprising a recombinant genome, wherein the recombinant genome comprises a nucleic acid sequence comprising one or more Herpes Simplex Virus (HSV) Class I genes and/or a nucleic acid sequence comprising one or more HSV Class I and Class II genes. Also provided herein are methods for producing packaging cell lines described herein.

### Definitions

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. The terms “and/or,” “a combination thereof,” and “any combination thereof” and their grammatical equivalents as used herein, can be used interchangeably. These terms can convey that any combination is specifically contemplated. Solely for illustrative purposes, the following phrases “A, B, and/or C,” “A, B, C, or a combination thereof,” or “A, B. C, or any combination thereof” can mean “A individually; B individually; C individually; A and B; B and C; A and C; and A, B, and C.” The term “or” can be used conjunctively or disjunctively, unless the context specifically refers to a disjunctive use.

The term “about” or “approximately” can mean within an acceptable error range for the particular value, which may depend in part on how the value is measured or determined, e.g., the limitations of the measurement system. For example, “about” can mean within 1 or more than 1 standard deviation. Alternatively, “about” can mean a range of up to 20%, up to 10%, up to 5%, or up to 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value should be assumed.

Throughout this disclosure, numerical features are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the present disclosure, subject to any specifically excluded limit in the stated range. Where the stated range includes one or both of the limits, ranges excluding either or both of those included limits are also included in the present disclosure, unless the context clearly dictates otherwise.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

Reference in the specification to “embodiments,” “certain embodiments,” “preferred embodiments,” “specific embodiments,” “some embodiments,” “an embodiment,” “one embodiment” or “other embodiments” means that a particular feature, structure, or characteristic described in connection with the embodiments is included in at least some embodiments, but not necessarily all embodiments, of the present disclosures. To facilitate an understanding of the present disclosure, a number of terms and phrases are defined below.

Certain specific details of this description are set forth in order to provide a thorough understanding of various embodiments. However, one skilled in the art will understand that the present disclosure may be practiced without these details. In other instances, well-known techniques or methods have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed disclosure.

The terms “nucleic acid sequence,” “polynucleic acid sequence,” and/or “nucleotide sequence” are used herein interchangeably and have the identical meaning herein and refer to DNA or RNA. In some embodiments, a nucleic acid sequence is a polymer comprising or consisting of nucleotide monomers, which are covalently linked to each other by phosphodiester-bonds of a sugar/phosphate-backbone. The terms “nucleic acid sequence,” “polynucleic acid sequence,” and “nucleotide sequence” may encompass unmodified nucleic acid sequences, i.e., comprise unmodified nucleotides, or natural nucleotides. In some embodiments, “natural nucleotide,” “unmodified nucleotide.” and/or “canonical nucleotide” are used herein interchangeably and have the identical meaning herein and refer to the naturally occurring nucleotide bases adenine (A), guanine (G), cytosine (C), uracil (U), and/or thymine (T). The terms “nucleic acid sequence,” “polynucleic acid sequence,” and “nucleotide sequence” may also encompass modified nucleic acid sequences, such as base-modified, sugar-modified or backbone-modified etc., DNA or RNA.

Although methods and materials similar or equivalent to those described herein can be used in the practice or testing of the present disclosure, suitable methods, and materials are described below.

### Herpes Simplex Virus (HSV) and HSV Vectors

Herpes Simplex Virus (HSV) is an enveloped virus containing a double stranded DNA genome that is over 150 kb. As used herein. HSV may be HSV-1 or HSV-2. In some embodiments, the HSV may be HSV-1. In some embodiments, the HSV may be HSV-2. The linear double-stranded DNA HSV genome is divided into a unique long (UL) region and a unique short (US) region, which are bounded by regions of inverted repeats, termed internal repeats, i.e., two copies of a long inverted repeat (RL), and two copies of a short inverted repeat (RS). The genome termini are bounded by regions of direct repeats, termed terminal repeats (TR). For example, the core of the HSV-1 virion contains a 152-kb double-stranded linear DNA genome, which encodes 80-85 viral genes in the UL and US regions. The inverted repeat sequences flanking the UL and US regions comprise sequences required for cleavage/packaging of the HSV-1 genome.

HSV genes are expressed in waves, referred to as “cascade regulation,” producing first the immediate early (IE), then the early (E), and finally the late (L) gene products. The gene program is launched by a component of the infecting particle, the viral tegument protein VP16 delivered to the cell nucleus during infection that induces the expression of IE genes through binding to a viral enhancer sequence present in multiple copies within the IE gene promoters. The IE gene products have multiple functions including transactivation of the early genes, mRNA transport, and interfering with innate host immune responses that include STING activation and IFN production. The early gene products are primarily responsible for viral genome replication whereupon the late genes are expressed that encode primarily structural proteins needed for DNA packaging and particle assembly. As used herein, HSV Class I genes may comprise IE genes. In some embodiments, HSV class I genes may comprise ICP4, ICP27, ICP0, ICP22. ICP34.5. VP16, or a combination thereof. HSV Class II genes may comprise early genes, including but not limited to UL1, UL2, UL3, UL4, UL5, UL6, UL7, UL8, UL9, UL10, UL 11, UL12, UL13, UL14, UL15, UL16, UL17, UL18, UL19, UL20, UL21, UL22, UL23, UL24, UL25, UL26, UL26.5, UL27, UL28, UL29, UL30, UL31, UL32, UL33, UL34, UL35, UL36, UL37, UL38, UL39, UL40, UL41, UL42, UL43, UL44, UL45, UL46, UL47, UL49, UL49A, UL50, UL51, UL52, UL53, UL55, UL56, US1, US2, US3, US4, USS. US6, US7, US8. US9, US10, US11, and US12.

Many of the HSV genes are not essential for virus production per se but rather are important to carry out the complex life cycle in the host in vivo. HSV infects host cells using an attachment/fusion entry mechanism minimally requiring 5 envelope glycoproteins (gB, gC, gD, gH/gL) and virus production occurs in the host cell nucleus. Virus replication is lytic and newly formed particles acquire their envelope through nuclear membrane budding, Golgi processing, and envelope exchange at the cell surface. HSV in the latent phase can persist for life in sensory neurons of the peripheral nervous system, and in brain if infected. The viral genomes are maintained as episomes and their copy number can be high. During latency, viral lytic genes are largely silenced and only the noncoding latency associated transcript (LAT) gene is expressed. The virus is capable of reactivation from latency, which provides a means of virus spread to other individuals by direct contact with a recurrent lesion. Like HSV genomes in latency, replication-defective HSV and amplicon genomes remain extrachromosomal, but unlike latent HSV, in a cell-type independent manner. HSV vector technologies (including sequence information) are described in U.S. Pat. Nos. 5,658,724, 5,804,413, 5,849,571, 5,849,572, 5,879,934, 5,998,174, 6,261,552, 7,078,029, 7,531,167, 10,174,341, and 10,201,575.

As used herein. HSV vectors can include replication defective (or deficient) HSV vectors and HSV amplicon vectors. As used herein, the replication defective (or deficient) HSV vectors can refer to HSV vectors where “essential” genes (e.g., Class I genes and/or Class II genes) are muted, deleted, or replaced with transgenes intended for long-term expression from the otherwise quiescent viral episomal genome. In some embodiments of the present disclosure, HSV vectors may comprise a mutation or deletion of Class I genes. e.g., mutation or deletion of ICP4, ICP27, ICP0, ICP22, ICP34.5, VP16, or a combination thereof. Additionally or alternatively, in some embodiments, HSV vectors may comprise a mutation or deletion of Class II genes, e.g., mutation or deletion of UL1, UL2, UL3, UL4, UL5, UL6, UL7, UL8, UL9, UL10, UL 11, UL12, UL13, UL14, UL15, UL16, UL17, UL18, UL19, UL20, UL21, UL22, UL23, UL24, UL25, UL26, UL26.5, UL27, UL28, UL29, UL30, UL31, UL32, UL33, UL34, UL35, UL36, UL37, UL38, UL39, UL40, UL41, UL42, UL43, UL44, UL45, UL46, UL47, UL49, UL49A, UL50, UL51, UL52, UL53, UL55, UL56, US1, US2, US3, US4, US5, US6, US7, US8, US9, US10, US11, US12, or a combination thereof. In some embodiments. HSV vectors may comprise a mutation in or deletion of UL19, UL20, UL21, or a combination thereof. In some embodiments, HSV vectors may comprise a mutation in or deletion of UL19, UL20, and UL21. In some embodiments, HSV vectors may comprise a mutation in or deletion of UL19, UL20, UL21, UL41, or a combination thereof. In some embodiments, HSV vectors may comprise a mutation in or deletion of UL19, UL20, UL21, and UL41.

The amplicon vector of the present technology may be an amplicon plasmid comprising ori/pac sequences and a plasmid backbone, or minicircle DNA comprising ori/pac sequences. Each of the amplicon plasmid and minicircle additionally may comprise at least one cargo sequence or payload comprising a transgene. Therefore, the amplicon vector may comprise ori/pac sequences and at least one cargo sequence comprising a transgene. In some embodiments, the amplicon vector may additionally comprise one or more HSV Class II genes. In some embodiments, the amplicon vector may additionally comprise one or more HSV Class I genes. In some embodiments, an HSV amplicon may further comprise a bacterial origin of DNA replication and an antibiotic resistance gene for propagation in bacteria. In some embodiments, a bacterial origin of DNA replication may comprise ColE1. In some embodiments, an antibiotic resistance gene may include Ampicillin resistance.

The plasmid backbone in the amplicon vector may comprise a bacterial artificial chromosome (BAC) cassette. The inclusion of such BAC cassette facilitates propagation and manipulation of the amplicon vector within bacteria. The BAC cassette can include bacterially-expressed sequences that assist in the use of bacterial strains, e.g., selectable genes, such as genes conferring bacterial resistance to antibiotics or toxins (e.g., chloramphenicol, tetracycline, ampicillin, and zeocin). The BAC cassette can further include reporter genes under the control of a eukaryotic promoter, such as a constitutive mammalian promoter. Examples of reporter genes can include, but are not limited to, LacZ encoding beta-galactosidase or a fluorescent protein. Examples of a fluorescent protein can include, but are not limited to, GFP, YFP, RFP, and analogues thereof. In some embodiments, an RFP can comprise mCherry. In some embodiments, analogues of GFP, YFP, or RFP can comprise iRFP, EGFP, or the like. Examples of a constitutive mammalian promoter can include, but are not limited to, an SV40, RSV, CMV, ubiquitin C (UbC), CAG, or Beta-actin promoter. In some embodiments, the amplicon vector may comprise a Flag-tagged luciferase. In some embodiments, the Flag-tagged luciferase expression may be under the control of CAG promoter. In some embodiments, the reporter genes may be under the control of an HSV promoter. For example, the reporter genes may be under the control of an HSV Class I gene promoter or an HSV Class II gene promoter. In some embodiments, the amplicon vector may comprise a GFP. In some embodiments, the GFP expression may be under the control of an oriS+IE4/5 promoter.

The plasmid backbone in the amplicon vector may further comprise a yeast artificial chromosome (YAC) cassette. The inclusion of such YAC cassette can facilitate propagation and manipulation of the amplicon vector within yeast. The YAC cassette can include yeast-expressed and non-expressed sequences that assist in the use of yeast strains, e.g., selectable genes, such as genes conferring yeast resistance to nutrient deficient growth media (e.g., uracil-deficient media, lysine-deficient media, and leucine-deficient media) and sequences required for propagation (e.g., centromere and replication origin). In the instance where a YAC cassette is positioned adjacent to a BAC cassette, this can be referred to as a BAC/YAC cassette.

The BAC, YAC, or BAC/YAC cassette can be inserted into the amplicon vector in any suitable location. The BAC cassette can be flanked by sequences facilitating removal of the BAC, YAC, or BAC/YAC cassette, such as by site-specific recombinase recognition sites/consensus sequences (e.g., those recognized by enzymes such as cre, dre, flp, KD, B2, B3, R, etc.). The inclusion of such sites facilitates excision of the BAC cassette, if desired, since BAC, YAC, or BAC/YAC sequences have been shown to reduce virus growth in cultured cells, stimulate an innate response, and promote transgene silencing in infected cells. Excision of the BAC, YAC, or BAC/YAC cassette also can increase the capacity for the vector to incorporate one or more transgenes, since BAC, YAC, or BAC/YAC cassettes are on the order of about 11-15 kb. The amplicon vector can have one or more (e.g., one, two, three, four, or five) consensus recognition sequences for a recombinase enzyme (e.g., loxP), particularly one not native to the HSV genome. Removal of a BAC, YAC, or BAC/YAC cassette using a cell line that expresses an appropriate site-specific recombinase for excising the BAC, YAC, or BAC/YAC cassette leaves a single copy of the one or more consensus sequences for a recombinase enzyme within the amplicon genome. The bacterial sequences may also be eliminated by inducible integrase-mediated “looping-out” and selective degradation of the bacterial elements flanked by compatible att sites, similar to plasmid-derived minicircle production but requiring a BAC, YAC, or BAC/YAC-compatible minicircle E. coli strain.

Replication defective (or deficient) HSV vectors are similar to HSV amplicons in that both are designed to deliver and express transgene(s) in the absence of the normal HSV gene expression cascade, but differ in transgene payload capacity and viral gene content. Specifically, while HSV amplicons described herein may enable ˜150 kb of payload and carry no viral protein coding genes, replication-defective vectors may provide at best ˜40 kb of transgene capacity given that the remainder of the genome encodes viral genes. It is not clear that all viral genes will remain silent under conditions that favor HSV reactivation and, thus, the complete elimination of all viral genes afforded in HSV amplicon vectors may improve the safety profile of the vector. As such, in a preferred embodiment, the HSV vectors of the present technology is an HSV amplicon.

The HSV amplicons described herein may be present as isolated DNA, DNA within a cell, or packaged in a viral envelope (e.g., a HSV amplicon particle). Provided herein are complementing packaging cell lines engineered to express one or more essential replication and/or packaging genes for production of HSV amplicon particles. Packaging cell lines described herein allows more space for payload or cargo sequence in HSV amplicon vectors as HSV amplicon vectors need to provide HSV replication and/or packaging signals, but not any essential replication or packaging genes. Upon transfection of the amplicon vector into the packaging cell line, infectious amplicon particles comprising one or more (e.g., one, two, three, four, or ranges of any of these values) tandem copies of the amplicon DNA surrounded by the HSV capsid, tegument, and envelope proteins may be produced.

### Engineering Packaging Cell Lines and Recombinant Genome

In one aspect, provided herein is a packaging cell line comprising a recombinant genome, wherein the recombinant genome may comprise (a) a first nucleic acid sequence comprising one or more Herpes Simplex Virus (HSV) Class I genes; and/or (b) a second nucleic acid sequence comprising one or more HSV Class I and Class II genes. In another aspect, provided herein is a packaging cell line comprising a recombinant genome, wherein the recombinant genome may comprise (a) a first nucleic acid sequence comprising one or more Herpes Simplex Virus (HSV) Class I genes and Class II genes; and/or (b) a second nucleic acid sequence comprising one or more HSV Class I genes.

In some embodiments, a first nucleic acid sequence or a second nucleic acid sequence may comprise a sequence with at least 50% sequence identity to one or more HSV Class I genes. In some embodiments, a first nucleic acid sequence or a second nucleic acid sequence may comprise a sequence with at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 98.5%, at least 98.6%, at least 98.7%, at least 98.8%, at least 98.9%, at least 99%, at least 99.1%, at least 99.2%, at least 99.3%, at least 99.4%, at least 99.5%, at least 99.6%, at least 99.7%, at least 99.8%, at least 99.8%, at least 99.9%, or 100% sequence identity to one or more HSV Class I genes. In some embodiments, the one or more HSV Class I genes may comprise ICP4, ICP27, ICP0, ICP22, ICP34.5, VP16, or a combination thereof. In some embodiments, the one or more HSV Class I genes may comprise ICP34.5, VP16, or a combination thereof. In some embodiments, the one or more HSV Class I genes may comprise ICP0, ICP34.5, VP16, or a combination thereof. In some embodiments, the one or more HSV Class I genes may comprise ICP4, ICP27, or a combination thereof. In some embodiments, the one or more HSV Class I genes may comprise ICP4, ICP0, ICP27, or a combination thereof.

Additionally, or alternatively, in some embodiments, a first nucleic acid sequence or a second nucleic acid sequence may comprise a sequence with at least 50% sequence identity to one or more HSV Class II genes. In some embodiments, a first nucleic acid sequence or a second nucleic acid sequence may comprise a sequence with at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 98.5%, at least 98.6%, at least 98.7%, at least 98.8%, at least 98.9%, at least 99%, at least 99.1%, at least 99.2%, at least 99.3%, at least 99.4%, at least 99.5%, at least 99.6%, at least 99.7%, at least 99.8%, at least 99.8%, at least 99.9%, or 100% sequence identity to one or more HSV Class II genes. In some embodiments, the one or more HSV Class II genes may comprise UL1, UL2, UL3, UL4, UL5, UL6, UL7, UL8, UL9, UL10, UL 11, UL 12, UL13, UL14, UL15, UL16, UL17, UL18, UL19, UL20, UL21, UL22, UL23, UL24, UL25, UL26, UL26.5, UL27, UL28, UL29, UL30, UL31, UL32, UL33, UL34, UL35, UL36, UL37, UL38, UL39, UL40, UL41, UL42, UL43, UL44, UL45, UL46, UL47, UL49, UL49A, UL50, UL51, UL52, UL53, UL55, UL56, US1, US2, US3, US4, USS, US6, US7, US8, US9, US10, US11. US12, or a combination thereof. In some embodiments, HSV Class II genes may comprise UL19, UL20, UL21, or a combination thereof. In some embodiments, HSV Class II genes ma comprise UL19, UL20, UL21, UL41, or a combination thereof.

In some embodiments, the one or more HSV Class I or HSV Class II genes in the first nucleic acid sequence or the second nucleic acid sequence may be separated by a cleavage signal. Non-limiting examples of a cleavage signal can include a 2A cleavage signal, including but not limited to P2A, E2A, F2A, or T2A. 2A cleavage signals and sequences thereof are well known in the art and any known sequence can be used. In some embodiments, the one or more HSV Class I or HSV Class II genes in the first nucleic acid sequence or the second nucleic acid sequence may be codon-optimized.

In some embodiments, the first nucleic acid sequence may further comprise a first one or more regulatory elements that are functionally connected to one or more HSV Class I genes. In some embodiments, the first one or more regulatory elements may comprise an enhancer, a promoter, a terminator, or a combination thereof.

In some embodiments, the first one or more regulatory elements may comprise a promoter. The promoter may be any promoter desired to control/regulate the expression of the one or more HSV Class I genes. In one embodiment, the promoter may comprise a natural or native promoter of the one or more HSV Class I genes comprising ICP4, ICP27, ICP0, ICP22, ICP34.5, VP16, or a combination thereof. In some embodiments, the promoter may comprise a natural or native promoter of the one or more HSV Class I genes comprising ICP34.5, VP16, or a combination thereof. In some embodiments, the promoter may comprise a natural or native promoter of the one or more HSV Class I genes comprising ICP0, ICP34.5, VP16, or a combination thereof. In some embodiments, the promoter may comprise a natural or native promoter of the one or more HSV Class I genes comprising ICP4, ICP27, or a combination thereof. In some embodiments, the promoter may comprise a natural or native promoter of the one or more HSV Class I genes comprising ICP4, ICP0, ICP27, or a combination thereof.

Other exemplary promoters may include, but are not limited to, a cell-specific or tissue-specific promoter (e.g., EOS, OCT4, Nanog, SOX2 (for neural stem cells), aMHC, Brachyury, Tau, GFAP, NSE, Synapsin I (for neurons), Apo A-I, Albumin, ApoE (for liver), MCK, SMC a-Actin, Myosin heavy chain, Myosin light chain (for muscle, etc.)), such as a promoter that specifically or preferentially expresses genes in a defined cell type (e.g., within a liver cell, lung cell, epithelial cell, cardiac cell, neural cell, skeletal muscle cell, embryonic, induced pluripotent, or other stem cell, cancer cell, etc.). In some embodiments, the promoters for use in sensory neurons may include, but are not limited to, TRPV1, CGRP, or NF200.

In some embodiments of the present disclosure, the promoter may comprise a constitutive mammalian promoter. Exemplary constitutive mammalian promoters may include, but are not limited to, SV40, CMV, CAG, EF1a, UbC, RSV, β-actin, or PGK.

In other embodiments, the promoter may comprise an inducible promoter. Inducible promoters are well-known in the art. Exemplary inducible promoters may include, but are not limited to, a tetracycline-inducible promoter or a doxycycline-inducible promoter. In some embodiments, the inducible promoter may be a Tet-On promoter. In some embodiments, a Tet-On promoter may include, but is not limited to, TRE3G or TRE-tight. In some embodiments, Tet-On promoter may comprise TRE3G. In some embodiments, Tet-On promoter may comprise TRE-tight. When a Tet-On promoter is used, expression of its cognate rtTA is needed. For example, use of TRE3G as the promoter necessitates expression of the cognate dox-sensitive transactivator protein, rtTA3G. The cognate rtTA can be expressed from a vector or from target cells or tissue (e.g., by introduction of a viral (e.g., AAV) vector encoding the rtTA into the target cells or tissue). In some embodiments, an inducible promoter can be used to control and/or manage the expression of a gene that may be toxic to host cells or packaging cell lines (e.g., VP16 and ICP0).

In some embodiments, the second nucleic acid sequence may further comprise a second one or more regulatory elements that are functionally connected to one or more HSV Class II genes. In some embodiments, the second one or more regulatory elements may comprise an enhancer, a promoter, a terminator, or a combination of thereof. In some embodiments, the second one or more regulatory elements may comprise the promoter. In some embodiments, the promoter may be any one of the cell-specific or tissue-specific promoters, constitutive mammalian promoters, and inducible promoters as discussed above. In some embodiments, the promoter may comprise a natural or native promoter of the one or more HSV Class II genes comprising UL1, UL2, UL3, UL4, UL5, UL6, UL7, UL8, UL9, UL 10, UL 11, UL 12, UL13, UL14, UL15, UL16, UL17, UL18, UL19, UL20, UL21, UL22, UL23, UL24, UL25, UL26, UL26.5, UL27, UL28, UL29, UL30, UL31, UL32, UL33, UL34, UL35, UL36, UL37, UL38, UL39, UL40, UL41, UL42, UL43, UL44, UL45, UL46, UL47, UL49, UL49A, UL50, UL51, UL52, UL53, UL55, UL56, US1, US2, US3, US4, US5, US6, US7, US8, US9, US10, US11, US12, or a combination thereof. In some embodiments, the promoter may comprise a natural or native promoter of the one or more HSV Class II genes comprising UL19, UL20, UL21, or a combination thereof. In some embodiments, the promoter may comprise a natural or native promoter of the one or more HSV Class II genes comprising UL19, UL20, UL21, UL41, or a combination thereof.

In some embodiments, the first nucleic acid sequence or the second nucleic acid sequence may not comprise a nucleic acid sequence comprising an HSV origin of replication (ori), an HSV packaging signal (pac), or a combination thereof. In some embodiments, the recombinant genome may not comprise a nucleic acid sequence comprising oriL/S. In some embodiments, the recombinant genome may not comprise a nucleic acid sequence comprising an HSV origin of replication (ori), an HSV packaging signal (pac), or a combination thereof. In some embodiments, the recombinant genome may not comprise a nucleic acid sequence comprising oriL/S.

The packaging cell line may comprise any suitable cell line. For example, the packaging cell line may comprise a mammalian cell line. In some embodiments, the packaging cell line may include, but is not limited to, a Human Bone Osteosarcoma Epithelial (U2OS) cell line, a Baby Hamster Kidney (BHK) cell line, a Human Embryonic Kidney (HEK 293) cell line, Henrietta Lacks (HeLa) cell line, Madin-Darby canine kidney (MDCK) cell line, Chinese hamster ovary (CHO) cell line, A549 lung carcinoma cell line, OVCAR3 human ovarian carcinoma cell line, C127 murine mammary tumor cell line, 3T3 mouse fibroblast cell line, U251 MG human glioblastoma cell line, SCC-4 human tongue squamous carcinoma cell line, nuclear-RFP transduced SK-OV-3 tumor cell line, Bowes human melanoma cell line, monkey COS-7 cell line, dhfr gene-deficient CHO cell line, mouse L cell line, mouse AtT-20 cell line, mouse myeloma cell line, rat GH3 cell line, human FL cell line, pluripotent stem cells such as iPS cell, ES cell and the like of human and other mammals, and primary cultured cells prepared from various tissues, or an African Green Monkey Kidney (Vero) cell line. In some embodiments, the packaging cell line may comprise a U2OS cell line. In some embodiments, the packaging cell line may comprise U2OS cells comprising ICP4, ICP27, ICP0, ICP22, ICP34.5, VP16, or a combination thereof. In some embodiments, the packaging cell line may comprise U2OS cells comprising ICP4 and ICP27 genes (U2OS-4/27). In some embodiments, the packaging cell line may comprise U2OS cells comprising ICP0, ICP4, and ICP27 genes (U2OS-0-4/27). In some embodiments, the packaging cell line may comprise U2OS cells comprising UL1, UL2, UL3, UL4, ULS. UL6, UL7, UL8, UL9, UL10, UL11, UL12, UL13, UL14, UL15, UL16, UL17, UL18, UL19, UL20, UL21, UL22, UL23, UL24, UL25, UL26, UL26.5, UL27, UL28, UL29, UL30, UL31, UL32, UL33, UL34, UL35, UL36, UL37, UL38, UL39, UL40, UL41, UL42, UL43, UL44, UL45, UL46, UL47, UL49, UL49A, UL50, UL51, UL52, UL53, UL55, UL56, US1, US2, US3, US4, US5, US6, US7, US8, US9, US10, US11, US12, or a combination thereof. In some embodiments, the packaging cell line may comprise U2OS cells comprising UL19, UL20, UL21, UL22, UL23, UL24, UL25, UL26, UL26.5, UL27, UL28, UL29, UL30, UL31, UL32, UL33, UL34, UL35, UL36, UL37, UL38, UL39, UL40, UL41, UL42, UL43, UL44, UL45, UL46, UL47, UL49, UL49A, UL50, UL51, UL52, UL53, UL55, UL56, US1, US2, US3, US4, US5, US6, US7, US8, US9, US10, US11, or a combination thereof. In some embodiments, the packaging cell line may comprise U2OS cells comprising UL19, UL20, UL21, UL22, UL23, UL24, UL25, UL26, UL26.5, UL27, UL28, UL29, UL30, UL31, UL32, UL33, UL34, UL35, UL36, UL37, UL38, UL39, UL40, UL41, UL42, UL43, UL44, UL45, UL46, UL47, UL49, UL49A, UL50, UL51, UL52, UL53, UL55, UL56, US1, US2, US3, US4, US5, US6, US7, US8, US9, US10, and US11. In some embodiments, the packaging cell line may comprise U2OS cells comprising UL19, UL20, UL21, or a combination thereof. In some embodiments, the packaging cell line may comprise U2OS cells comprising UL19, UL20, and UL21. In some embodiments, the packaging cell line may comprise U2OS cells comprising UL19, UL20, UL21, UL41, or a combination thereof. In some embodiments, the packaging cell line may comprise U2OS cells comprising UL19, UL20, UL21, and UL41.

In some aspects, nucleic acid sequence described herein may be introduced or inserted into a genomic safe harbor of the genome. As used herein, genomic safe harbors (GSH) refers to sites in the genome that are able to accommodate the integration of new genetic material in a manner that ensure that the newly inserted genetic elements: (i) function predictably, and/or (ii) do not cause alternations of the host genome that may pose a risk to the host cell organism. As such, genomic safe harbors may be used as sites for transgene insertion for functional genetics studies and therapeutic applications (e.g., gene therapy). Examples of genomic safe harbors in human include, but are not limited to, the adeno-associated virus site 1 (AAVS1), the chemokine (C—C motif) receptor 5 (CCR5) gene, a hypoxanthine phosphoribosyltransferase 1 (HPRT) locus, and the human ortholog of the mouse Rosa26 locus (or Rosa26 homolog locus). See Papapetrou and A. Schambach, Molecular Therapy 24 (4): 678-684 (2016). In some embodiments, the first nucleic acid sequence or the second nucleic acid sequence may be inserted in a first genomic safe harbor site (GSH1) of the recombinant genome. In some embodiments, the first genomic safe harbor site may comprise an AAVS1 locus, CCR5 locus, HPRT locus, or Rosa26 homolog locus. In some embodiments, the first genomic safe harbor site may comprise a Rosa26 homolog locus. In some embodiments, the first nucleic acid sequence or second nucleic acid sequence may be inserted in a second genomic safe harbor site (GSH2) of the recombinant genome. In some embodiments, the second genomic safe harbor may comprise an AAVS1 locus, CCR5 locus, HPRT locus, or Rosa26 homolog locus. In some embodiments, the second genomic safe harbor may comprise an AAVS1 locus.

In some embodiments, recombinase mediated cassette exchange (RMCE) may be used to integrate any of the nucleic acid sequence described herein into the genome of a packaging cell line to generate a recombinant genome. RMCE is a process in which site-specific recombinases exchange one gene cassette flanked by a pair of incompatible target sites for another cassette flanked by an identical pair of sites. Typically, one cassette is present in the host genome, whereas the other gene cassette is introduced into the host cell by chemical or biological means. RMCE uses recombinases (including, but not limited to, Cre or Flp recombinase) and 2 incompatible recombinase recognition sites (including, but not limited to, Cre or Flp recognition sites). For a non-limiting example, a Cre recombinase and the incompatible recognition sites loxP and lox2272 may be used.

A RMCE landing pad approach called Recombinase Writing of Iterative DNA and Trap Excision (REWRITE) can enable integrating large DNA sequence into host cells. Based on the REWRITE approach, a DNA construct may be integrated into a genome by contacting the genome with one or more agents configured to cleave the genome at a locus (e.g., genomic safe harbors), integrating a landing pad sequence into the genome at the locus, and integrating one or more cargo sequences (e.g., first nucleic acid sequence or second nucleic acid sequence) into the landing pad sequence, thereby integrating the DNA construct into the genome and generating a recombinant genome.

A landing pad sequence as described herein may comprise a recombinase recognition site, and may further comprise a gene (e.g., a gene encoding a recombinase and/or a gene encoding a selective marker such as a fluorescent protein), a regulatory element, or a combination thereof. In some embodiments, a landing pad sequence may comprise a nucleic acid sequence with at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 98.5%, at least 98.6%, at least 98.7%, at least 98.8%, at least 98.9%, at least 99%, at least 99.1%, at least 99.2%, at least 99.3%, at least 99.4%, at least 99.5%, at least 99.6%, at least 99.7%, at least 99.8%, at least 99.8%, at least 99.9%, or 100% sequence identity to SEQ ID NO: 4. In some embodiments, a landing pad sequence may comprise a sequence comprising SEQ ID NO: 4.

REWRITE has optimal features allowing for complete portability to any mammalian cells; any place in the genome including, but not limited to, silenced regions; allows for sequential integration of large DNA constructs towards the megabase scale; and includes a self-excising selection cassette for rapid and seamless removal of genetic scars, including but not limited to selectable markers. Site-specific nucleases (SSNs) or homology-directed recombination (HR) can be used to place REWRITE landing pad. SSN may comprise meganucleases, zinc-finger nucleases (ZFN), TAL effector nucleases (TALENs), or clustered regularly interspaced short palindromic repeats (CRISPR)/CRISPR-associated (Cas) system.

In another aspect, the present disclosure provides a method of producing a packaging cell line comprising introducing into a cell comprising a genome: (a) a first nucleic acid sequence comprising one or more HSV Class I genes of any embodiment disclosed above, and/or (b) a second nucleic acid sequence comprising one or more HSV Class I and Class II genes of any embodiment disclosed above. In another aspect, the present disclosure provides a method of producing a packaging cell line comprising introducing into a cell comprising a genome: (a) a first nucleic acid sequence comprising one or more HSV Class I genes and Class II genes of any embodiment disclosed above, and/or (b) a second nucleic acid sequence comprising one or more HSV Class I genes of any embodiment disclosed above.

Methods for introducing into a cell comprising a genome a heterologous gene to generate a recombinant genome are well-known to a person of ordinary skill in the art.

In some embodiments, the introducing may comprise (i) a recombination mediated cassette exchange; (ii) a homology directed repair; or (iii) a random integration.

In some embodiments, site-specific nucleases (SSNs) or homology-directed recombination (HR) can be used to insert any of nucleic acid sequence described herein into a recombinant genome. In some embodiments, HR can be used utilizing an endogenous homologous recombination machinery.

In some embodiments, SSNs may comprise meganucleases, zinc-finger nucleases (ZFN), TAL effector nucleases (TALENs), or clustered regularly interspaced short palindromic repeats (CRISPR)/CRISPR-associated (Cas) system. These four major classes of gene-editing techniques, namely, meganucleases, ZFNs, TALENs, CRISPR/Cas systems share a common mode of action in binding a user-defined sequence of DNA and mediating a double-stranded DNA break (DSB). DSB may then be repaired by HR, an event that introduces the homologous sequence from a donor DNA fragment, or by non-homologous end joining (NHEJ), when there is no donor DNA present.

In some embodiments, a CRISPR-Cas system may be used with a guide target sequence for genetic screening, targeted transcriptional regulation, targeted knock-in, and/or targeted genome editing, including base editing, epigenetic editing, and/or introducing double strand breaks (DSBs) for homologous recombination-mediated insertion of a nucleotide sequence. CRISPR-Cas system may comprise an endonuclease protein whose DNA-targeting specificity and cutting activity can be programmed by a short guide RNA or a duplex crRNA/TracrRNA. A CRISPR endonuclease may comprise a caspase effector nuclease, typically microbial Cas9 and a short guide RNA (gRNA) or an RNA duplex comprising a 18 to 20 nucleotide targeting sequence that directs the nuclease to a location of interest in the genome. Genome editing can refer to the targeted modification of a DNA sequence, including but not limited to, adding, removing, replacing, or modifying existing DNA sequences, and inducing chromosomal rearrangements or modifying transcription regulation elements (e.g., methylation/demethylation of a promoter sequence of a gene) to alter gene expression. As described above CRISPR-Cas system may require a guide system that can locate Cas protein to the target DNA site in the genome. In some instances, the guide system comprises a crispr RNA (crRNA) with a 17-20 nucleotide sequence that is complementary to a target DNA site and a trans-activating crRNA (tracrRNA) scaffold recognized by the Cas protein (e.g., Cas9). The 17-20 nucleotide sequence complementary to a target DNA site is referred to as a spacer while the 17-20 nucleotide target DNA sequence is referred to a protospacer. While crRNAs and tracrRNAs exist as two separate RNA molecules in nature, single guide RNA (sgRNA or gRNA) can be engineered to combine and fuse crRNA and tracrRNA elements into one single RNA molecule. Thus, in one embodiment, the gRNA may comprise two or more RNAs, e.g., crRNA and tracrRNA. In another embodiment, the gRNA may comprise a sgRNA comprising a spacer sequence for genomic targeting and a scaffold sequence for Cas protein binding. In some instances, the guide system may naturally comprise a sgRNA. For example, Cas12a/Cpf1 utilizes a guide system lacking tracrRNA and comprising only a crRNA containing a spacer sequence and a scaffold for Cas12a/Cpf1 binding. While the spacer sequence can be varied depending on a target site in the genome, the scaffold sequence for Cas protein binding can be identical for all gRNAs.

CRISPR-Cas systems described herein can comprise different CRISPR enzymes. For example, the CRISPR-Cas system can comprise Cas9, Cas12a/Cpf1, Cas12b/C2c1, Cas12c/C2c3, Cas12d/CasY, Cas12e/CasX, Cas12g, Cas12h, or Cas12i. In some non-limiting example embodiments, Cas enzymes include, but are not limited to, Cas1, Cas1B, Cas2, Cas3, Cas4, Cas5, Cas5d, Cas5t, Cas5h, Cas5a, Cas6, Cas7, Cas8, Cas8a, Cas8b, Cas8c, Cas9 (also known as Csn1 or Csx12), Cas10, Cas10d, Cas12a/Cpf1, Cas12b/C2c1, Cas12c/C2c3, Cas12d/CasY, Cas12e/CasX, Cas12f/Cas14/C2c10, Cas12g, Cas12h, Cas12i, Cas12k/C2c5, Cas13a/C2c2, Cas13b, Cas13c, Cas13d, C2c4, C2c8, C2c9, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csx11, Csf1, Csf2, CsO, Csf4, Csd1, Csd2, Cst1, Cst2, Csh1, Csh2, Csa1, Csa2, Csa3, Csa4, Csa5, GSU0054, Type II Cas effector proteins, Type V Cas effector proteins, Type VI Cas effector proteins. CARF, DinG, homologues thereof, or modified or engineered versions thereof such as dCas9 (endonuclease-dead Cas9) and nCas9 (Cas9 nickase that has inactive DNA cleavage domain). In some cases, the compositions, methods, devices, and systems, described herein, may use the Cas9 nuclease from Streptococcus pyogenes, of which amino acid sequences and structures are well known to those skilled in the art.

In some embodiments, (ii) the homology directed repair may comprise (a) a homology directed repair following a targeted double strand break generated by an endonuclease or (b) a homology directed repair with long homology arms and naturally occurring double strand breaks. In some embodiments, the endonuclease may comprise a Clustered regularly interspaced short palindromic repeats (CRISPR)-CRISPR-associated protein 9 (Cas9), Cas3, Cpf1, a zinc finger nuclease, or a transcription activator-like effector nuclease (TALEN). In some embodiments, the homology directed repair with long homology arms and naturally occurring double strand breaks may further comprise selection with a marker. In some embodiments, (iii) the random integration may further comprise a selection with a marker. In any of the above embodiments, the marker may comprise LacZ (encoding beta-galactosidase). CAT (encoding chloramphenicol acetyltransferase), PIGA (encoding phosphatidylinositol glycan anchor biosynthesis class A), HPRT1 (encoding hypoxanthine phosphoribosyltransferase 1), a bioluminescent oxidative enzyme-encoding gene (e.g., firefly luciferase, renilla luciferase, and analogs thereof such as AkaLumine-HCl, CycLuc1), a fluorescent protein-encoding gene (e.g., GFP, YFP, RFP such as mCherry, and analogues thereof such as iRFP, EGFP), or a resistance gene of blasticidin, neomycin/G418, hygromycin, puromycin, or zeocin. In some embodiments, the marker may be under the control of a eukaryotic promoter, such as a constitutive mammalian promoter. For example, the marker may be under the control of an SV40. RSV, CMV, ubiquitin C (UbC), CAG, or Beta-actin promoter. In some embodiments, the marker may be under the control of an HSV promoter. For example, the marker may be under the control of an HSV Class I gene promoter or an HSV Class II gene promoter. In some embodiments, the marker may comprise a Flag-tagged luciferase. In some embodiments, the Flag-tagged luciferase expression may be under the control of CAG promoter. In some embodiments, the marker may comprise a GFP. In some embodiments, the GFP expression may be under the control of an oriS+IE4/5 promoter. In some embodiments, the first nucleic acid sequence or the second nucleic acid sequence described herein can comprise one or more genes encoding a marker described above.

In some embodiments, the first nucleic acid sequence comprising one or more HSV Class I genes and/or regulatory elements described herein; and/or the second nucleic acid sequence comprising one or more HSV Class I and Class II genes and/or regulatory elements described herein may be comprised in a vector. In some embodiments, the first nucleic acid sequence comprising one or more HSV Class I genes and Class II genes and/or regulatory elements described herein; and/or the second nucleic acid sequence comprising one or more HSV Class I genes and/or regulatory elements described herein may be comprised in a vector. Examples of a vector may include, but are not limited to, a DNA vector, an RNA vector, a plasmid, a lentivirus vector, adenoviral vector, a Rous sarcoma viral (RSV) vector, an HSV vector, and a retrovirus vector. For a non-limiting example, the gene of interest (e.g., genes comprised in the first nucleic acid sequence and/or the second nucleic acid sequence as disclosed herein) with a selectable marker may be subcloned into a lentiviral vector. The host or packaging cell can be infected with the lentiviral vector, and selected for expression of the marker (e.g., blasticidin or zeocin resistance). Expression of the gene-of-interest (e.g., HSV Class I and/or Class I gene products) may be confirmed by any suitable methods or assays in the art (e.g., PCR or RNAseq). The packaging cell line comprising a recombinant genome of the present technology may be propagated and cloned to provide a clonal population.

In some aspects, the present disclosure provides a composition comprising the recombinant genome of any one of embodiments described herein. For example, the present disclosure provides a composition comprising a recombinant genome comprising a first nucleic acid sequence comprising one or more Herpes Simplex Virus (HSV) Class I genes described herein; and/or a second nucleic acid sequence comprising one or more HSV Class I and Class II genes described herein. For example, the present disclosure provides a composition comprising a recombinant genome comprising a first nucleic acid sequence comprising one or more Herpes Simplex Virus (HSV) Class I genes and Class II genes described herein; and/or a second nucleic acid sequence comprising one or more HSV Class I genes described herein. In some embodiments, the recombinant genome may not comprise a nucleic acid sequence comprising an HSV origin of replication (ori), an HSV packaging signal (pac). In some embodiments, the recombinant genome may not comprise a nucleic acid sequence comprising oriL/S.

In some embodiments, the first nucleic acid sequence or the second nucleic acid sequence comprising one or more HSV Class I genes or gene cassettes may comprise a homology arm sequence that is homologous to a sequence of a genomic safe harbor site, a Tet-on promoter sequence, a ICP0 gene, a ICP4 gene, and/or a ICP27 gene. In this embodiment, the expression of the ICP0 gene, the ICP4 gene, and/or the ICP27 gene may be under the control of Tet-On promoter. In some embodiments, the first nucleic acid sequence or the second nucleic acid sequence may comprise the ICP0 gene, the ICP4 gene, and/or the ICP27 gene separated by a 2A cleavage signal sequence. In some embodiments, the 2A cleavage signal sequence may comprise P2A, E2A, F2A, or T2A cleavage signal sequence. In some embodiments, the first nucleic acid sequence or the second nucleic acid sequence may comprise a sequence with at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 98.5%, at least 98.6%, at least 98.7%, at least 98.8%, at least 98.9%, at least 99%, at least 99.1%, at least 99.2%, at least 99.3%, at least 99.4%, at least 99.5%, at least 99.6%, at least 99.7%, at least 99.8%, at least 99.8%, at least 99.9%, or 100% sequence identity to a nucleic acid sequence of SEQ ID NO: 2. In some embodiments, the first nucleic acid sequence or the second nucleic acid sequence may comprise a sequence comprising SEQ ID NO: 2.

In some embodiments, the first nucleic acid sequence or the second nucleic acid sequence comprising one or more HSV Class I genes or gene cassettes may comprise a homology arm sequence that is homologous to a sequence of a genomic safe harbor site, a Tet-on promoter sequence, a ICP0 gene sequence, a ICP4 gene sequence, and/or a ICP27 gene sequence. In some embodiments, the first nucleic acid sequence or the second nucleic acid sequence may comprise the ICP0 gene, the ICP4 gene, and/or the ICP27 gene separated by a 2A cleavage signal sequence. In some embodiments, the 2A cleavage signal sequence may comprise P2A, E2A, F2A, or T2A cleavage signal sequence. In some embodiments, the ICP0 gene sequence, the ICP4 gene sequence, and/or the ICP27 gene sequence may further comprise a sequence encoding a tag downstream or at the 3′ end of the gene sequence. In some embodiments, ICP0, ICP4, and/or ICP27 protein may have a tag at the C-terminus, when translated. In some embodiments, the tag at the C-terminus can be used to sequester ICP0, ICP4, and/or ICP27 proteins in the cytoplasm until an agent is introduced. In some embodiments, the agent may comprise estrogen. In some embodiments, the first nucleic acid sequence or the second nucleic acid sequence may comprise a sequence with at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 98.5%, at least 98.6%, at least 98.7%, at least 98.8%, at least 98.9%, at least 99%, at least 99.1%, at least 99.2%, at least 99.3%, at least 99.4%, at least 99.5%, at least 99.6%, at least 99.7%, at least 99.8%, at least 99.8%, at least 99.9%, or 100% sequence identity to a nucleic acid sequence of SEQ ID NO: 3. In some embodiments, the first nucleic acid sequence or the second nucleic acid sequence may comprise a sequence comprising SEQ ID NO: 3.

In some embodiments, the first nucleic acid sequence or the second nucleic acid sequence comprising one or more HSV Class I genes or gene cassettes may comprise a homology arm sequence that is homologous to a sequence of genomic safe harbor site, a Tet-on promoter sequence, a ICP0 gene, a ICP4 gene, a ICP27 gene, and/or a VP16 gene. In this embodiment, the expression of the ICP0 gene, the ICP4 gene, the ICP27 gene, and/or VP16 gene may be under the control of Tet-On promoter. In some embodiments, the ICP0 gene, the ICP4 gene, and/or the ICP27 gene may be codon-optimized. In some embodiments, the first nucleic acid sequence or the second nucleic acid sequence may comprise the ICP0 gene, the ICP4 gene, and/or the ICP27 gene separated by a 2A cleavage signal sequence. In some embodiments, the 2A cleavage signal sequence may comprise P2A, E2A, F2A, or T2A cleavage signal sequence. In some embodiments, the first nucleic acid sequence or the second nucleic acid sequence comprising one or more HSV Class I genes may comprise a sequence with at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 98.5%, at least 98.6%, at least 98.7%, at least 98.8%, at least 98.9%, at least 99%, at least 99.1%, at least 99.2%, at least 99.3%, at least 99.4%, at least 99.5%, at least 99.6%, at least 99.7%, at least 99.8%, at least 99.8%, at least 99.9%, or 100% sequence identity to a nucleic acid sequence of SEQ ID NO: 7. In some embodiments, the first nucleic acid sequence or the second nucleic acid sequence may comprise a sequence comprising SEQ ID NO: 7.

In some embodiments, the first nucleic acid sequence or the second nucleic acid sequence comprising one or more HSV Class I genes or gene cassettes may comprise a homology arm sequence that is homologous to a sequence of genomic safe harbor site, a Tet-on promoter sequence, a ICP0 gene, a ICP4 gene, and/or a ICP27 gene. In this embodiment, the expression of the ICP0 gene, the ICP4 gene, and/or the ICP27 gene may be under the control of Tet-On promoter. In some embodiments, the ICP0 gene, the ICP4 gene, and/or the ICP27 gene may be codon-optimized. In some embodiments, the first nucleic acid sequence or the second nucleic acid sequence may comprise the ICP0 gene, the ICP4 gene, and/or the ICP27 gene separated by a 2A cleavage signal sequence. In some embodiments, the 2A cleavage signal sequence may comprise P2A, E2A, F2A, or T2A cleavage signal sequence. In some embodiments, the first nucleic acid sequence or the second nucleic acid sequence comprising one or more HSV Class I genes may comprise a sequence with at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 98.5%, at least 98.6%, at least 98.7%, at least 98.8%, at least 98.9%, at least 99%, at least 99.1%, at least 99.2%, at least 99.3%, at least 99.4%, at least 99.5%, at least 99.6%, at least 99.7%, at least 99.8%, at least 99.8%, at least 99.9%, or 100% sequence identity to a nucleic acid sequence of SEQ ID NO: 8. In some embodiments, the first nucleic acid sequence or the second nucleic acid sequence may comprise a sequence comprising SEQ ID NO: 8.

In some embodiments, the first nucleic acid sequence or the second nucleic acid sequence may comprise a homology arm sequence that is homologous to a sequence of genomic safe harbor site and a landing pad sequence. In some embodiments, the landing pad sequence may be designed to accept one or more Class II HSV genes or gene cassettes. In some embodiments, the first nucleic acid sequence or the second nucleic acid sequence may comprise a sequence with at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 98.5%, at least 98.6%, at least 98.7%, at least 98.8%, at least 98.9%, at least 99%, at least 99.1%, at least 99.2%, at least 99.3%, at least 99.4%, at least 99.5%, at least 99.6%, at least 99.7%, at least 99.8%, at least 99.8%, at least 99.9%, or 100% sequence identity to a nucleic acid sequence of SEQ ID NO: 4. In some embodiments, the first nucleic acid sequence or the second nucleic acid sequence may comprise a sequence comprising SEQ ID NO: 4.

In some embodiments, the first nucleic acid sequence or the second nucleic acid sequence comprising one or more HSV Class I genes and one or more HSV Class II genes may comprise one or more HSV Class II genes or gene cassettes. In some embodiments, the first nucleic acid sequence or the second nucleic acid sequence comprising one or more HSV Class II genes may comprise UL 1, UL2, UL3, UL4, UL5, UL6, UL7, UL8, UL9, UL 10, UL 11, UL 12, UL13, UL14, UL15, UL16, UL17, UL18, UL19, UL20, UL21, UL22, UL23, UL24, UL25, UL26, UL26.5, UL27, UL28, UL29, UL30, UL31, UL32, UL33, UL34, UL35, UL36, UL37, UL38, UL39, UL40, UL41, UL42, UL43, UL44, UL45, UL46, UL47, UL49, UL49A, UL50, UL51, UL52, UL53, UL55, UL56, US1, US2, US3, US4, US5, US6, US7, US8, US9, US10, US11, or US12. In some embodiments, the expression of the one or more HSV Class II genes may be under the control of native promoters of the HSV Class II genes. In some embodiments, the expression of the one or more HSV Class II genes may be under the control of non-native promoters of the HSV Class II genes. In some embodiments, the first nucleic acid sequence or the second nucleic acid sequence comprising one or more HSV Class I genes may comprise a ICP34.5 gene. In some embodiments, the expression of the ICP34.5 gene may be under the control of a CMV promoter. In some embodiments, the first nucleic acid sequence or the second nucleic acid sequence may not comprise on and/or pac sequences. In some embodiments, the first nucleic acid sequence or the second nucleic acid sequence may comprise a sequence with at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 98.5%, at least 98.6%, at least 98.7%, at least 98.8%, at least 98.9%, at least 99%, at least 99.1%, at least 99.2%, at least 99.3%, at least 99.4%, at least 99.5%, at least 99.6%, at least 99.7%, at least 99.8%, at least 99.8%, at least 99.9%, or 100% sequence identity to a nucleic acid sequence of SEQ ID NO: 5. In some embodiments, the first nucleic acid sequence or the second nucleic acid sequence may comprise a sequence comprising SEQ ID NO: 5.

In some embodiments, the first nucleic acid sequence or the second nucleic acid sequence comprising one or more HSV Class I genes and one or more HSV Class II genes may comprise a sequence with at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 98.5%, at least 98.6%, at least 98.7%, at least 98.8%, at least 98.9%, at least 99%, at least 99.1%, at least 99.2%, at least 99.3%, at least 99.4%, at least 99.5%, at least 99.6%, at least 99.7%, at least 99.8%, at least 99.8%, at least 99.9%, or 100% sequence identity to a nucleic acid sequence of SEQ ID NO: 6. In some embodiments, the first nucleic acid sequence or the second nucleic acid sequence may comprise a sequence comprising SEQ ID NO: 6.

In some embodiments, the first nucleic acid sequence or the second nucleic acid sequence comprising one or more HSV Class I genes and one or more HSV Class II genes may comprise one or more HSV Class II genes or gene cassettes. In some embodiments, the first nucleic acid sequence or the second nucleic acid sequence comprising one or more HSV Class II genes may comprise UL1, UL2, UL3, UL4, UL5, UL6, UL7, UL8, UL9, UL10, UL11, UL12, UL13, UL14, UL15, UL16, UL17, UL18, UL19, UL20, UL21, UL22, UL23, UL24, UL25, UL26, UL26.5, UL27, UL28, UL29, UL30, UL31, UL32, UL33, UL34, UL35, UL36, UL37, UL38, UL39, UL40, UL41, UL42, UL43, UL44, UL45, UL46, UL47, UL49, UL49A, UL50, UL51, UL52, UL53, UL55, UL56, US1, US2, US3, US4, US5, US6, US7, US8, US9, US10, US11, or US12. In some embodiments, the expression of the one or more HSV Class II genes may be under the control of native promoters of the HSV Class II genes. In some embodiments, the expression of the one or more HSV Class II genes may be under the control of non-native promoters of the HSV Class II genes. In some embodiments, the first nucleic acid sequence or the second nucleic acid sequence comprising one or more HSV Class I genes may comprise a ICP34.5 gene, a VP16 gene, or a combination thereof. In some embodiments, the expression of the ICP34.5 gene may be under the control of CMV promoter. In some embodiments, the expression of the VP16 gene may be under the control of Tet-On promoter. In some embodiments, the first nucleic acid sequence or the second nucleic acid sequence may not comprise ori and/or pac sequences. In some embodiments, the first nucleic acid sequence or the second nucleic acid sequence may comprise a sequence with at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 98.5%, at least 98.6%, at least 98.7%, at least 98.8%, at least 98.9%, at least 99%, at least 99.1%, at least 99.2%, at least 99.3%, at least 99.4%, at least 99.5%, at least 99.6%, at least 99.7%, at least 99.8%, at least 99.8%, at least 99.9%, or 100% sequence identity to a nucleic acid sequence of SEQ ID NO: 9. In some embodiments, the first nucleic acid sequence or the second nucleic acid sequence may comprise a sequence comprising SEQ ID NO: 9.

In some embodiments, the first nucleic acid sequence or the second nucleic acid sequence comprising one or more HSV Class I genes and one or more HSV Class II genes may comprise one or more HSV Class II genes or gene cassettes. In some embodiments, the first nucleic acid sequence or the second nucleic acid sequence comprising one or more HSV Class II genes may comprise UL1, UL2, UL3, UL4, UL5, UL6, UL7, UL8, UL9, UL10, UL11, UL12, UL13, UL14, UL15, UL16, UL17, UL18, UL19, UL20, UL21, UL22, UL23, UL24, UL25, UL26, UL26.5, UL27, UL28, UL29, UL30, UL31, UL32, UL33, UL34, UL35, UL36, UL37, UL38, UL39, UL40, UL41, UL42, UL43, UL44, UL45, UL46, UL47, UL49, UL49A, UL50, UL51, UL52, UL53, UL55, UL56, US1, US2, US3, US4, US5, US6, US7, US8, US9, US10, US11, or US12. In some embodiments, the expression of the one or more HSV Class II genes may be under the control of native promoters of the HSV Class II genes. In some embodiments, the expression of the one or more HSV Class II genes may be under the control of non-native promoters of the HSV Class II genes. In some embodiments, the first nucleic acid sequence or the second nucleic acid sequence comprising one or more HSV Class I genes may comprise a ICP34.5 gene. In some embodiments, the expression of the ICP34.5 gene may be under the control of CMV promoter. In some embodiments, the first nucleic acid sequence or the second nucleic acid sequence may not comprise on and/or pac sequences. In some embodiments, the first nucleic acid sequence or the second nucleic acid sequence may comprise a sequence with at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 98.5%, at least 98.6%, at least 98.7%, at least 98.8%, at least 98.9%, at least 99%, at least 99.1%, at least 99.2%, at least 99.3%, at least 99.4%, at least 99.5%, at least 99.6%, at least 99.7%, at least 99.8%, at least 99.8%, at least 99.9%, or 100% sequence identity to a nucleic acid sequence of SEQ ID NO: 10. In some embodiments, the first nucleic acid sequence or the second nucleic acid sequence may comprise a sequence comprising SEQ ID NO: 10.

In some embodiments, the first nucleic acid sequence or the second nucleic acid sequence comprising one or more HSV Class II genes may comprise one or more HSV Class II genes or gene cassettes. In some embodiments, the first nucleic acid sequence or the second nucleic acid sequence comprising one or more HSV Class II genes may comprise UL 1, UL2, UL3, UL4, UL5, UL6, UL7, UL8, UL9, UL10, UL11, UL12, UL13, UL14, UL15, UL16, UL17, UL18, UL19, UL20, UL21, UL22, UL23, UL24, UL25, UL26, UL26.5, UL27, UL28, UL29, UL30, UL31, UL32, UL33, UL34, UL35, UL36, UL37, UL38, UL39, UL40, UL41, UL42, UL43, UL44, UL45, UL46, UL47, UL49, UL49A, UL50, UL51, UL52, UL53, UL55, UL56, US1, US2, US3, US4, US5, US6, US7, US8, US9, US10, US11, or US12. In some embodiments, the expression of the one or more HSV Class II genes may be under the control of native promoters of the HSV Class II genes. In some embodiments, the expression of the one or more HSV Class II genes may be under the control of non-native promoters of the HSV Class II genes. In some embodiments, the first nucleic acid sequence or the second nucleic acid sequence may not comprise ori and/or pac sequences. In some embodiments, the first nucleic acid sequence or the second nucleic acid sequence may comprise a sequence with at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 98.5%, at least 98.6%, at least 98.7%, at least 98.8%, at least 98.9%, at least 99%, at least 99.1%, at least 99.2%, at least 99.3%, at least 99.4%, at least 99.5%, at least 99.6%, at least 99.7%, at least 99.8%, at least 99.8%, at least 99.9%, or 100% sequence identity to a nucleic acid sequence of SEQ ID NO: 17. In some embodiments, the first nucleic acid sequence or the second nucleic acid sequence may comprise a sequence comprising SEQ ID NO: 17.

In another aspect, provided herein is a cell or a cell line comprising the recombinant genome described herein. In some embodiments, the cell or the cell line may comprise a mammalian cell or a mammalian cell line. In some embodiments, the cell or the cell line may comprise a Human Bone Osteosarcoma Epithelial (U2OS) cell, a Baby Hamster Kidney (BHK) cell, a Human Embryonic Kidney (HEK 293) cell, Henrietta Lacks (HeLa) cell line, Madin-Darby canine kidney (MDCK) cell line, Chinese hamster ovary (CHO) cell line, A549 lung carcinoma cell line, OVCAR3 human ovarian carcinoma cell line, C127 murine mammary tumor cell line, 3T3 mouse fibroblast cell line, U251 MG human glioblastoma cell line, SCC-4 human tongue squamous carcinoma cell line, nuclear-RFP transduced SK-OV-3 tumor cell line, Bowes human melanoma cell line, monkey COS-7 cell line, dhfr gene-deficient CHO cell line, mouse L cell line, mouse AtT-20 cell line, mouse myeloma cell line, rat GH3 cell line, human FL cell line, pluripotent stem cells such as iPS cell, ES cell and the like of human and other mammals, and primary cultured cells prepared from various tissues, or an African Green Monkey Kidney (Vero) cell. In some embodiments, the cell or the cell line may comprise U2OS cells comprising ICP4, ICP27, ICP0, ICP22, ICP34.5. VP16, or a combination thereof. In some embodiments, the cell or the cell line may comprise U2OS cells comprising ICP4 and ICP27 genes (U2OS-4/27). In some embodiments, the cell or the cell line may comprise U2OS cells comprising ICP0, ICP4, and ICP27 genes (U2OS-0-4/27). In some embodiments, the cell or the cell line may comprise U2OS cells comprising UL1, UL2, UL3, UL4, UL5, UL6, UL7, UL8, UL9, UL10, UL11, UL12, UL13, UL14, UL15, UL16, UL17, UL18, UL19, UL20, UL21, UL22, UL23, UL24, UL25, UL26, UL26.5, UL27, UL28, UL29, UL30, UL31, UL32, UL33, UL34, UL35, UL36, UL37, UL38, UL39, UL40, UL41, UL42, UL43, UL44, UL45, UL46, UL47, UL49, UL49A, UL50, UL51, UL52, UL53, UL55, UL56, US1, US2, US3, US4, US5, US6, US7, US8, US9, US10, US11, US12, or a combination thereof. In some embodiments, the cell or the cell line may comprise U2OS cells comprising UL19, UL20, UL21, UL22, UL23, UL24, UL25, UL26, UL26.5, UL27, UL28, UL29, UL30, UL31, UL32, UL33, UL34, UL35, UL36, UL37, UL38, UL39, UL40, UL41, UL42, UL43, UL44, UL45, UL46, UL47, UL49, UL49A, UL50, UL51, UL52, UL53, UL55, UL56, US1, US2, US3, US4, US5, US6, US7, US8, US9, US10, US11, or a combination thereof. In some embodiments, the cell or the cell line may comprise U2OS cells comprising UL19, UL20, UL21, UL22, UL23, UL24, UL25, UL26, UL26.5, UL27, UL28, UL29, UL30, UL31, UL32, UL33, UL34, UL35, UL36, UL37, UL38, UL39, UL40, UL41, UL42, UL43, UL44, UL45, UL46, UL47, UL49, UL49A, UL50, UL51, UL52, UL53, UL55, UL56, US1, US2, US3, US4, US5, US6, US7, US8, US9, US10, and US11. In some embodiments, the cell or the cell line may comprise U2OS cells comprising UL19, UL20, UL21, or a combination thereof. In some embodiments, the cell or the cell line may comprise U2OS cells comprising UL19, UL20, and UL21. In some embodiments, the cell or the cell line may comprise U2OS cells comprising UL19, UL20, UL21, UL41, or a combination thereof. In some embodiments, the cell or the cell line may comprise U2OS cells comprising UL19, UL20, UL21, and UL41.

### Production of Herpes Simplex Virus (HSV) Amplicon Particles

In another aspect, the present disclosure provides a system for producing a HSV amplicon particle comprising (a) the packaging cell line of any one of the above embodiments; and (b) an amplicon vector of any one of the above embodiments.

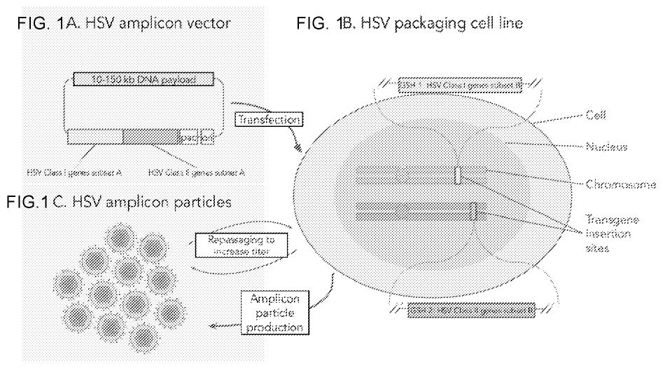

An example workflow of HSV amplicon particle production can be as follows (FIG. 1). An HSV amplicon vector is prepared. The HSV amplicon may comprise 10-150 kb of DNA payload (e.g., a transgene or a gene-of-interest, and/or regulatory elements such as an enhancer, a promoter, or a terminator, etc.), HSV origin of replication (ori), packaging (pac) sequences, one or more HSV class I genes, one or more HSV class II gene, and/or a marker. The HSV amplicon vector can be introduced into the HSV packaging cell line described herein by transfection using any method known to the skilled in the art or described herein. The HSV packaging cell line can have HSV Class I genes and/or HSV Class II genes inserted at 2 distinct genomic safe harbor (GSH) sites on different chromosomes. These are denoted as GSH 1 (yellow) and GSH 2 (orange) in FIG. 1. For example, HSV Class I genes can be inserted at GSH 1. For example, HSV Class II genes can be inserted at GSH 2. When the HSV amplicon is introduced into the HSV packaging cell line, HSV amplicon particles can be produced and isolated for applications.

In some embodiments, the amplicon vector of the present technology may comprise an amplicon plasmid comprising ori/pac sequences and a plasmid backbone, or minicircle DNA comprising ori/pac sequences. The on and pac sequences of the amplicon vector for use in the amplicon vector may be any suitable ori/pac sequences. The on/pac sequences may be placed into the amplicon vector in any suitable locations. In particular, the origin of replication may be any suitable origin of replication that allows for replication of the amplicon vector in the packaging cell line used for replication and packaging of the amplicon vector into the HSV amplicon particles. In some embodiments, origin of replication signals from HSV-1 or HSV-2 may be used. In some embodiments, the amplicon vector may comprise an HSV vector. For example, a replication deficient HSV vector may be used.

The pac sequence for use in the amplicon vector may be any suitable pac sequence, such that the amplicon vector can be packaged into a HSV amplicon particle that is capable of adsorbing to a cell or that can be transformed or transduced into a cell. Exemplary pac sequences may include, but are not limited to, pac sequences from HSV-1 and HSV-2.

In some embodiments, the amplicon vector may comprise one or more reporter genes. In some embodiments, the one or more reporter genes may be under the control of a eukaryotic promoter, such as a constitutive mammalian promoter. Examples of reporter genes can include, but are not limited to, LacZ encoding beta-galactosidase, CAT, HPRT1, PIGA, Luciferase, a fluorescent protein, blasticidin, neomycin/G418, hygromycin, puromycin, or zeocin. Examples of the fluorescent protein can include, but are not limited to, GFP, YFP, RFP, and analogues thereof. In some embodiments, an RFP can comprise mCherry. In some embodiments, analogues of GFP, YFP, or RFP can comprise iRFP, EGFP, or the like. Examples of a constitutive mammalian promoter can include, but are not limited to, an SV40, RSV. CMV, ubiquitin C (UbC), CAG, or Beta-actin promoter. In some embodiments, the one or more reporter genes may be under the control of an HSV promoter. For example, the reporter genes may be under the control of an HSV class I gene promoter or an HSV class II gene promoter. In some embodiments, the one or more reporter gene may be under the control of an oriS+IE4/5 promoter. In some embodiments, the amplicon vector may comprise a Flag-tagged luciferase. In some embodiments, the Flag-tagged luciferase expression may be under the control of CAG promoter. In some embodiments, the amplicon vector may comprise a GFP. In some embodiments, the GFP expression may be under the control of an oriS+IE4/5 promoter.

In some embodiments, the amplicon vector may comprise one or more HSV Class II genes. In some embodiments, the amplicon vector may comprise one or more HSV Class II genes comprising UL1, UL2, UL3, UL4, UL5, UL6, UL7, UL8, UL9, UL10, UL11, UL12, UL13, UL14, UL15, UL16, UL17, UL18, UL19, UL20, UL21, UL22, UL23, UL24, UL25, UL26, UL26.5, UL27, UL28, UL29, UL30, UL31, UL32, UL33, UL34, UL35, UL36, UL37, UL38, UL39, UL40, UL41, UL42, UL43, UL44, UL45, UL46, UL47, UL49, UL49A, UL50, UL51, UL52, UL53, UL55, UL56, US1, US2, US3, US4, US5, US6, US7, US8, US9, US10, US11, US12, or a combination thereof. In some embodiments, the amplicon vector may comprise UL1, UL2, UL3, UL4, UL5, UL6, UL7, UL8, UL9, UL10, UL 11, UL 12, UL13, UL14, UL15, UL16, UL 17, UL 18, UL19, or a combination thereof. In some embodiments, the amplicon vector may comprise one or more HSV Class II genes. In some embodiments, the amplicon vector may comprise UL20, UL21, UL22, UL23, UL24, UL25, UL26, UL26.5, UL27, UL28, UL29, UL30, UL31, UL32, UL33, UL34, UL35, UL36, UL37, UL38, UL39, UL40, UL41, UL42, UL43, UL44, UL45, UL46, UL47, UL49, UL49A, UL50, UL51, UL52, UL53, UL55, UL56, US1, US2, US3, US4, US5, US6, US7, US8, US9, US10, US11, US12, or a combination thereof. In some embodiments, the amplicon vector may comprise a mutation in or deletion of one or more HSV Class II genes. In one embodiment, an amplicon vector may comprise a mutation in or deletion of UL19, UL20, UL21, or a combination thereof. In another embodiment, an amplicon vector may comprise a mutation in or deletion of UL19, UL20, UL21, UL41, or a combination thereof. In some embodiments, HSV vectors may comprise a mutation in or deletion of UL19, UL20, and UL21. In some embodiments, HSV vectors may comprise a mutation in or deletion of UL19, UL20, UL21, and UL41.

In some embodiments, the amplicon vector may comprise one or more HSV Class I genes. In some embodiments, the amplicon vector may comprise ICP4, ICP27, ICP0, ICP22, ICP34.5, VP16, or a combination thereof. In one embodiment, an amplicon vector may comprise ICP4, ICP27. VP16, or a combination thereof. In another embodiment, an amplicon vector may comprise ICP4, ICP27, or a combination thereof. In yet another embodiment, an amplicon vector may comprise VP16. In some embodiments, an amplicon vector may comprise ICP4, ICP27, and VP16. In some embodiments, an amplicon vector may comprise ICP4, and ICP27.

In some embodiments, the amplicon vector comprises a sequence with at least 50% sequence identity to SEQ ID NO: 1, SEQ ID NO: 11, SEQ ID NO: 12, SEQ ID NO: 13, or SEQ ID NO: 14. In some embodiments, the amplicon vector comprises a sequence comprising SEQ ID NO: 1, SEQ ID NO: 11, SEQ ID NO: 12, SEQ ID NO: 13, or SEQ ID NO: 14.

In some embodiments, the amplicon vector may comprise a sequence with at least 50% sequence identity to a nucleic acid sequence of SEQ ID NO: 1. In some embodiments, the amplicon vector may comprise a sequence with at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 98.5%, at least 98.6%, at least 98.7%, at least 98.8%, at least 98.9%, at least 99%, at least 99.1%, at least 99.2%, at least 99.3%, at least 99.4%, at least 99.5%, at least 99.6%, at least 99.7%, at least 99.8%, at least 99.8%, at least 99.9%, or 100% sequence identity to a nucleic acid sequence of SEQ ID NO: 1. In some embodiments, the amplicon vector may comprise a sequence comprising SEQ ID NO: 1.

In some embodiments, the amplicon vector may comprise a sequence with at least 50% sequence identity to a nucleic acid sequence of SEQ ID NO: 11. In some embodiments, the amplicon vector may comprise a sequence with at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 98.5%, at least 98.6%, at least 98.7%, at least 98.8%, at least 98.9%, at least 99%, at least 99.1%, at least 99.2%, at least 99.3%, at least 99.4%, at least 99.5%, at least 99.6%, at least 99.7%, at least 99.8%, at least 99.8%, at least 99.9%, or 100% sequence identity to a nucleic acid sequence of SEQ ID NO: 11. In some embodiments, the amplicon vector may comprise a sequence comprising SEQ ID NO: 11.

In some embodiments, the amplicon vector may comprise a sequence with at least 50% sequence identity to a nucleic acid sequence of SEQ ID NO: 12. In some embodiments, the amplicon vector may comprise a sequence with at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 98.5%, at least 98.6%, at least 98.7%, at least 98.8%, at least 98.9%, at least 99%, at least 99.1%, at least 99.2%, at least 99.3%, at least 99.4%, at least 99.5%, at least 99.6%, at least 99.7%, at least 99.8%, at least 99.8%, at least 99.9%, or 100% sequence identity to a nucleic acid sequence of SEQ ID NO: 12. In some embodiments, the amplicon vector may comprise a sequence comprising SEQ ID NO: 12.

In some embodiments, the amplicon vector may comprise a sequence with at least 50% sequence identity to a nucleic acid sequence of SEQ ID NO: 13. In some embodiments, the amplicon vector may comprise a sequence with at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 98.5%, at least 98.6%, at least 98.7%, at least 98.8%, at least 98.9%, at least 99%, at least 99.1%, at least 99.2%, at least 99.3%, at least 99.4%, at least 99.5%, at least 99.6%, at least 99.7%, at least 99.8%, at least 99.8%, at least 99.9%, or 100% sequence identity to a nucleic acid sequence of SEQ ID NO: 13. In some embodiments, the amplicon vector may comprise a sequence comprising SEQ ID NO: 13.

In some embodiments, the amplicon vector may comprise a sequence with at least 50% sequence identity to a nucleic acid sequence of SEQ ID NO: 14. In some embodiments, the amplicon vector may comprise with a sequence at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 98.5%, at least 98.6%, at least 98.7%, at least 98.8%, at least 98.9%, at least 99%, at least 99.1%, at least 99.2%, at least 99.3%, at least 99.4%, at least 99.5%, at least 99.6%, at least 99.7%, at least 99.8%, at least 99.8%, at least 99.9%, or 100% sequence identity to a nucleic acid sequence of SEQ ID NO: 14. In some embodiments, the amplicon vector may comprise a sequence comprising SEQ ID NO: 14.

In any of the above embodiments, the amplicon vector may comprise at least one cargo nucleic acid sequence comprising a gene-of-interest or a transgene. In some embodiments, the at least one cargo nucleic acid sequence may comprise from about 10 kb to about 150 kb. For example, the at least one cargo nucleic acid sequence may comprise from about 10 kb to about 20 kb, from about 20 kb to about 30 kb, from about 30 kb to about 40 kb, from about 40 kb to about 50 kb, from about 50 kb to about 60 kb, from about 60 kb to about 70 kb, from about 70 kb to about 80 kb, from about 80 kb to about 90 kb, from about 90 kb to about 100 kb, from 100 about kb to about 110 kb, from about 110 kb to about 120 kb, from about 120 kb to about 130 kb, from about 130 kb to about 140 kb, or from about 140 kb to about 150 kb. In some embodiments, the at least one cargo nucleic acid sequence may comprise up to about 80 kb, up to about 85 kb, up to about 90 kb, up to, about 95 kb, up to about 100 kb, up to about 105 kb, up to about 110 kb, up to about 115 kb, up to about 120 kb, up to about 125 kb, up to about 130 kb, up to about 135 kb, up to about 140 kb, up to about 145 kb, or up to about 150 kb. In some embodiments, the at least one cargo nucleic acid sequence may comprise up to about 100 kb. In some embodiments, the at least one cargo nucleic acid sequence may comprise at least about 10 kb, at least about 15 kb, at least about 20 kb, at least about 25 kb, at least about 30 kb, at least about 35 kb, at least about 40 kb, at least about 45 kb, at least about 50 kb, at least about 55 kb, at least about 60 kb, at least about 65 kb, at least about 70 kb, at least about 75 kb, at least about 80 kb, at least about 85 kb, at least about 90 kb, at least about 95 kb, at least about 100 kb, at least about 105 kb, at least about 110 kb, at least about 115 kb, at least about 120 kb, at least about 125 kb, at least about 130 kb, at least about 135 kb, at least about 140 kb, at least about 145 kb, or at least about 150 kb. In some embodiments, the at least one cargo nucleic acid sequence may comprise about 45 kb.

Methods for inserting a cargo nucleic acid sequence into an amplicon vector is well-known to a person of ordinary skill in the art. See Annual Review of Biochemistry 89(1): 77-101 (2020). Those methods include, but are not limited to, restriction enzyme digestion cloning, Golden Gate Assembly, in vitro Cas9 digestion followed by Gibson assembly, Gateway cloning, or yeast recombination (or assembly in yeast). For non-limiting examples, the at least one cargo nucleic acid sequence may be inserted site-specifically in the amplicon vector by restriction enzyme digestion cloning, Golden Gate Assembly, in vitro Cas9 digestion followed by Gibson assembly, Gateway cloning, or in yeasto recombination assembly.

In some embodiments, the at least one cargo nucleic acid sequence may be inserted by restriction enzyme digestion cloning using one or more restriction enzyme sites comprising PacI, BbsI, BbsI-HF, BcoDI, BsaI-HF, BsmBI-v2, BsmI, BspQI, BsrDI, BsrI, BtsI-v2, BtsIMutI, EarI, Esp3I, SapI, AcuI, BaeI, BcgI, BpmI, BpuEI, BsaXI, BseRI, BsgI, BsmFI, BspCNI, BtgZI, CspCI, EcI, I, MmeI, NmeAIII, AlwI, BbvI, BccI, BceAI, BcI, VI, BfuAI, BmrI, BsmAI, BspMI, BtsCI, FauI, FokI, HgaI, HphI, HpyAV, MboII, MlyI, MnlI, PaqCI, PleI, SfaNI, I-CeuI, I-SceI, PI-PspI, PI-SceI, PmeI, or a combination thereof. In some embodiments, the at least one cargo nucleic acid sequences may be inserted site-specifically in the amplicon vector by restriction enzyme digestion cloning. Examples of restriction enzyme site sequences can include, but are not limited to, EcoRV, HindIII, and PstI. In some embodiments, the at least one cargo nucleic acid sequences may be inserted site-specifically in the amplicon vector by Golden Gate Cloning or Golden Gate assembly. In this embodiment, multiple DNA fragments are simultaneously and directionally assembled into a single piece using Type IIS restriction enzymes and T4 DNA ligase. Examples of Type IIS enzymes include, but are not limited to. BsaI, BsmBI, and BbsI.

Other non-restriction enzyme based DNA assembly methods such as Gibson assembly (following in vitro Cas9 digestion), Gateway cloning, and yeast recombination, may also be used to insert the at least one cargo nucleic acid sequences site-specifically in the amplicon vector. Those methods are well-known to a person of ordinary skill in the art.

In some embodiments, the at least one cargo nucleic acid sequence may be inserted by Gateway cloning using one or more Gateway cloning sites comprising attR1, attR2, or a combination thereof.

In some embodiments, the at least one cargo nucleic acid sequence may be inserted by in yeasto recombination assembly using one or more in yeasto recombination sites comprising NeoLVec, NeoRVec, or a combination thereof.

In some embodiments, the at least one cargo nucleic acid sequence may be inserted by in vitro Cas9 digestion using a Cas9 gRNA sequence followed by Gibson assembly.

In some embodiments, the at least one cargo nucleic acid sequence may comprise a transgene. In some embodiments, the transgene may comprise a gene editing system such CRISPR/Cas9 or nucleases, such as meganuclease, TALENS, zinc finger nuclease (ZFN), and Cpf1 DNA endonuclease. In some embodiments, the gene editing system may be used for the modification of epigenetic structures to silence genes or induce the expression of silent genes.

In some embodiments, the transgene may comprise CRISPR associated protein 9 (Cas9), Cas12a, Dystrophin (DYS), amylo-alpha-1, 6-glucosidase, 4-alpha-glucanotransferase (AGL), otoferlin (OTOF), myosin VIIA (MYO7A), ATP binding cassette subfamily A member 4 (ABCA4), coagulation factor VIII (F8), centrosomal protein 290 (CEP290), cadherin related 23 (CDH23), or centrosome, and basal body associated protein (ALMS1).

In some embodiments, the transgene may comprise a gene encoding one or more prophylactically- or therapeutically-active proteins, polypeptides, or other factors (e.g., non-coding RNA (ncRNA) such as siRNA or miRNA). In some embodiments, the transgene may comprise a gene encoding an agent that can enhance tumor killing activity (such as TRAIL or tumor necrosis factor (TNF)). In some embodiments, the transgene may comprise a gene encoding an agent that can be suitable for the treatment of conditions such as muscular dystrophy (a suitable transgene encodes Dystrophin), cardiovascular disease (suitable transgenes include, e.g., SERCA2a, GATA4, Tbx5, Mef2C, Hand2, Myocd, etc.), neurodegenerative disease (suitable transgenes include, e.g., NGF, BDNF, GDNF, NT-3. Huntingtin, etc.), chronic pain (suitable transgenes encode GlyRa1, an enkephalin, or a glutamate decarboxylase (e.g., GAD65, GAD67, or another isoform), lung disease (e.g., CFTR), or hemophilia (suitable transgenes encode. e.g., Factor VIII or Factor IX).

In some embodiments, the at least one cargo nucleic acid sequence may further comprise at least one regulatory element comprising an enhancer, a promoter, a terminator, or a combination thereof. In some embodiments, the at least one regulatory element may be a natural regulatory element of a transgene or a gene-of-interest encoded by the at least one cargo nucleic acid sequence. For example, the at least one cargo nucleic acid sequence may comprise a transgene or a gene-of-interest and its natural regulatory element sequence (e.g., an enhancer sequence, a promoter sequence, a terminator sequence, or a combination thereof). In some embodiments, the at least one cargo nucleic acid sequence may further comprise a sequence that is upstream or 5′ end of a transgene or a gene-of-interest in the genome (e.g., in its natural genomic context) and/or a sequence that is downstream or 3′ end of a transgene or a gene-of-interest in the genome (e.g., in its natural genomic context).

In some embodiments, the at least one cargo nucleic acid sequence may comprise a promoter. The promoter may be any promoter desired to control/regulate the expression of the at least one cargo nucleic acid sequence. In some embodiments, the promoter may comprise a natural promoter for a transgene or a gene-of-interest encoded by the at least one cargo nucleic acid sequence. In some embodiments, the at least one cargo nucleic acid sequence may further comprise a sequence that is upstream or 5′ end of a transgene or a gene-of-interest in the genome (e.g., in its natural genomic context). In some embodiments, the promoter may comprise a non-natural promoter. For example, the promoter may be from another gene for cell-specific or tissue-specific expression. For example, the promoter may be an inducible promoter for inducible expression. Exemplary promoters include but are not limited to a cell-specific or tissue-specific promoter (e.g., EOS, OCT4, Nanog, SOX2 (for neural stem cells), aMHC, Brachyury, Tau, GFAP, NSE, Synapsin I (for neurons), Apo A-I, Albumin, ApoE (for liver), MCK, SMC a-Actin, Myosin heavy chain, Myosin light chain (for muscle), etc.), such as a promoter that specifically or preferentially expresses genes in a defined cell type (e.g., within a liver cell, lung cell, epithelial cell, cardiac cell, neural cell, skeletal muscle cell, embryonic, induced pluripotent, or other stem cell, cancer cell, etc.). In some embodiments, the promoters for use in sensory neurons include btu are not limited to TRPV1, CGRP, and NF200.

In some embodiments of the present disclosure, the promoter of may be a constitutive mammalian promoter. Exemplary constitutive mammalian promoters include but are not limited to SV40, CMV, CAG, EF1a, UbC, RSV, β-actin, and PGK Depending on the activity of the promoter within the transgene, the transgene may be expressed in any type of mammalian cell that can be infected without cytotoxicity associated with viral gene expression.

In other embodiments, the promoter may be an inducible promoter. Inducible promoters are well-known in the art. Exemplary inducible promoters include but are not limited to a tetracycline-inducible promoter or a doxycycline-inducible promoter. In some embodiments, the inducible promoter is a Tet-On promoter. In some embodiments, a Tet-On promoter may include, but is not limited to, TRE3G or TRE-tight. When a Tet-On promoter is used, expression of its cognate rtTA is needed. For example, use of TRE3G as the promoter necessitates expression of the cognate dox-sensitive transactivator protein, rtTA3G. The cognate rtTA can be expressed from the amplicon, a separate vector, or from target cells or tissue (e.g., by introduction of a viral (e.g., AAV) vector encoding the rtTA into the target cells or tissue).

In some embodiments, at least one cargo nucleic acid sequence may comprise additional regulatory elements. For example, the cargo nucleic acid sequence may include one or more sites for binding of microRNA. In some embodiments, the cargo nucleic acid sequence may comprise tandem binding sites for the microRNAs, such as 2, 3, 4, 5, or 6 tandem sites. The presence of such sites, particularly tandem binding sites for such microRNAs, facilitates down-regulation of the transgene expression in certain cell types. Thus, for example, a transgene for expression in a cancer or tumor cell (which may be toxic to many cell types) can comprise binding sites for microRNAs of “normal” (e.g., non-malignant) cells, so that the expression of the transgene is suppressed in non-malignant cells.

The cargo nucleic acid sequence may be monocistronic (e.g., encoding a single protein or polypeptide) or polycistronic (e.g., encoding multiple proteins or polypeptides). All or part of the transcribed portion of the cargo nucleic acid sequence may also encode non-translated RNA, such as siRNA or miRNA. Multiple separate monocistronic or polycistronic transgene units (preferably two separate transgene units but possibly more (e.g., three, four, five, or more separate units)), each with its own respective promoter, translated sequence(s) or non-translated RNA sequence(s), and other regulatory elements may be used. In one embodiment, the cargo nucleic acid sequence may comprise a genomic gene that includes the gene's respective promoter, as well as introns and other regulatory sequences.

In some other embodiments, the cargo nucleic acid sequence or the payload may include a mammalian origin of replication, which leads to amplicon persistence in dividing cells post-transduction. Any suitable mammalian on may be used, including, but not limited to, those described in Hamlin. Bioessays 14(10): 651-659 (1992).

In some other embodiments, the cargo nucleic acid sequence or the payload may include Epstein-Barr Virus OriP and EBNA-1 sequences, which leads to amplicon persistence in dividing mammalian cells as described in Shan, Journal of Gene Medicine 8(12): 1400-6 (2006).

In another aspect, the present disclosure provides a method of producing a Herpes Simplex Virus (HSV) amplicon particle comprising transfecting the packaging cell line of any one of the above embodiments with an amplicon vector of any one of the above embodiments. In some embodiments, the HSV amplicon particle may comprise an HSV-1 or an HSV-2 amplicon particle.

The amplicon vector can be introduced into the packaging cell line via conventional transformation or transfection techniques. As used herein, the terms “transformation” and “transfection” can refer to a variety of art-recognized techniques for introducing foreign nucleic acid into a host cell, including calcium phosphate or calcium chloride co-precipitation, DEAE-dextran-mediated transfection, lipofection, electroporation, biolistics or viral-based transfection. Other methods used to transform mammalian cells include the use of polybrene, protoplast fusion, and the like. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals. The amplicon vector containing the DNA segments of interest can be transferred into the host cell by well-known methods, depending on the type of cellular host. In some embodiments, the amplicon vector can be introduced into the packaging cell line via transformation. In some cases, the transformation can comprise heat-shock transformation. In some cases, the transformation can comprise electroporation. In some cases, the transformation can comprise cell-cell fusion. In some embodiments, the amplicon vector can be introduced into the packaging cell line via transfection. In some cases, the transfection can comprise a physical transfection. In some non-limiting example embodiments, physical transfection includes: electroporation, sonoporation, optical transfection, or hydrodynamic delivery. In some cases, the transfection can use a chemical transfection method. In some non-limiting example embodiments, a chemical transfection method can include: calcium phosphate, cationic polymers, lipofection, fugene, or dendrimers. In some embodiments, the gene can be integrated into the genome via transduction (e.g., foreign nucleic acid introduced into a cell by a virus or viral vector).

In some embodiments, the number of HSV amplicon particles may be amplified by passaging. The method can further comprise isolating the HSV amplicon particles from the packaging cell line to provide a stock of amplicon particles. For a non-limiting example, the amplicon particles may be manufactured in roller bottles containing cells transduced with the HSV vector. After cell lysis, the HSV amplicon particles can be purified on a continuous Nycodenze gradient, and aliquoted and stored until needed. Viral stocks vary considerably in titer, depending largely on viral genotype and the protocol and cell lines used to prepare them. A stock of the HSV amplicon particles of the present technology may have a viral titer of about 10⁶ pfui/ml, about 10⁷ pfu/ml, about 10⁸ pfu/ml, about 10⁹ pfu/ml about 10¹⁰ pfu/ml, about 10¹¹ pfu/ml, about 10¹² pfu/ml, or more. Genome copy (gc) numbers provide a cell line-independent measure of the number of virus particles, but it includes defective particles. Typically, gc values for wild-type HSV-1 are several to 20×, up to 100×, higher than the pfu values of the same stocks. For mutant viruses, especially defective viruses grown on complementing cells, the value can increase to 10,000× higher or even more. Gc and pfu values increase proportionally with the size of the stock.

In some aspects, the present disclosure provides an amplicon particle produced by the method described herein. In some embodiments, the amplicon particle may further comprise HSV capsid, tegument, and/or envelope proteins.

### Transgene Expression

HSV can infect a wide variety of mammalian cells and thus the vectors of the present technology have broad applicability. In one aspect, the present disclosure provides a method of expressing a transgene in a cell, the method comprising contacting the cell with an HSV amplicon particle described in any of the above embodiments, e.g., an HSV amplicon particle produced by the method of any of the above embodiments, such that the transgene is expressed within the cell. In some embodiments, the HSV amplicon particles of the present technology are exposed to the cell under conditions suitable for the HSV amplicon particle to infect the cell. Once the cell is infected, the transgene will be transcribed (expressed) within the cell, provided the promoter within the transgene is one which is active in the cell and that the transgene is not suppressed by another regulatory mechanism.

In some embodiments, the contacting may occur in vivo. For example, the contacting may occur within an organism. The cell HSV amplicon particle is contacted with can be any type of desired cell, such as exocrine secretory cells (e.g., glandular cells, such as salivary gland cells, mammary gland cells, sweat gland cells, digestive gland cells, etc.), hormone secreting gland cells (e.g., pituitary cells, thyroid cells, parathyroid cells, adrenal cells, etc.), ectoderm-derived cells (e.g., keratinizing epithelial cells (e.g., making up the skin and hair), wet stratified barrier epithelial cells (e.g., of the cornea, tongue, oral cavity, gastrointestinal tract, urethra, vagina, etc.), cells of the nervous system (e.g., peripheral and central neurons, glia, etc.), mesoderm-derived cells, cells of many internal organs (such as kidney, liver, pancreas, heart, lung) bone marrow cells, and cancerous cells either within tumors or otherwise. In some embodiments, the cell is a liver cell, a lung cell, an epithelial cell, a neural cell, a cardiac cell, a muscle cell, a stem cell, or a cancer cell.

In some aspects, the method described herein can be used to treat a disease or a condition in a subject, wherein a transgene within the HSV or amplicon vector encodes one or more agents comprising prophylactically- or therapeutically-active proteins, polypeptides, or other factors (e.g., non-coding RNA (ncRNA) such as siRNA or miRNA). Thus, the present technology provides a method of treating a disease or a condition in a subject comprising administering the HSV amplicon particle to the subject in an amount that is sufficient to infect cells of the subject and therapeutically effective to treat the disease or the condition, such that the transgene encoding one or more agents comprising prophylactically or therapeutically active proteins, polypeptides, or other factors is expressed within the cells of the subject, thereby treating the disease or the condition. For example, the disease or condition may be a type of cancer, in which the transgene can encode an agent that enhances tumor killing activity (such as TRAIL or tumor necrosis factor (TNF)). As additional non-limiting example, the transgene may encode an agent suitable for the treatment of conditions such as muscular dystrophy (a suitable transgene encodes Dystrophin), cardiovascular disease (suitable transgenes include, e.g., SERCA2a, GATA4, Tbx5, Mef2C, Hand2, Myocd, etc.), neurodegenerative disease (suitable transgenes include, e.g., NGF, BDNF, GDNF, NT-3, Huntingtin, etc.), chronic pain (suitable transgenes encode GlyRa1, an enkephalin, or a glutamate decarboxylase (e.g., GAD65, GAD67, or another isoform), lung disease (e.g., CFTR), or hemophilia (suitable transgenes encode, e.g., Factor VIII or Factor IX).

In some other embodiments, the contacting may occur in vitro. Any type of cells can be infected in vitro, such as stem cells and fibroblasts (e.g., human dermal fibroblasts (HDF) or human lung fibroblasts (HLF)). Other non-limiting types of cells for use in vitro include keratinocytes, peripheral blood mononuclear cells, hematopoietic stem cells (CD34+), or mesenchymal stem/progenitor cells. In some embodiments, the transgene encodes one or more factors related to the differentiation of the cell. For example, expression of one or more of Oct4, Klf4, Sox2, c-Myc, L-Myc, dominant-negative p53, Nanog, Glis1, Lin28, TFIID, mir-302/367, or other miRNAs can cause the cell to become an induced pluripotent stem (iPS) cell. Alternatively, the transgene(s) can encode a factor for transdifferentiating the cells (e.g., one or more of GATA4, Tbx5. Mef2C, Myocd, Hand2, SRF, Mesp1, SMARCD3 (for cardiomyocytes); Ascl1, Nurr1, Lmx1A, Bm2, Mytl1, NeuroD1, FoxA2 (for neural cells), Hnf4a, Foxa1, Foxa2, or Foxa3 (for hepatic cells).

In any of the above embodiment, the cell may be a human cell.

### Pharmaceutical Compositions, Systems, Kits, and Therapeutic Applications

In some aspects, the present disclosure provides a pharmaceutical composition comprising any compositions described herein (e.g., any HSV amplicon particles described herein), and a pharmaceutically acceptable excipient.

A pharmaceutical composition can denote a mixture or solution comprising a therapeutically effective amount of an active pharmaceutical ingredient together with one or more pharmaceutically acceptable excipients to be administered to a subject in need thereof.

The term “pharmaceutically acceptable” can denote an attribute of a material which is useful in preparing a pharmaceutical composition that is generally safe, non-toxic, and neither biologically nor otherwise undesirable and is acceptable for veterinary as well as human pharmaceutical use. The term “pharmaceutically acceptable” can refer to a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained. A “pharmaceutically acceptable excipient” can denote any pharmaceutically acceptable ingredient in a pharmaceutical composition having no therapeutic activity and being non-toxic to the subject administered, such as disintegrators, binders, fillers, solvents, buffers, tonicity agents, stabilizers, antioxidants, surfactants, carriers, diluents, excipients, preservatives or lubricants used in formulating pharmaceutical products. Pharmaceutical compositions can facilitate administration of a compound to an organism and can be formulated in a conventional manner using one or more pharmaceutically acceptable inactive ingredients that can facilitate processing of active compounds or compositions into preparations that can be used pharmaceutically. A proper formulation is dependent upon the route of administration chosen and a summary of pharmaceutical compositions can be found, for example, in Remington: The Science and Practice of Pharmacy, Nineteenth Ed (Easton, Pa.: Mack Publishing Company, 1995); Hoover. John E., Remington's Pharmaceutical Sciences, Mack Publishing Co., Easton, Pennsylvania 1975; Liberman, H. A, and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference. In some embodiments, pharmaceutical compositions can be formulated by dissolving active substances (e.g., HSV amplicon particles described herein) in aqueous solution for injection into a subject in need thereof. In some embodiments, pharmaceutical compositions can be formulated by dissolving active substances (e.g., HSV amplicon particles described herein) in aqueous solution for direct injection into disease tissues or disease cells.

Provided herein are HSV amplicon particles, compositions, systems, and/or methods useful in the treatment of a disease or condition. In some aspects, HSV amplicon particles, compositions or systems are present or administered in an amount sufficient to treat or prevent a disease or condition. In some aspects, provided herein, is a method of treating a disease or condition comprising administering to a subject in need thereof HSV amplicon particles or pharmaceutical compositions described herein. In some aspects, provided herein, are HSV amplicon particles, compositions, systems, or pharmaceutical compositions described herein for use in a method of treating a disease or a condition in a subject in need thereof. In certain aspects, HSV amplicon particles, compositions, systems, kits, or pharmaceutical compositions described herein can be used in a gene therapy. In some aspects, provided herein, is the use of HSV amplicon particles, compositions, systems, or pharmaceutical compositions described herein for the manufacture of a medicament for treating a disease or a condition in a subject in need thereof.

Also provided herein are methods of treating a disease or condition in a subject in need thereof, comprising administering to the subject a therapeutically effective amount of any compositions or pharmaceutical compositions described herein. The terms “effective amount” or “therapeutically effective amount,” as used herein, can refer to a sufficient amount of an agent or a compound being administered which will relieve to some extent one or more of the symptoms of the disease or the condition being treated; for example a reduction and/or alleviation of one or more signs, symptoms, or causes of a disease, or any other desired alteration of a biological system. For example, an “effective amount” for therapeutic uses can be an amount of an agent that provides a clinically significant decrease in one or more disease symptoms. An appropriate “effective” amount may be determined using techniques, such as a dose escalation study, in individual cases.

The terms “treat,” “treating” or “treatment,” as used herein, can include alleviating, abating or ameliorating at least one symptom of a disease or a condition, preventing additional symptoms, inhibiting the disease or the condition. e.g., arresting the development of the disease or the condition, relieving the disease or the condition, causing regression of the disease or the condition, relieving a condition caused by the disease or the condition, or stopping the symptoms of the disease or the condition either prophylactically and/or therapeutically. In some embodiments, treating a disease or condition can comprise reducing the size of disease tissues or disease cells. In some embodiments, treating a disease or a condition in a subject can comprise increasing the survival of a subject. In some embodiments, treating a disease or condition can comprise reducing or ameliorating the severity of a disease, delaying onset of a disease, inhibiting the progression of a disease, reducing hospitalization of or hospitalization length for a subject, improving the quality of life of a subject, reducing the number of symptoms associated with a disease, reducing or ameliorating the severity of a symptom associated with a disease, reducing the duration of a symptom associated with a disease, preventing the recurrence of a symptom associated with a disease, inhibiting the development or onset of a symptom of a disease, or inhibiting of the progression of a symptom associated with a disease. In some embodiments, treating a cancer can comprise reducing the size of tumor or increasing survival of a patient with a cancer. In some embodiments, treating a cancer can comprise enhancing tumor killing activity. In some embodiments. HSV amplicon particles, compositions, pharmaceutical compositions, or systems described herein, may comprise a transgene encoding an agent that enhances tumor killing activity.

In some cases, a subject can encompass mammals. Examples of mammals can include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In some cases, the mammal may be a human. In some cases, the subject may be an animal. In some cases, an animal may comprise human beings and non-human animals. In one embodiment, a non-human animal may be a mammal, for example a rodent such as rat or a mouse. In another embodiment, a non-human animal may be a mouse. In some instances, the subject may be a mammal. In some instances, the subject may be a human. In some instances, the subject may be an adult, a child, or an infant. In some instances, the subject is may be a companion animal. In some instances, the subject may be a feline, a canine, or a rodent. In some instances, the subject may be a dog or a cat.

HSV amplicon particles or pharmaceutical compositions described herein may be administered as a combination therapy. Combination therapies with two or more therapeutic agents or therapies may use agents and therapies that work by different mechanisms of action. Combination therapies using agents or therapies with different mechanisms of action can result in additive or synergetic effects. Combination therapies may allow for a lower dose of each agent than is used in monotherapy, thereby reducing toxic side effects and/or increasing the therapeutic index of the agent(s). Combination therapies can decrease the likelihood that resistant disease cells will develop. In some instances, combination therapies comprise a therapeutic agent or therapy that affects the immune response (e.g., enhances or activates the response) and a therapeutic agent that affects (e.g., inhibits or kills) the disease cells. In some instances, combination therapies may comprise (i) recombinant RNA compositions or pharmaceutical compositions described herein; and (ii) one or more additional therapies known in the art for the diseases described herein. In some embodiments, HSV amplicon particles or pharmaceutical compositions described herein may be administered to a subject with a disease or condition prior to, concurrently with, and/or subsequently to, administration of one or more additional therapies for combination therapies. In some embodiments, the one or more additional therapies may comprise 1, 2, 3, or more additional therapeutic agents or therapies.

In some embodiments, a disease or condition may comprise a cancer or a non-cancer disease or a condition. For example, a disease or condition may comprise, but is not limited to, a cancer, muscular dystrophy, non-syndromic deafness, Stargardt Disease, Leber congenital amaurosis 10, Alstrom syndrome, a glycogen storage disease, Usher Syndrome 1D, Usher Syndrome 1B, a cardiovascular disease, a neurodegenerative disease, chronic pain, a lung disease, or hemophilia.

In one embodiment, a disease or condition may comprise a disease or condition caused by a genetic mutation. In some embodiments, a genetic mutation may comprise an insertion, a deletion, and/or a substitution of at least one nucleotide. In some embodiments, a genetic mutation may comprise an insertion, a deletion, and/or a substitution of at least one nucleotide in a gene encoding a protein. In some embodiments, an insertion, a deletion, and/or a substitution of at least one nucleotide in a gene encoding a protein may result in an insertion, a deletion, and/or a substitution of at least one amino acid in the protein encoded by the gene. In some embodiments, a genetic mutation may comprise an insertion, a deletion, and/or a substitution of at least one nucleotide in other elements important for gene expression. In some embodiments, a genetic mutation may be in a non-coding element important for gene expression. In some embodiments, a genetic mutation may be in a promoter sequence, an enhancer sequence, an insulator sequence, a terminator sequence, an operon sequence, a silencer sequence, a cis-acting element sequence, a trans-acting element sequence, or a sequence affecting structural configuration. In some embodiments, a disease or condition may be caused by a mutation in a gene, including missense, splicing, frameshift and nonsense mutations, as well as whole gene deletions, which result in deficient protein production. As a non-limiting example, Duchenne muscular dystrophy (DMD) is an X-linked recessive disorder that affects 1 in 3,500 males and is caused by mutations in the dystrophin gene.

In another embodiment, the described HSV amplicon particles, pharmaceutical compositions, or methods may be used to treat subjects having a disease or condition not caused by genetic mutation. In some embodiments, HSV amplicon particles, pharmaceutical compositions, or methods described herein may be used to treat subjects having a disease or condition, who can benefit from increased production of a protein encoded by a transgene comprised in HSV amplicon particles or pharmaceutical compositions described herein.

In some embodiments, the disease or condition may comprise a cancer. Examples of a cancer can include, but are not limited to, breast cancer, lung cancer, liver cancer, glioblastoma, melanoma, head and neck squamous cell carcinoma, renal cell carcinoma, neuroblastoma, Wilms tumor, retinoblastoma, rhabdomyosarcoma, osteosarcoma, Ewing sarcoma, bladder cancer, cervical cancer, colon cancer, rectal cancer, endometrial cancer, kidney cancer, mesothelioma, non-small cell lung cancer, nonmelanoma skin cancer, ovarian cancer, pancreatic cancer, prostate cancer, small cell lung cancer, colorectal cancer, thyroid cancer, sarcomas, carcinomas, lymphomas, laryngeal cancer, hypopharyngeal cancer, tonsil cancer, nasal cavity cancer, paranasal sinus cancer, nasopharyngeal cancer, metastatic squamous neck cancer with occult primary, lip cancer, oral cancer, oropharyngeal cancer, salivary gland cancer, brain tumors, esophageal cancer, eye cancer, parathyroid cancer, or leukemia, acute myeloid leukemia, colon cancer, gastric cancer, macular degeneration, acute monocytic leukemia, breast cancer, hepatocellular carcinoma, cone-rod dystrophy, alveolar soft part sarcoma, myeloma, skin melanoma, prostatitis, pancreatitis, pancreatic cancer, retinitis, adenocarcinoma, adenoiditis, adenoid cystic carcinoma, cataract, retinal degeneration, gastrointestinal stromal tumor, Wegener's granulomatosis, sarcoma, myopathy, prostate adenocarcinoma, Hodgkin's lymphoma, ovarian cancer, non-Hodgkin's lymphoma, multiple myeloma, chronic myeloid leukemia, acute lymphoblastic leukemia, renal cell carcinoma, transitional cell carcinoma, colorectal cancer, chronic lymphocytic leukemia, anaplastic large cell lymphoma, kidney cancer, breast cancer, cervical cancer, adenocarcinoma, melanoma (e.g., metastatic melanoma), liver cancer (e.g., hepatocellular carcinoma, hepatoblastoma, liver carcinoma), prostate cancer (e.g., prostate adenocarcinoma, androgen-independent prostate cancer, androgen-dependent prostate cancer, prostate carcinoma), sarcoma (e.g., leiomyosarcoma, rhabdomyosarcoma), brain cancer (e.g., glioma, a malignant glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma, anaplastic astrocytoma, juvenile pilocytic astrocytoma, a mixture of oligodendroglioma and astrocytoma elements), breast cancer (e.g., triple negative breast cancer, metastatic breast cancer, breast carcinoma, breast sarcoma, adenocarcinoma, lobular (small cell) carcinoma, intraductal carcinoma, medullary breast cancer, mucinous breast cancer, tubular breast cancer, papillary breast cancer, inflammatory breast cancer). Paget's disease, juvenile Paget's disease, lung cancer (e.g., KRAS-mutated non-small cell lung cancer, non-small cell lung cancer, squamous cell carcinoma (epidermoid carcinoma), adenocarcinoma, large-cell carcinoma, small cell lung cancer, lung carcinoma), pancreatic cancer (e.g., insulinoma, gastrinoma, glucagonoma, vipoma, somatostatin-secreting tumor, carcinoid tumor, islet cell tumor, pancreas carcinoma), skin cancer (e.g., skin melanoma, basal cell carcinoma, squamous cell carcinoma, melanoma, superficial spreading melanoma, nodular melanoma, lentigo malignant melanoma, acrallentiginous melanoma, skin carcinoma), cervical cancer (e.g., squamous cell carcinoma, adenocarcinoma, cervical carcinoma), ovarian cancer (e.g., ovarian epithelial carcinoma, borderline tumor, germ cell tumor, stromal tumor, ovarian carcinoma), cancer of the mouth, cancer of the nervous system (e.g., cancer of the central nervous system, a CNS germ cell tumor), goblet cell metaplasia, kidney cancer (e.g., renal cell cancer, adenocarcinoma, hypemephroma, Wilms' tumor, fibrosarcoma, transitional cell cancer (renal pelvis and/or uterer), renal cell carcinoma, renal carcinoma), bladder cancer (e.g., transitional cell carcinoma, squamous cell cancer, carcinosarcoma), stomach cancer (e.g., fungating (polypoid), ulcerating, superficial spreading, diffusely spreading, liposarcoma, fibrosarcoma, carcinosarcoma), uterine cancer (e.g., endometrial cancer, endometrial carcinoma, uterine sarcoma), cancer of the esophagus (e.g., squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma, esophageal carcinomas), colon cancer (e.g., colon carcinoma), cancer of the rectum (e.g., rectal cancers), colorectal cancer (e.g., colorectal carcinoma, metastatic colorectal cancer, hereditary nonpolyposis colorectal cancer, KRAS mutated colorectal cancer), gallbladder cancer (e.g., adenocarcinoma, cholangiocarcinoma, papillary cholangiocarcinoma, nodular cholangiocarcinoma, diffuse cholangiocarcinoma), testicular cancer (e.g., germinal tumor, seminoma, anaplastic testicular cancer, classic (typical) testicular cancer, spermatocytic testicular cancer, nonseminoma testicular cancer), embryonal carcinoma (e.g., teratoma carcinoma, choriocarcinoma (yolk-sac tumor)), gastric cancer (e.g., gastrointestinal stromal tumor, cancer of other gastrointestinal tract organs, gastric carcinomas), bone cancer (e.g., connective tissue sarcoma, bone sarcoma, cholesteatoma-induced bone osteosarcoma, Paget's disease of bone, osteosarcoma, chondrosarcoma, Ewing's sarcoma, malignant giant cell tumor, fibrosarcoma of bone, chordoma, periosteal sarcoma, soft-tissue sarcoma, angiosarcoma (hemangiosarcoma), fibrosarcoma, Kaposi's sarcoma, leiomyosarcoma, alveolar soft part sarcoma), liposarcoma, lymphangiosarcoma, neurilemmoma, rhabdomyosarcoma, synovial sarcoma, cancer of the lymph node (e.g., lymphangioendotheliosarcoma), adenoid cystic carcinoma, vaginal cancer (e.g., squamous cell carcinoma, adenocarcinoma, melanoma), vulvar cancer (e.g., squamous cell carcinoma, melanoma, adenocarcinoma, sarcoma, Paget's disease), cancer of other reproductive organs, thyroid cancer (e.g., papillary thyroid cancer, follicular thyroid cancer, medullary thyroid cancer, anaplastic thyroid cancer, thyroid carcinoma), salivary gland cancer (e.g., adenocarcinoma, mucoepidermoid carcinoma), eye cancer (e.g., ocular melanoma, iris melanoma, choroidal melanoma, ciliary body melanoma, retinoblastoma), penal cancers, oral cancer (e.g. squamous cell carcinoma, basal cancer), pharynx cancer (e.g., squamous cell cancer, verrucous pharynx cancer), cancer of the head, cancer of the neck, cancer of the throat, cancer of the chest, cancer of the spleen, cancer of skeletal muscle, cancer of subcutaneous tissue, adrenal cancer, pheochromocytoma, adrenocortical carcinoma, pituitary cancer, Cushing's disease, prolactin-secreting tumor, acromegaly, diabetes insipidus, myxosarcoma, osteogenic sarcoma, endotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma, papillary adenocarcinomas, ependymoma, optic nerve glioma, primitive neuroectodermal tumor, rhabdoid tumor, renal cancer, glioblastoma multiforme, neurofibroma, neurofibromatosis, pediatric cancer, neuroblastoma, malignant melanoma, carcinoma of the epidermis, polycythemia vera, Waldenstrom's macroglobulinemia, monoclonal gammopathy of undetermined significance, benign monoclonal gammopathy, heavy chain disease, pediatric solid tumor, Ewing's sarcoma, Wilms tumor, carcinoma of the epidermis, HIV-related Kaposi's sarcoma, rhabdomyosarcoma, thecomas, arrhenoblastomas, endometrial carcinoma, endometrial hyperplasia, endometriosis, fibrosarcomas, choriocarcinoma, nasopharyngeal carcinoma, laryngeal carcinoma, hepatoblastoma, Kaposi's sarcoma, hemangioma, cavernous hemangioma, hemangioblastoma, retinoblastoma, glioblastoma, Schwannoma, neuroblastoma, rhabdomyosarcoma, leiomyosarcoma, urinary tract carcinoma, abnormal vascular proliferation associated with phakomatoses, edema (such as that associated with brain tumors), Meigs' syndrome, pituitary adenoma, primitive neuroectodermal tumor, medulloblastoma, and acoustic neuroma. In some embodiments, a cancer may be a solid tumor or a solid cancer. In some embodiments, a solid tumor can be benign or malignant. In some embodiments, a cancer may be a liquid cancer.

HSV amplicon particles or pharmaceutical compositions described herein can be administered to a subject using any suitable methods known in the art. Suitable formulations for use and methods of delivery are generally well known in the art. For example, HSV amplicon particles or pharmaceutical compositions described herein can be administered to the subject in a variety of ways, including parenterally, intravenously, intradermally, intramuscularly, colonically, rectally, or intraperitoneally. In some embodiments, HSV amplicon particles or pharmaceutical compositions described herein can be administered by intraperitoneal injection, intramuscular injection, subcutaneous injection, or intravenous injection of the subject. In some embodiments, HSV amplicon particles or pharmaceutical compositions described herein can be administered parenterally, intravenously, intramuscularly or orally. In some embodiments, HSV amplicon particles or pharmaceutical compositions described herein can be administered via injection into a subject in need thereof. In some embodiments, HSV amplicon particles or pharmaceutical compositions described herein can be administered via injection into disease tissues or cells.

Any of HSV amplicon particles, compositions, systems, and pharmaceutical compositions described herein may be provided in a kit. The kit can contain, for example, materials and/or instruments for production of HSV amplicon particles and/or the storage and/or preservation of HSV amplicon particles, compositions, systems, and pharmaceutical compositions described herein (e.g., containers; materials for temperature control such as ice, ice packs, cold packs, dry ice, liquid nitrogen; chemical preservatives or buffers such as formaldehyde, formalin, paraformaldehyde, glutaraldehyde, alcohols such as ethanol or methanol, acetone, acetic acid, HOPE fixative (Hepes-glutamic acid buffer-mediated organic solvent protection effect), heparin, saline, phosphate buffered saline, TAPS, bicine, Tris, tricine, TAPSO, HEPES, TES, MOPS, PIPES, eadodylate, SSC, MES, phosphate buffer; protease inhibitors such as aprotinin, bestatin, calpain inhibitor I and II, chymostatin, E-64, leupeptin, alpha-2-macroglobulin, pefabloc SC, pepstatin, phenylmethanesufonyl fluoride, trypsin inhibitors; DNAse inhibitors such as 2-mercaptoethanol, 2-nitro-5-thicyanobenzoic acid, calcium, EGTA, EDTA, sodium dodecyl sulfate, iodoacetate, etc.; RNAse inhibitors such as ribonuclease inhibitor protein; double-distilled water; DEPC (diethyprocarbonate) treated water, etc.). The kit can be provided as, or contain, a suitable container for shipping. The shipping container can be an insulated container. The shipping container can be self-addressed to a collection agent (e.g., laboratory, medical center, genetic testing company, etc.). The kit can be provided to a subject for home use or use by a medical professional. Alternatively, the kit can be provided directly to a medical professional. The kit can contain instructions for use. The instruction manual may comprise instructions for the proper use of the kit.

In some instances, kits provided herein may be for preparing or producing HSV amplicon particles. In this embodiment, kits can comprise any packaging cell line described herein. In some embodiments, kits can comprise an amplicon vector. In some embodiments, an amplicon vector may comprise an origin of replication (ori) and/or a packaging signal (pac). In some embodiments, an amplicon vector may also comprise a cargo nucleic acid sequence described herein. In some embodiments, a cargo sequence may comprise a transgene. In some embodiments, a cargo nucleic acid sequence may comprise a regulatory element comprising an enhancer, a promoter, a terminator, or a thereof.

In some instances, kits provided herein may comprise HSV amplicon particles. In some embodiments, kits provided herein may comprise HSV amplicon particles for treatment of a disease or condition described herein. In some embodiments, HSV amplicon particles may comprise a cargo sequence may also comprise a cargo nucleic acid sequence described herein. In some embodiments, a cargo sequence may comprise a transgene. In some embodiments, a cargo nucleic acid sequence may comprise a regulatory element comprising an enhancer, a promoter, a terminator, or a combination thereof.

Any of HSV amplicon particles, compositions, systems, kits, and pharmaceutical compositions described herein may be provided together with an instruction manual. The instruction manual may comprise guidance for the skilled person how to use packaging cell lines described herein or how to produce HSV amplicon particles described herein in accordance with the disclosure. The instruction manual may comprise guidance for the skilled person or attending physician how to treat (or prevent) a disease or a disorder as described herein in accordance with the disclosure. In some embodiments, the instruction manual may comprise guidance as to the herein described mode of delivery/administration and delivery/administration regimen, respectively (e.g., route of delivery/administration, dosage regimen, time of delivery/administration, frequency of delivery/administration, etc.). In some embodiments, the instruction manual may comprise the instruction on how HSV amplicon particles or pharmaceutical compositions described herein can be administrated or injected and/or can be prepared for administration or injection. In principle, what has been described herein elsewhere with respect to the mode of delivery/administration and delivery/administration regimen, respectively, may be comprised as respective instructions in the instruction manual.

### OTHER EMBODIMENTS

In some aspects, provided herein is a packaging cell line comprising a recombinant genome, wherein the recombinant genome comprises a. a first nucleic acid sequence comprising one or more Herpes Simplex Virus (HSV) Class I genes; and b. a second nucleic acid sequence comprising one or more HSV Class I and Class II genes.

In some embodiments, the first nucleic acid sequence further comprises a first one or more regulatory elements. In some embodiments, the first nucleic acid sequence is in a first genomic safe harbor site of the recombinant genome. In some embodiments, the first genomic safe harbor site comprises a Rosa26 homolog locus. In some embodiments, the one or more HSV Class I genes comprise Infected-Cell Polypeptide 4 (ICP4), ICP27, ICP0, ICP34.5, VP16, or a combination thereof.

In some embodiments, the first one or more regulatory elements comprise an enhancer, a promoter, a terminator, or a combination thereof. In some embodiments, the first one or more regulatory elements comprise the promoter. In some embodiments, the promoter comprises an inducible promoter. In some embodiments, the inducible promoter comprises a tetracycline-inducible promoter or a doxycycline-inducible promoter. In some embodiments, the inducible promoter comprises Tet-On promoter comprising TRE3G or TRE-tight.

In some embodiments, the second nucleic acid sequence is in a second genomic safe harbor site of the recombinant genome. In some embodiments, the second genomic safe harbor comprises an adeno-associated virus site 1 (AAVS1) locus.

In some embodiments, the one or more HSV Class II genes comprise UL 1, UL2, UL3, UL4, UL5, UL6, UL7, UL8, UL9, UL10, UL11, UL12, UL13, UL14, UL15, UL16, UL17, UL18, UL19, UL20, UL21, UL22, UL23, UL24, UL25, UL26, UL26.5, UL27, UL28, UL29, UL30, UL31, UL32, UL33, UL34, UL35, UL36, UL37, UL38, UL39, UL40, UL41, UL42, UL43, UL44, UL45, UL46, UL47, UL49, UL49A, UL50, UL51, UL52, UL53, UL55, UL56, US1, US2, US3, US4, US5, US6, US7, US8, US9, US10, US11, US12, or a combination thereof.

In some embodiments, the second nucleic acid sequence further comprises a second one or more regulatory elements. In some embodiments, the second one or more regulatory elements comprise an enhancer, a promoter, a terminator, or a combination thereof. In some embodiments, the second one or more regulatory elements comprise the promoter. In some embodiments, the promoter comprises a natural promoter for HSV Class II genes comprising UL1, UL2, UL3, UL4, UL5, UL6, UL7, UL8, UL9, UL10, UL11, UL12, UL13, UL14, UL15, UL16, UL17, UL18, UL19, UL20, UL21, UL22, UL23, UL24, UL25, UL26, UL26.5, UL27, UL28, UL29, UL30, UL31, UL32, UL33, UL34, UL35, UL36, UL37, UL38, UL39, UL40, UL41, UL42, UL43, UL44, UL45, UL46, UL47, UL49, UL49A, UL50, UL51, UL52, UL53, UL55, UL56, US1, US2, US3, US4, US5, US6, US7, US8, US9, US10, US11, US12, or a combination thereof.

In some embodiments, the recombinant genome does not comprise a nucleic acid sequence comprising an HSV origin of replication (ori), an HSV packaging signal (pac), or a combination thereof. In some embodiments, the recombinant genome does not comprise a nucleic acid sequence comprising oriL/S. In some embodiments, the first nucleic acid sequence comprises a sequence with at least 50% sequence identity to SEQ ID NO: 2, SEQ ID NO: 3, SEQ ID NO: 7, or SEQ ID NO: 8. In some embodiments, the first nucleic acid sequence comprises a sequence comprising SEQ ID NO: 2, SEQ ID NO: 3, SEQ ID NO: 7, or SEQ ID NO: 8. In some embodiments, the second nucleic acid sequence comprises a sequence with at least 50% sequence identity to SEQ ID NO: 5, SEQ ID NO: 6, SEQ ID NO: 9, SEQ ID NO: 10, or SEQ ID NO: 17. In some embodiments, the second nucleic acid sequence comprises a sequence comprising SEQ ID NO: 5, SEQ ID NO: 6, SEQ ID NO: 9, SEQ ID NO: 10, or SEQ ID NO: 17.

In some embodiments, the packaging cell line comprises a mammalian cell line. In some embodiments, the packaging cell line is a Human Bone Osteosarcoma Epithelial (U2OS) cell line, a Baby Hamster Kidney (BHK) cell line, a Human Embryonic Kidney (HEK 293) cell line, or an African Green Monkey Kidney (Vero) cell line. In some embodiments, the U2OS cell line comprises U2OS cells comprising ICP4 and ICP27 genes (U2OS-4/27). In some embodiments, the U2OS cell line comprises U2OS cells comprising ICP0, ICP4, and ICP27 genes (U2OS-0-4/27).

In some aspects, provided herein is a composition comprising a recombinant genome, wherein the recombinant genome comprises a. a first nucleic acid sequence comprising one or more Herpes Simplex Virus (HSV) Class I genes; and b. a second nucleic acid sequence comprising one or more HSV Class I and Class II genes.

In some aspects, provided herein is a method of producing a packaging cell line comprising introducing into a cell comprising a genome: a. a first nucleic acid sequence comprising one or more Herpes Simplex Virus (HSV) Class I genes; and b. a second nucleic acid sequence comprising one or more HSV Class I and Class II genes.

In some aspects, provided herein is a system for producing a Herpes Simplex Virus (HSV) amplicon particle comprising: a. any packaging cell line described herein; and b. an amplicon vector. In some embodiments, the amplicon vector comprises at least one cargo nucleic acid sequence. In some embodiments, the at least one cargo nucleic acid sequence comprises from about 10 to about 150 kb. In some embodiments, the at least one cargo nucleic acid sequence comprises up to 100 kb.

In some embodiments, the at least one cargo nucleic acid sequence is inserted site-specifically in the amplicon vector by restriction enzyme digestion cloning, Golden Gate Assembly, in vitro Cas9 digestion followed by Gibson assembly, Gateway cloning, or in yeasto recombination assembly. In some embodiments, the at least one cargo nucleic acid sequence is inserted by restriction enzyme digestion cloning using one or more restriction enzyme sites comprising PacI, BbsI, BbsI-HF, BcoDI, BsaI-HF, BsmBI-v2, BsmI, BspQI, BsrDI, BsrI, BtsI-v2, BtsIMutI, EarI, Esp3I, SapI, AcuI, BaeI, BcgI, BpmI, BpuEI, BsaXI, BseRI, BsgI, BsmFI, BspCNI, BtgZI, CspCI, EcI, I, MmeI, NmeAIII, AwI, BbvI, BccI, BceAI, BcI, VI, BfuAI, BmrI, BsmAI, BspMI, BtsCI, FauI, FokI, HgaI, HphI, HpyAV, MboII, MlyI, MnlI, PaqCI, PleI, SfaNI, I-CeuI, I-SceI, PI-PspI, PI-SceI, PmeI, or a combination thereof. In some embodiments, the at least one cargo nucleic acid sequence is inserted by Gateway cloning using one or more Gateway cloning sites comprising attR1, attR2, or a combination thereof. In some embodiments, the at least one cargo nucleic acid sequence is inserted by in yeasto recombination assembly using one or more in yeasto recombination sites comprising NeoLVec, NeoRVec, or a combination thereof. In some embodiments, the at least one cargo nucleic acid sequence is inserted by in vitro Cas9 digestion followed by Gibson assembly using a Cas9 gRNA sequence.

In some embodiments, the at least one cargo nucleic acid sequence comprises a transgene. In some embodiments, the transgene comprises CRISPR associated protein 9 (Cas9), Cas3, Cas12a, Dystrophin (DYS), amylo-alpha-1, 6-glucosidase, 4-alpha-glucanotransferase (AGL), otoferlin (OTOF), myosin VIIA (MYO7A), ATP binding cassette subfamily A member 4 (ABCA4), coagulation factor VIII (F8), centrosomal protein 290 (CEP290), cadherin related 23 (CDH23), or centrosome and basal body associated protein (ALMS1). In some embodiments, the transgene comprises a gene encoding an agent for treating a cancer, muscular dystrophy, non-syndromic deafness, Stargardt Disease, Leber congenital amaurosis 10, Alstrom syndrome, a glycogen storage disease, Usher Syndrome 1D, Usher Syndrome 1B, a cardiovascular disease, a neurodegenerative disease, chronic pain, a lung disease, or hemophilia.

In some embodiments, the at least one cargo nucleic acid sequence further comprises at least one regulatory element comprising an enhancer, a promoter, a terminator, or a combination thereof. In some embodiments, the amplicon vector comprises an origin of replication (ori) and a packaging signal (pac). In some embodiments, the HSV amplicon particle comprises an HSV-1 or an HSV-2 amplicon particle.

In some embodiments, the amplicon vector comprises a sequence with at least 50% sequence identity to SEQ ID NO: 1, SEQ ID NO: 11, SEQ ID NO: 12, SEQ ID NO: 13, or SEQ ID NO: 14. In some embodiments, the amplicon vector comprises a sequence comprising SEQ ID NO: 1, SEQ ID NO: 11, SEQ ID NO: 12, SEQ ID NO: 13, or SEQ ID NO: 14. In some embodiments, the amplicon vector comprises a sequence with at least 50% sequence identity to SEQ ID NO: 1. In some embodiments, the amplicon vector comprises a sequence comprising SEQ ID NO: 1. In some embodiments, the amplicon vector comprises a sequence with at least 50% sequence identity to SEQ ID NO: 11. In some embodiments, the amplicon vector comprises a sequence comprising SEQ ID NO: 11. In some embodiments, the amplicon vector comprises a sequence with at least 50% sequence identity to SEQ ID NO: 12. In some embodiments, the amplicon vector comprises a sequence comprising SEQ ID NO: 12. In some embodiments, the amplicon vector comprises a sequence with at least 50% sequence identity to SEQ ID NO: 13. In some embodiments, the amplicon vector comprises a sequence comprising SEQ ID NO: 13. In some embodiments, the amplicon vector comprises a sequence with at least 50% sequence identity to SEQ ID NO: 14. In some embodiments, the amplicon vector comprises a sequence comprising SEQ ID NO: 14.

In some aspects, provided herein is a method of producing a Herpes Simplex Virus (HSV) amplicon particle comprising transfecting any packaging cell line described herein with an amplicon vector. In some embodiments, the method further comprises isolating the HSV amplicon particle from the packaging cell line.

In some embodiments, the HSV amplicon particle comprises an HSV-1 or an HSV-2 amplicon particle. In some embodiments, the amplicon vector comprises an origin of replication (ori) and a packaging signal (pac).

In some embodiments, the amplicon vector further comprises at least one cargo nucleic acid sequence. In some embodiments, the at least one cargo nucleic acid sequence comprises a transgene. In some embodiments, the transgene comprises CRISPR associated protein 9 (Cas9), Cas3, Cas12a, Dystrophin (DYS), amylo-alpha-1, 6-glucosidase, 4-alpha-glucanotransferase (AGL), otoferlin (OTOF), myosin VIIA (MYO7A), ATP binding cassette subfamily A member 4 (ABCA4), coagulation factor VIII (F8), centrosomal protein 290 (CEP290), cadherin related 23 (CDH23), or centrosome and basal body associated protein (ALMS1). In some embodiments, the transgene comprises a gene encoding an agent for treating a cancer, muscular dystrophy, non-syndromic deafness, Stargardt Disease, Leber congenital amaurosis 10, Alstrom syndrome, a glycogen storage disease, Usher Syndrome 1D, Usher Syndrome 1B, a cardiovascular disease, a neurodegenerative disease, chronic pain, a lung disease, or hemophilia.

In some embodiments, the at least one cargo nucleic acid sequence further comprises at least one regulatory element comprising an enhancer, a promoter, a terminator, or a combination thereof. In some embodiments, the amplicon vector comprises a sequence with at least 50% sequence identity to SEQ ID NO: 1, SEQ ID NO: 11, SEQ ID NO: 12, SEQ ID NO: 13, or SEQ ID NO: 14. In some embodiments, the amplicon vector comprises a sequence comprising SEQ ID NO: 1, SEQ ID NO: 11, SEQ ID NO: 12, SEQ ID NO: 13, or SEQ ID NO: 14. In some embodiments, the amplicon vector comprises a sequence with at least 50% sequence identity to SEQ ID NO: 1. In some embodiments, the amplicon vector comprises a sequence comprising SEQ ID NO: 1. In some embodiments, the amplicon vector comprises a sequence with at least 50% sequence identity to SEQ ID NO: 11. In some embodiments, the amplicon vector comprises a sequence comprising SEQ ID NO: 11. In some embodiments, the amplicon vector comprises a sequence with at least 50% sequence identity to SEQ ID NO: 12. In some embodiments, the amplicon vector comprises a sequence comprising SEQ ID NO: 12. In some embodiments, the amplicon vector comprises a sequence with at least 50% sequence identity to SEQ ID NO: 13. In some embodiments, the amplicon vector comprises a sequence comprising SEQ ID NO: 13. In some embodiments, the amplicon vector comprises a sequence with at least 50% sequence identity to SEQ ID NO: 14. In some embodiments, the amplicon vector comprises a sequence comprising SEQ ID NO: 14.

In some aspects, provided herein, is a Herpes Simplex Virus (HSV) amplicon particle produced by any method described herein. In some embodiments, the HSV further comprises HSV capsid, tegument, and envelope proteins.

In some aspects, provided herein is a pharmaceutical composition comprising any HSV amplicon particle described herein, and a pharmaceutically acceptable carrier.

In some aspects, provided herein is a cell comprising any HSV amplicon particle described herein.

In some aspects, provided herein is a kit comprising any HSV amplicon particle described herein or any packaging cell line described herein and an instruction manual for using the kit.

In some aspects, provided herein is a kit for preparing an HSV amplicon particle comprising (i) any packaging cell line described herein and (ii) an amplicon vector comprising an origin of replication (ori) and a packaging signal (pac); and an instruction manual for using the kit. In some embodiments, the amplicon vector further comprises at least one cargo sequence. In some embodiments, the at least one cargo sequence comprises a transgene selected from the group consisting of CRISPR associated protein 9 (Cas9), Cas3, Cas12a, Dystrophin (DYS), amylo-alpha-1, 6-glucosidase, 4-alpha-glucanotransferase (AGL), otoferlin (OTOF), myosin VIIA (MYO7A), ATP binding cassette subfamily A member 4 (ABCA4), coagulation factor VIII (F8), centrosomal protein 290 (CEP290), cadherin related 23 (CDH23), or centrosome and basal body as-sociated protein (ALMS1). In some embodiments, the at least one cargo sequence comprises a transgene encoding an agent for treating a cancer, muscular dystrophy, non-syndromic deafness. Stargardt Disease, Leber congenital amaurosis 10. Alstrom syndrome, a glycogen storage disease, Usher Syndrome 1D, Usher Syndrome 1B, a cardiovascular disease, a neurodegenerative disease, chronic pain, a lung disease, or hemophilia.

In some embodiments, the at least one cargo nucleic acid sequence further comprises at least one regulatory element comprising an enhancer, a promoter, a terminator, or a combination thereof. In some embodiments, the amplicon vector comprises a sequence with at least 50% sequence identity to SEQ ID NO: 1, SEQ ID NO: 11, SEQ ID NO: 12, SEQ ID NO: 13, or SEQ ID NO: 14. In some embodiments, the amplicon vector comprises a sequence comprising SEQ ID NO: 1, SEQ ID NO: 11, SEQ ID NO: 12, SEQ ID NO: 13, or SEQ ID NO: 14. In some embodiments, the amplicon vector comprises a sequence with at least 50% sequence identity to SEQ ID NO: 1. In some embodiments, the amplicon vector comprises a sequence comprising SEQ ID NO: 1. In some embodiments, the amplicon vector comprises a sequence with at least 50% sequence identity to SEQ ID NO: 11. In some embodiments, the amplicon vector comprises a sequence comprising SEQ ID NO: 11. In some embodiments, the amplicon vector comprises a sequence with at least 50% sequence identity to SEQ ID NO: 12. In some embodiments, the amplicon vector comprises a sequence comprising SEQ ID NO: 12. In some embodiments, the amplicon vector comprises a sequence with at least 50% sequence identity to SEQ ID NO: 13. In some embodiments, the amplicon vector comprises a sequence comprising SEQ ID NO: 13. In some embodiments, the amplicon vector comprises a sequence with at least 50% sequence identity to SEQ ID NO: 14. In some embodiments, the amplicon vector comprises a sequence comprising SEQ ID NO: 14.

In some aspects, provided herein, is a method of expressing a transgene in a cell, the method comprising contacting the cell with an HSV amplicon particle produced by any method described herein or any HSV amplicon particle described herein. In some embodiments, the contacting occurs in vivo. In some embodiments, the cell is a liver cell, a lung cell, an epithelial cell, a neural cell, a cardiac cell, a muscle cell, a stem cell, or a cancer cell. In some embodiments, the contacting occurs in vitro. In some embodiments, the cell is a human cell.

In some aspects, provided herein is a method of treating a disease or condition in a subject in need thereof, the method comprising administering to the subject therapeutically effective amount of a Herpes Simplex Virus (HSV) amplicon particle produced by any method described herein, any HSV amplicon particle described herein, or any pharmaceutical composition provided herein, wherein the therapeutically effective amount comprises an amount sufficient to infect cells of the subject such that at least one transgene is expressed in the subject.

In some embodiments, the disease or condition comprises a cancer, muscular dystrophy, non-syndromic deafness. Stargardt Disease, Leber congenital amaurosis 10, Alstrom syndrome, a glycogen storage disease, Usher Syndrome 1D, Usher Syndrome 1B, a cardiovascular disease, a neurodegenerative disease, chronic pain, a lung disease, or hemophilia. In some embodiments, the disease or condition comprises a disease or condition caused by a genetic mutation. In some embodiments, the disease or condition is a cancer. In some embodiments, the at least one transgene encodes an agent that enhances tumor killing activity. In some embodiments, the subject is a human.

In some aspects, provided herein is a use of any packaging cell line described herein or any system described herein for producing a Herpes Simplex Virus (HSV) amplicon particle. In some embodiments, the producing comprises transfecting the packaging cell line with an amplicon vector.

In some embodiments, the producing further comprises isolating the HSV amplicon particle from the packaging cell line. In some embodiments, the HSV amplicon particle comprises an HSV-1 or an HSV-2 amplicon particle. In some embodiments, the amplicon vector comprises an origin of replication (ori) and a packaging signal (pac).

In some embodiments, the amplicon vector further comprises at least one cargo nucleic acid sequence. In some embodiments, the at least one cargo nucleic acid sequence comprises a transgene. In some embodiments, the transgene comprises CRISPR associated protein 9 (Cas9), Cas3, Cas12a, Dystrophin (DYS), amylo-alpha-1, 6-glucosidase, 4-alpha-glucanotransferase (AGL), otoferlin (OTOF), myosin VIIA (MYO7A), ATP binding cassette subfamily A member 4 (ABCA4), coagulation factor VIII (F8), centrosomal protein 290 (CEP290), cadherin related 23 (CDH23), or centrosome and basal body associated protein (ALMS1). In some embodiments, the transgene comprises a gene encoding an agent for treating a cancer, muscular dystrophy, non-syndromic deafness, Stargardt Disease, Leber congenital amaurosis 10. Alstrom syndrome, a glycogen storage disease, Usher Syndrome 1D, Usher Syndrome 1B, a cardiovascular disease, a neurodegenerative disease, chronic pain, a lung disease, or hemophilia.

In some embodiments, the at least one cargo nucleic acid sequence further comprises at least one regulatory element comprising an enhancer, a promoter, a terminator, or a combination thereof. In some embodiments, the amplicon vector comprises a sequence with at least 50% sequence identity to SEQ ID NO: 1, SEQ ID NO: 11, SEQ ID NO: 12, SEQ ID NO: 13, or SEQ ID NO: 14. In some embodiments, the amplicon vector comprises a sequence comprising SEQ ID NO: 1, SEQ ID NO: 11, SEQ ID NO: 12, SEQ ID NO: 13, or SEQ ID NO: 14. In some embodiments, the amplicon vector comprises a sequence with at least 50% sequence identity to SEQ ID NO: 1. In some embodiments, the amplicon vector comprises a sequence comprising SEQ ID NO: 1. In some embodiments, the amplicon vector comprises a sequence with at least 50% sequence identity to SEQ ID NO: 11. In some embodiments, the amplicon vector comprises a sequence comprising SEQ ID NO: 11. In some embodiments, the amplicon vector comprises a sequence with at least 50% sequence identity to SEQ ID NO: 12. In some embodiments, the amplicon vector comprises a sequence comprising SEQ ID NO: 12. In some embodiments, the amplicon vector comprises a sequence with at least 50% sequence identity to SEQ ID NO: 13. In some embodiments, the amplicon vector comprises a sequence comprising SEQ ID NO: 13. In some embodiments, the amplicon vector comprises a sequence with at least 50% sequence identity to SEQ ID NO: 14. In some embodiments, the amplicon vector comprises a sequence comprising SEQ ID NO: 14.

In some aspects, provided herein is a use of a Herpes Simplex Virus (HSV) amplicon particle produced by any method described herein, any HSV amplicon particle described herein, or any pharmaceutical composition described herein for treating a disease or condition in a subject.

In some embodiments, the disease or condition comprises a cancer, muscular dystrophy, non-syndromic deafness, Stargardt Disease, Leber congenital amaurosis 10, Alstrom syndrome, a glycogen storage disease, Usher Syndrome 1D, Usher Syndrome 1B, a cardiovascular disease, a neuro-degenerative disease, chronic pain, a lung disease, or hemophilia. In some embodiments, the disease or condition comprises a disease or condition caused by a genetic mutation. In some embodiments, the disease or condition is a cancer. In some embodiments, the subject is a human.

In some aspects, provided herein is a composition comprising a recombinant genome, wherein the recombinant genome comprises: a. a first nucleic acid sequence comprising one or more Herpes Simplex Virus (HSV) Class I genes; and b. a second nucleic acid sequence comprising one or more HSV Class I and Class II genes.

In some embodiments, the first nucleic acid sequence further comprises a first one or more regulatory elements. In some embodiments, the first nucleic acid sequence is in a first genomic safe harbor site of the recombinant genome. In some embodiments, the first genomic safe harbor site comprises a Rosa26 homolog locus.

In some embodiments, the one or more HSV Class I genes comprise Infected-Cell Polypeptide 4 (ICP4), ICP27, ICP0, ICP34.5, VP16, or a combination thereof. In some embodiments, the first one or more regulatory elements comprise an enhancer, a promoter, a terminator, or a combination thereof. In some embodiments, the first one or more regulatory elements comprise the promoter. In some embodiments, the promoter comprises an inducible promoter. In some embodiments, the inducible promoter comprises a tetracycline-inducible promoter or a doxycycline-inducible promoter. In some embodiments, the inducible promoter comprises Tet-On promoter comprising TRE3G or TRE-tight.

In some embodiments, the second nucleic acid sequence is in a second genomic safe harbor site of the recombinant genome. In some embodiments, the second genomic safe harbor comprises an adeno-associated virus site 1 (AAVS1) locus.

In some embodiments, the one or more HSV Class II genes comprise UL 1, UL2, UL3, UL4, UL5, UL6, UL7, UL8, UL9, UL10, UL11, UL12, UL13, UL14, UL15, UL16, UL17, UL18, UL19, UL20, UL21, UL22, UL23, UL24, UL25, UL26, UL26.5, UL27, UL28, UL29, UL30, UL31, UL32, UL33, UL34, UL35, UL36, UL37, UL38, UL39, UL40, UL41, UL42, UL43, UL44, UL45, UL46, UL47, UL49, UL49A, UL50, UL51, UL52, UL53, UL55, UL56, US1, US2, US3, US4, US5, US6, US7, US8, US9, US10, US11, US12, or a combination thereof.

In some embodiments, the second nucleic acid sequence further comprises a second one or more regulatory elements. In some embodiments, the second one or more regulatory elements comprise an enhancer, a promoter, a terminator, or a combination thereof. In some embodiments, the second one or more regulatory elements comprise the promoter. In some embodiments, the promoter comprises a natural promoter for HSV Class II genes comprising UL1, UL2, UL3, UL4, UL5, UL6, UL7, UL8, UL9, UL10, UL11, UL12, UL13, UL14, UL15, UL16, UL17, UL18, UL19, UL20, UL21, UL22, UL23, UL24, UL25, UL26, UL26.5, UL27, UL28, UL29, UL30, UL31, UL32, UL33, UL34, UL35, UL36, UL37, UL38, UL39, UL40, UL41, UL42, UL43, UL44, UL45, UL46, UL47, UL49, UL49A, UL50, UL51, UL52, UL53, UL55, UL56, US1, US2, US3, US4, US5, US6, US7, US8, US9, US10, US11, US12, or a combination thereof.

In some embodiments, the recombinant genome does not comprise a nucleic acid sequence comprising an HSV origin of replication (ori), an HSV packaging signal (pac), or a combination thereof. In some embodiments, the recombinant genome does not comprise a nucleic acid sequence comprising oriL/S. In some embodiments, the first nucleic acid sequence comprises a sequence with at least 50% sequence identity to SEQ ID NO: 2, SEQ ID NO: 3. SEQ ID NO: 7, or SEQ ID NO: 8. In some embodiments, the first nucleic acid sequence comprises a sequence comprising SEQ ID NO: 2, SEQ ID NO: 3, SEQ ID NO: 7, or SEQ ID NO: 8. In some embodiments, the second nucleic acid sequence comprises a sequence with at least 50% sequence identity to SEQ ID NO: 5, SEQ ID NO: 6, SEQ ID NO: 9, SEQ ID NO: 10, or SEQ ID NO: 17. In some embodiments, the second nucleic acid sequence comprises a sequence comprising SEQ ID NO: 5, SEQ ID NO: 6, SEQ ID NO: 9, SEQ ID NO: 10, or SEQ ID NO: 17.

In some aspects, provided herein is a cell or a cell line comprising any composition described herein. In some embodiments, the cell or the cell line comprises a mammalian cell or a mammalian cell line. In some embodiments, the cell or the cell line comprises a Human Bone Osteo-sarcoma Epithelial (U2OS) cell, a Baby Hamster Kidney (BHK) cell, a Human Embryonic Kidney (HEK 293) cell, or an African Green Monkey Kidney (Vero) cell. In some embodiments, the cell or the cell line comprises an U2OS cell comprising ICP4 and ICP27 genes (U2OS-4/27). In some embodiments, the cell of the cell line comprises an U2OS cell comprising ICP0, ICP4, and ICP27 genes (U2OS-0-4/27).

In some aspect, provided herein is a kit comprising any composition described herein or any cell or cell line described herein and an instruction manual for using the kit. In some aspects, provided herein is a use of any composition described herein for producing a packaging cell line. In some aspects, provided herein is a method of producing a packaging cell line comprising introducing any composition described herein into a cell.

In some aspects, provided herein is a method of producing a packaging cell line comprising introducing into a cell comprising a genome: a. a first nucleic acid sequence comprising one or more Herpes Simplex Virus (HSV) Class I genes; and b. a second nucleic acid sequence comprising one or more HSV Class I and Class II genes. In some embodiments, the cell comprises a mammalian cell. In some embodiments, the mammalian cell comprises a Human Bone Osteosarcoma Epithelial (U2OS) cell, a Baby Hamster Kidney (BHK) cell, a Human Embryonic Kidney (HEK 293) cell, or an African Green Monkey Kidney (Vero) cell. In some embodiments, the U2OS cell comprises an U2OS cell comprising ICP4 and ICP27 genes (U2OS-4/27). In some embodiments, the U2OS cell comprises an U2OS cell comprising ICP0, ICP4, and ICP27 genes (U2OS-0-4/27).

In some embodiments, the first nucleic acid sequence is introduced into a first genomic safe harbor site of the genome. In some embodiments, the first genomic safe harbor site comprises a Rosa26 homolog locus.

In some embodiments, the second nucleic acid sequence is introduced into a second genomic safe harbor site of the genome. In some embodiments, the second genomic safe harbor comprises an adeno-associated virus site 1 (AAVS1) locus.

In some embodiments, the introducing comprises (i) a recombination mediated cassette exchange; (ii) a homology directed repair; or (iii) a random integration. In some embodiments, (ii) the homology directed repair comprises (a) a homology directed repair following a targeted double strand break generated by an endonuclease or (b) a homology directed repair with long homology arms and naturally occurring double strand breaks. In some embodiments, the endonuclease comprises a Clustered regularly interspaced short palindromic repeats (CRISPR)-CRISPR-associated protein 9 (Cas9), a zinc finger nuclease, or a transcription activator-like effector nuclease (TALEN).

In some embodiments, (b) the homology directed repair with long homology arms and naturally occurring double strand breaks further comprises selection with a marker. In some embodiments, (iii) the random integration further comprises a selection with a marker. In some embodiments, the marker comprises LacZ, CAT, HPRT1, PIGA, Luciferase, GFP, YFP, RFP, iRFP, EGFP, blasticidin, neomycin/G418, hygromycin, puromycin, or zeocin.

In some embodiments, the first nucleic acid sequence further comprises a first one or more regulatory elements. In some embodiments, the one or more HSV Class I genes comprise Infected-Cell Polypeptide 4 (ICP4), ICP27, ICP0, ICP34.5, VP16, or a combination thereof. In some embodiments, the first one or more regulatory elements comprise an enhancer, a promoter, a terminator, or a combination thereof. In some embodiments, the first one or more regulatory elements comprise the promoter. In some embodiments, the promoter comprises an inducible promoter. In some embodiments, the inducible promoter comprises a tetracycline-inducible promoter or a doxycycline-inducible promoter. In some embodiments, the inducible promoter comprises Tet-On promoter comprising TRE3G or TRE-tight.

In some embodiments, the one or more HSV Class II genes comprise UL 1, UL2, UL3, UL4, UL5, UL6, UL7, UL8, UL9, UL10, UL11, UL12, UL13, UL14, UL15, UL16, UL17, UL18, UL19, UL20, UL21, UL22, UL23, UL24, UL25, UL26, UL26.5, UL27, UL28, UL29, UL30, UL31, UL32, UL33, UL34, UL35, UL36, UL37, UL38, UL39, UL40, UL41, UL42, UL43, UL44, UL45, UL46, UL47, UL49, UL49A, UL50, UL51, UL52, UL53, UL55, UL56, US1, US2, US3, US4, US5, US6, US7, US8, US9, US10, US11, US12, or a combination thereof.

In some embodiments, the second nucleic acid sequence further comprises a second one or more regulatory elements. In some embodiments, the second one or more regulatory elements comprise an enhancer, a promoter, a terminator, or a combination thereof. In some embodiments, the second one or more regulatory elements comprise the promoter. In some embodiments, the promoter comprises a natural promoter for HSV Class II genes comprising UL1, UL2, UL3, UL4, UL5, UL6, UL7, UL8, UL9, UL10, UL11, UL12, UL13, UL14, UL15, UL16, UL17, UL18, UL19, UL20, UL21, UL22, UL23, UL24, UL25, UL26, UL26.5, UL27, UL28, UL29, UL30, UL31, UL32, UL33, UL34, UL35, UL36, UL37, UL38, UL39, UL40, UL41, UL42, UL43, UL44, UL45, UL46, UL47, UL49, UL49A, UL50, UL51, UL52, UL53, UL55, UL56, US1, US2, US3, US4, US5, US6, US7, US8, US9, US10, US11, US12, or a combination thereof.

In some embodiments, the genome does not comprise a nucleic acid sequence comprising an HSV origin of replication (ori), an HSV packaging signal (pac), or a combination thereof. In some embodiments, the genome does not comprise a nucleic acid sequence comprising oriL/S.

In some embodiments, the first nucleic acid sequence and/or the second nucleic acid sequence is comprised in a vector. In some embodiments, the vector is selected from the group consisting of a DNA, an RNA, a plasmid, a lentivirus vector, adenoviral vector, a Rous sarcoma viral (RSV) vector, and a retrovirus vector.

### EXAMPLES

These examples are provided for illustrative purposes only and not to limit the scope of the claims provided herein.

### Example 1: Preparation of the Packaging Cell Line

A REWRITE landing pad can be inserted into the Rosa26 homolog locus and the AAVS1 locus of a U2OS cell line. A cassette of HSV Class I genes described herein (ICP4, ICP27, ICP0, ICP34.5, and/or VP16) with an inducible promoter can be inserted to the REWRITE landing pad in the Rosa26 homolog locus. Next, a cassette of HSV Class II genes described herein (UL1, UL2, UL3, UL4, UL5, UL6, UL7, UL8, UL9, UL10, UL11, UL12, UL13, UL14, UL15, UL16, UL17, UL18, UL19, UL20, UL21, UL22, UL23, UL24, UL25, UL26, UL26.5, UL27, UL28, UL29, UL30, UL31, UL32, UL33, UL34, UL35, UL36, UL37, UL38, UL39, UL40, UL41, UL42, UL43, UL44, UL45, UL46, UL47, UL49, UL49A, UL50, UL51, UL52, UL53, UL55, UL56, US1, US2, US3, US4, US5, US6, US7, US8, US9, US10, US11, and/or US12), with their natural promoter, can be inserted into the REWRITE landing pad in the AAVS1 locus.

Using PCR and RNAseq, the expression of HSV Class I and Class II genes can be confirmed. Using single-gene deletion HSV vectors, functional complementation of HSV Class I and Class II genes can be confirmed. For example, plaque formation when HSV vectors lacking either UL 15 or UL17 are transfected into the packaging cell line that encodes HSV Class I and Class II genes including UL 15 and UL 17 can confirm functional complementation of UL 15 or UL17, demonstrating relocating HSV Class I and Class II genes into the genome of the packaging cell line does not affect the expression of HSV Class I and Class II genes.

### Example 2: HSV Amplicon Particles in Immunotherapy

HSV amplicon produced by methods described herein can improve the efficiency of engineering the genome of primary immune cells, including T cells and NK cells, which are both critically important for modern cell therapies. For example, as the field moves towards “off-the-shelf products” and sourcing cells from healthy donors, substantial engineering can be required both to install the therapeutic payload and to overcome immunological barriers and improve engraftment.

More specifically, engineering steps can target two major goals: (1) installation of one or more therapeutic genes (e.g., a chimeric antigen receptors, or CARs); and (2) genetic changes to improve engraftment of therapeutic cells inside the patient (e.g., (a) deleting the T-cell receptor alpha/beta locus (TRAC) to eliminate graft versus host disease, (b) deleting beta-2-microglobulin to eliminate cell surface expression of HLA Class I molecules and eliminate host versus graft disease; and (c) installation of a constitutively expressed HLA-E or HLA-G gene to minimize natural killer cell-mediated loss of therapeutic cells). At least 3 bi-allelic edits encompassing a series of knock-out and knock-in changes can be required. Importantly, the ability to deliver longer payloads via HSV particles described herein can provide several advantages: (1) nuclease system (e.g., TALEN, CRISPR-Cas9, zinc finger) can be encoded genetically together with all of the donor DNAs for homology directed repair (HDR), ensuring that a single transduction event results in co-delivery of all of the sequences required for editing; (2) the HDR DNAs can be substantially longer and encode natural regulatory information and/or introns to enable tissue enhanced transcriptional regulation); and (3) the HSV amplicon system can offer high transduction efficiency and for example, a lower titer may be necessary, compared to lentivirus vector or AAV vector.

### Example 3: Packaging Cell Line Generation and Validation I

### Insertion of UL19, UL20, and UL21 at a REWRITE Landing Pad Located at the AAVS1 Locus (GSH2)

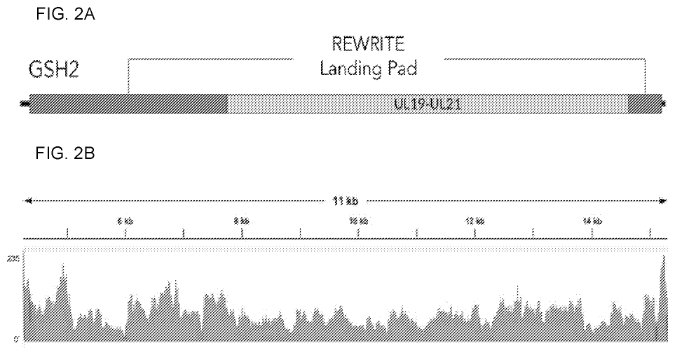

As shown in FIG. 2A, a gene cassette containing HSV class II genes (UL19, UL20, and UL21) flanked by heterotypic Lox sites was constructed in a REWRITE-compatible payload vector (mPAV-UL19-21-Zeo; SEQ ID NO: 17), and delivered to the REWRITE landing pad (SEQ ID NO: 4) at AAVS1 (GSH2) via recombinase mediated cassette exchange (RMCE) in U2OS-4/27 cells. U2OS-4/27 is a packaging cell line stably transduced to express IE175 (ICP4) and UL54 (ICP27) genes (see Miyagawa Y, et al. Herpes simplex viral-vector design for efficient transduction of nonneuronal cells without cytotoxicity Proc Natl Acad Sci USA. 2015 Mar. 31; 112(13): E1632-E1641) and used to complement JDNI8-4 vector (JDNI8-4. HSV-KOS deleted for ICP0, ICP4, ICP27 and JOINT region; see Miyagawa Y, et al. Deletion of the Virion Host Shut-off Gene Enhances Neuronal-Selective Transgene Expression from an HSV Vector Lacking Functional IE Genes. Mol Ther Methods Clin Dev. 2017 Jun. 16; 6:79-90). Briefly, a plasmid containing tamoxifen-induced Cre-recombinase was co-delivered with the payload vector by Nucleofection using the Nucleofector SE Kit (Lonza) as described by the manufacturer's protocol. U2OS-4/27 cells comprising REWRITE Landing Pad were collected by trypsinization, counted, and prepared in the Nucleofection Buffer as suspensions of 1×10⁶ cells per nucleofection condition. DNAs were prepared for co-delivery in equimolar amounts of 0.26 pmol per delivery. Following nucleofection, 1×10⁵ cells were taken from the population and plated in 10 cm dishes as technical duplicates. Cre recombination of the transgenic payload into the GSH landing pad began 24 hours post-nucleofection with the addition of 250 nM Tamoxifen (Sigma) into complete Dubecco's Modified Eagle Media (Gibco) (DMEM, 10% FBS, 1% Pen-strep) for 48 hours. After the recombination period, cell populations were selected in complete DMEM (Gibco) supplemented with 500 μg/ml Zeocin antibiotic (Milipore Sigma) to select for successful integrants.

Following selection for successful integration of the payload containing HSV class II genes (UL19, UL20, and UL21 or UL19-21) at the REWRITE landing pad with Zeocin, U2OS-4/27+UL19-21 cells were pooled, and genomic DNA was extracted. Genomic DNA was used to construct whole-genome sequencing (WGS) libraries using the Illumina DNA library protocol (Illumina, Cat #20060059). In order to enhance the detection of transgene insertions into the cellular genome, biotinylated probes were prepared from transfected plasmids by Nick Translation (Roche) according to the manufacturer's protocol. Probes were assessed for size by resolving on a 2.0% agarose gel. Concentrations of prepared probes were assessed by Qubit HS dsDNA Assay (Thermo). WGS libraries were enriched for the target region by incubation with the prepared biotinylated DNA probes as previously described in Brosh et al, Molecular Cell 83(7):1140-1152 (2023), and the resulting Capture-seq library was purified and sequenced on an Illumina Nextseq 2000 instrument. Capture-seq reads were mapped to the reference Fasta file (AAVS1-UL19-21-insert; SEQ ID NO: 15), and output barn files were used to visualize read coverage over the payload insertion region at AAVS1 in IGV_2.14.1. An exemplary readout of whole-genome sequencing (WGS) after enrichment for sequences (Capture-seq) at the AAVS1 genome and sequences contained in the synHSV93 genome in the U2OS-4/27+UL19-21 cells was shown in FIG. 2B.

This experiment demonstrates the successful preparation of U2OS-4/27+UL19-21 packaging cells.

### Complementation of ICP4 (IE175 Gene) and ICP27 (UL54 Gene) in U2OS-4/27+UL19-21 Cells

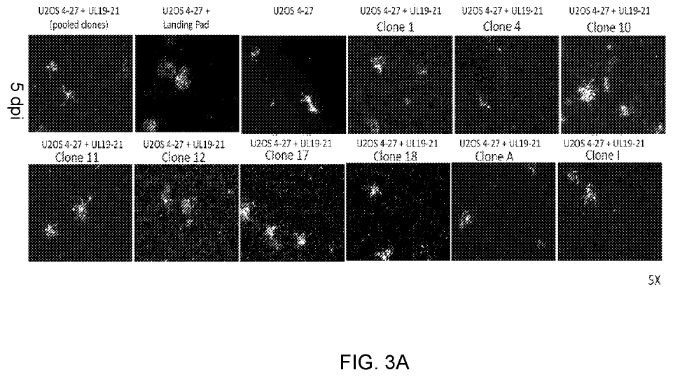

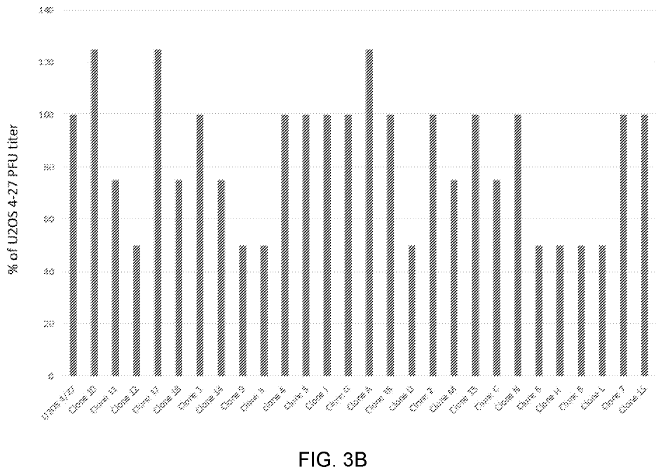

Pooled clones of U2OS-4/27+UL19-21 cells were replated at clonal density, and individual clones were picked and expanded in culture. U2OS-4/27 cells, U2OS-4/27+REWRITE Landing Pad cells, U2OS-4/27+UL19-21 pooled clones, and U2OS4/27+UL19-21 individual clones were plated in 48-well plates (4×10⁴ cells/well) and subsequently infected with the replication-deficient JDNI8-4 HSV vector at multiplicity of infection (MOI) of 0.0005 pfu/cell. Representative fluorescent images of JDNI8-4 plaques for each cell line at 5 days post-infection (dpi) were obtained with a Nikon Diaphot fluorescence microscope (Nikon) at 5× magnification, as shown in FIG. 3A.

Next, U2OS-4/27 and U2OS-4/27+UL19-21 clones were plated in 48-well plates and infected with serial dilutions of JDNI8-4 HSV vector. Two hours post-infection the cells were overlayed with methylcellulose. Titer was determined by plaque count at 9 dpi and normalized to the titer obtained in the U2OS-4/27 packaging line. See FIG. 3B.

These experiments demonstrate that U2OS-4/27+UL19-21 cells package JDNI8-4 HSV virus vector with similar efficiency to parental U2OS-4/27 cells.

### Complementation of VP 5 (UL19 Gene) in U2OS-4/27+UL19-21 Cells

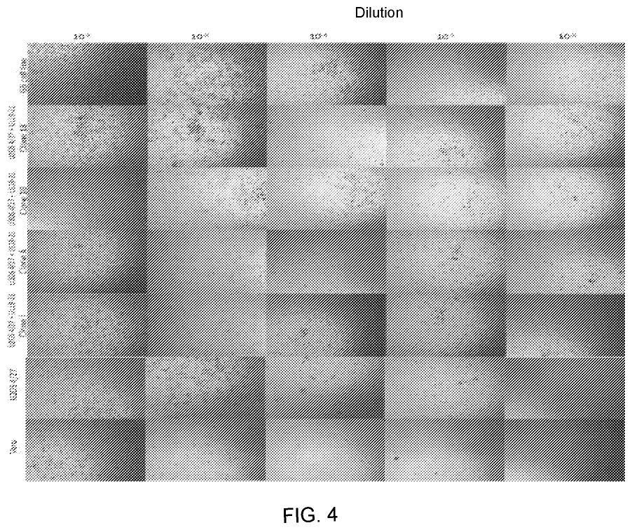

Complementation of VP5 (UL19 gene) in U2OS-4/27+UL19-21 cells was tested by infection with a replication competent virus deleted for UL19 gene (ΔVP5; HSV-KOS deleted for VP5/UL19 gene; see Desai P, et al. Mutations in herpes simplex virus type 1 genes encoding VP5 and VP23 abrogate capsid formation and cleavage of replicated DNA. J Virol. 1993 March; 67(3):1357-64). U2OS-4/27+UL19-21 clones, Vero G5 (packaging cell line stably transduced to express UL16-UL21 HSV genes and used to complement ΔVP5 virus), U2OS-4/27, and Vero (negative controls) were plated in 48-well plates (4×10⁴ cells/well). Cells were infected with serial dilution of ΔVP5 HSV virus. Representative images of ΔVP5 HSV virus plaques were taken at 2 dpi with a Nikon Diaphot microscope (Nikon) at 5× magnification as shown in FIG. 4.

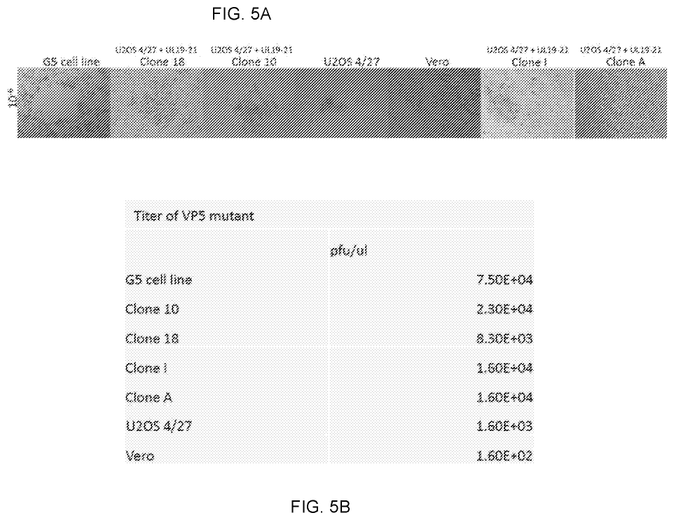

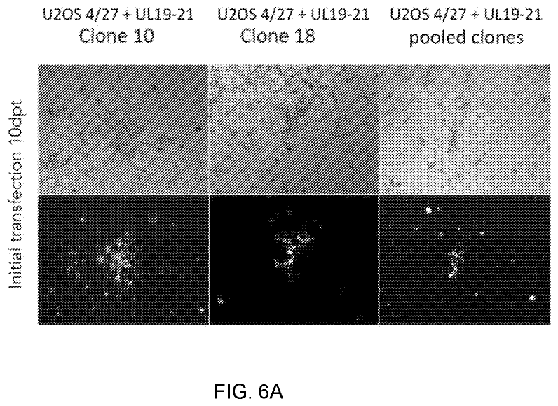

ΔVP5 HSV virus pfu titer in U2OS-4/27+UL19-21 clones and Vero G5 cells were compared, and results are shown in FIGS. 5A and 5B.

This experiment demonstrates that U2OS-4/27+UL19-21 cells package ΔVP5 HSV virus with similar efficiency to Vero G5 cells.

### Complementation of VP5 (UL19 Gene), UL20p (UL20 Gene), and UL21 (UL21 Gene) in U2OS-4/27+UL19-21 Clones

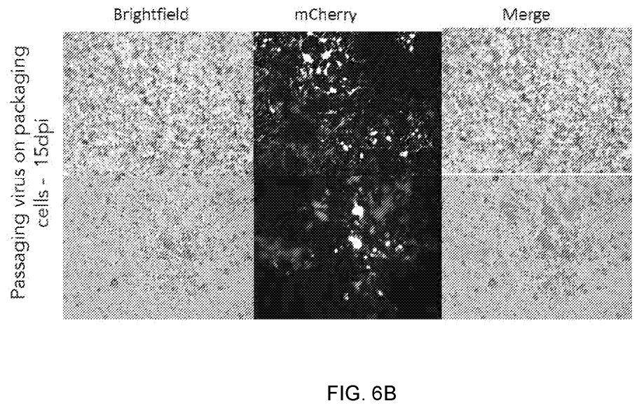

Complementation of VP5 (UL19 gene), UL20p (UL20 gene), and UL21 (UL21 gene) in U2OS-4/27+UL19-21 cells was tested by infection with a replication deficient mutant HSV vector additionally deleted for UL19-UL21 genes. JDNI8-ΔUL19-UL21 (SEQ ID NO: 11). U2OS-4/27+UL19-21 clones were plated in 6-well plates at (3×10⁵ cell/well) and transfected with 2 μg of JDNI8-AUL19-UL21 vector DNA. Representative fluorescent pictures of JDNI8-ΔUL19-UL21 plaques taken 10 days post-transfection (dpt) are shown FIG. 6A. Pictures were obtained with a Nikon Diaphot fluorescence microscope (Nikon) at 5× magnification (upper panel, brightfield; lower panel, mCherry).

For propagation of the JDNI8-ΔUL19-UL21 vector on U2OS-4/27+UL19-21 clones, cells were plated in 6-well plates and infected with supernatant recovered from U2OS-4/27+UL19-21 transfected with JDNI8-ΔUL19-UL21 vector. FIG. 6B shows a representative picture of U2OS-4/27+UL19-21 clone 18 infection at 15 dpi.

This experiment demonstrates that U2OS-4/27+UL19-21 cells package JDNI8-ΔUL19-UL21 HSV virus efficiently, and the resulting HSV amplicon particles can be used for re-passaging to increase titer.

### Example 4: Packaging Cell Line Generation and Validation II

Insertion of synHSV128 Genome at a REWRITE Landing Pad Located at the AAVS1 Locus (GSH2)

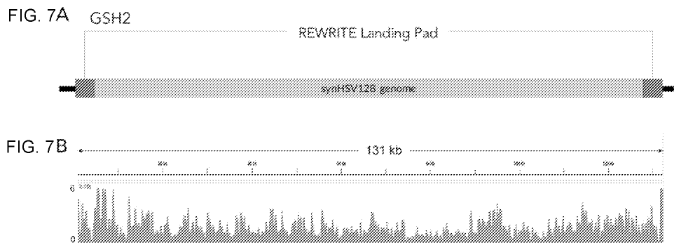

A gene cassette containing HSV class I and II genes was constructed in a REWRITE-compatible payload vector (synHSV128_Puro; SEQ ID NO: 9). synHSV128_Puro vector comprises HSV II genes, UL19-UL56 and US1-US11 under their native promoters; HSV I genes, VP16 under TRE3GS promoter and ICP34.5 under a CMV promoter; Yellow Fluorescent Protein (YFP) gene; and Puromycin resistance gene, synHSV128_Puro vector does not comprise on or pac sequences. The DNA payload was delivered to the REWRITE landing pad (SEQ ID NO: 4) at AAVS1 (GSH2) in U2OS cells by co-transfection with a plasmid containing a tamoxifen-induced Cre-recombinase to induce recombinase mediated cassette exchange (RMCE) integration. DNAs were prepared for transfection in equimolar amounts at 0.26 pmol per DNA. 24 hours prior to transfection, 2×10⁵ cells were plated into 6-well plate format. Co-transfection was performed using the FuGene HD reagent in a 1:1 reagent to total DNA volumetric ratio. 24 Hours post transfection, cells were treated with 250 nM tamoxifen to induce Cre-driven recombination at the REWRITE Landing Pad site. Acute exposure to tamoxifen was carried out for 48 hours. Following the recombination period, cell populations were given an additional 48 hours to recover in complete DMEM. Following the recovery period, antibiotic selection was performed for 14 days in DMEM supplemented with 1 μg/ml puromycin. A schematic drawing of the synthetic genome integrated at GSH2 is shown in FIG. 7A.

Following selection for successful integration of the payload at the REWRITE landing pad with puromycin, single cell derived clones of U2OS+synHSV128 cells were isolated and propagated. For screening and cell line characterization, genomic DNA was extracted using the DNEasy Blood and Tissue Kit (Qiagen). Genomic DNA was then used to construct whole-genome sequencing (WGS) libraries using the Integrated DNA Technologies ExGen DNA EZ Kit (IDT. Cat #10009863). WGS libraries were enriched for the target region using biotinylated DNA probes as previously described. The resulting Capture-seq libraries were sequenced on an Illumina Nextseq 2000 instrument. Capture-seq reads were mapped to the reference Fasta file (AAVS1-synHSV128-insert; SEQ ID NO: 16), and output bam files were used to visualize read coverage over the payload insertion region at AAVS1 in IGV_2.14.1. An exemplary readout of whole-genome sequencing (WGS) after enrichment for sequences (Capture-seq) at the AAVS1 genome and sequences contained in the synHSV128 genome in U2OS+synHSV128 is shown in FIG. 7B.

Complementation of Replication Competent HSV Viruses with synHSV128 Packaging Cells

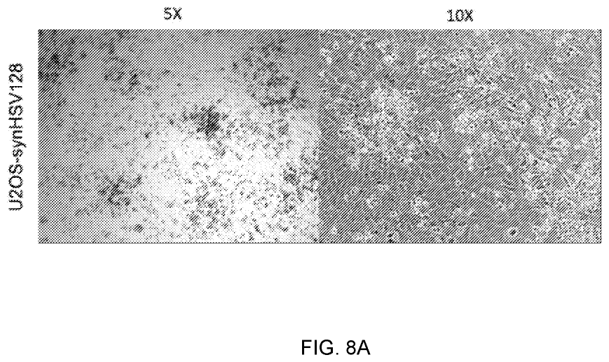

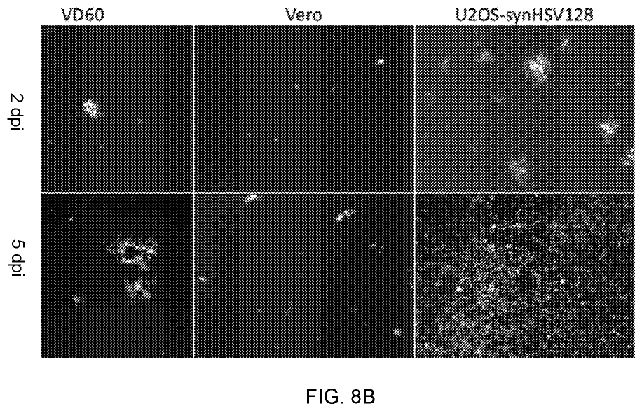

Complementation of a ΔVP5 (UL19) HSV replication competent vector virus (ΔVP5; HSV-KOS deleted for VP5 gene) in U2OS+synHSV128 packaging cells was tested. U2OS+synHSV128 packaging cells were plated in 48-well plates and infected with serial dilution of ΔVP5 HSV virus. Representative pictures of ΔVP5 HSV virus plaques were taken at 7 dpi are shown in FIG. 8A.

Next, complementation of a ΔgD (US6 gene) replication competent virus (ΔgD; HSV-KOS deleted for gD/US6 gene; see Ligas M W, Johnson DC. A herpes simplex virus mutant in which glycoprotein D sequences are replaced by beta-galactosidase sequences binds to but is unable to penetrate cells. J Virol. 1988 May; 62(5):1486-94) in Vero VD60 (ΔgD virus packaging cells, a cell line stably transduced to express gD/US6 gene), U2OS-synHSV128, and Vero cells (negative control) was tested. ΔgD virus was grown on VD60 cells upon transfection. Transfection supernatant was collected at 9 days post transfection and serial dilutions of the supernatant were used to infect Vero VD60 (ΔgD virus packaging cell line), U2OS-synHSV128, and Vero cells. Representative images were captured at 2 dpi and 5 dpi as shown in FIG. 8B.

This experiment demonstrates that U2OS-synHSV128 cells package replication defective HSV viruses ΔVP5 and ΔgD efficiently.

### Example 5: Packaging Cell Line Generation and Validation III

A vector comprising human codon-optimized Immediate Early Genes ICP0, ICP4, and ICP27 genes under the control of the TRE-tight promoter (TRE-ICP0-4-27; SEQ ID NO: 8) can be constructed. This construct can be then prepared for genomic integration by introducing homology arms targeting the syntenic human Rosa26 locus in 3 different cell lines: 1) U2OS cells comprising the REWRITE landing pad at GSH2; 2) U2OS-4/27+UL19-21 (U2OS-4/27 cell line comprising UL19-21 genes inserted in landing pad at GSH2; see Example 3), and 3) U2OS+synHSV128 (U2OS cell line comprising synHSV128 integrated at GSH2; see Example 4). Genomic integrations of TRE-ICP0-4/27 can be performed by co-nucleofection of the payload plasmid with a Cas9-expressing plasmid containing a guide RNA sequence corresponding to an intronic sequence within the ROSA26 locus. Co-nucleofection of DNAs can be performed using the Nucleofector SE Kit (Lonza) as described by the manufacturer's protocol. Cells can be collected by trypsinization, counted, and prepared in the provided Nucleofection Buffer as suspensions of 1×10⁶ cells per nucleofection condition. DNAs can be prepared for co-delivery in equal amounts of 2 μg per delivery. In addition, a single-stranded oligodeoxynucleotide (ssODN) can be added at a concentration of 200 nM. Following nucleofection, 1×10⁵ cells can be taken from the population and plated in 10 cm dishes as technical duplicates. Cell populations can be selected in complete DMEM (10% FBS, 1% P/S)+1 μg/ml puromycin to enrich for those cells within the population that take up the Cas9 plasmid. Puromycin selective pressure can be applied for 48 hours. After 48 hours selective pressure can be changed to neomycin (G418) (500 Dg/ml) to enrich for the presence of the TRE-ICP0-4/27 integration. Selection with G418 can be carried out for 10 days. Genomic DNA from the cell line can be extracted using the Qiagen DNAEasy Kit (Qiagen). Pooled transfectant genomic DNA samples can be screened for integration of TRE-ICP0/4/27 by PCR.

The resulting U2OS-0-4/27 cells can be tested for complementation of HSV vectors, e.g., JDN18-4, JDNI8-ΔUL19-21 (SEQ ID NO:11), and/or SEQ ID NO: 13, by infecting the cells with the HSV vector. U2OS-0-4/27 clones can be plated in 6-well plates at (3×10⁵ cell/well) and transfected with 2 μg of the HSV vector DNA.

For propagation of the HSV vector on U2OS-0-4/27 clones, cells will be plated in 6-well plates and infected with supernatant recovered from U2OS-0-4/27 transfected with the HSV vector.

It is expected that that U2OS-0-4/27 cells will package the HSV virus efficiently, and the resulting HSV amplicon particles can be used for re-passaging to increase titer.

### Example 6: Sequence Listing

TABLE 1
Description of Sequences

| SEQ | | |
|---|---|---|
| ID | | |
| NO: | Name of sequence | Description |
| 1 | AmpliconVector_synHSV_{—} | An amplicon vector comprising |
| | 93 | HSV Class II genes UL1-UL19, |
| | | HSV Class I gene VP16, |
| | | mCherry, ori, and pac |
| | | sequences. |
| 2 | GSH1_ClassI_v1_TRE- | A construct integrated at GSH 1 |
| | ICP0-4-27 | via Cas9-mediated double strand |
| | | break (DSB), comprising a tet- |
| | | inducible (TRE3GS promoter) |
| | | gene cassette that drives |
| | | expression of codon-optimized |
| | | ICP0, ICP4, and ICP27 genes on |
| | | a single transcript, separated by |
| | | P2A cleavage signals. |
| 3 | GSH1_ClassI_v2_TRE- | A construct integrated at GSH 1 |
| | ERT2-ICP0-4-27 | via Cas9-mediated double strand |
| | | break (DSB), comprising a tet- |
| | | inducible (TRE promoter) gene |
| | | cassette that drives expression of |
| | | codon-optimized ICP0-ERT2, |
| | | ICP4-ERT2, and ICP27-ERT2 |
| | | genes on a single transcript, |
| | | separated by P2A cleavage |
| | | signals. |
| 4 | GSH2_ClassII_{—} | Landing pad sequence |
| | LandingPadSequence | |
| 5 | GSH2_ClassII_synHSV_{—} | A synthetic HSV genome for |
| | 93 | RMCE integration at the landing |
| | | pad (SEQ ID NO: 4) at GSH2 for |
| | | construction of the packaging |
| | | cell line; comprises HSV class I |
| | | gene ICP34.5 driven by a CMV |
| | | promoter; Class II genes UL19- |
| | | UL56, US1-US11 with their |
| | | native promoters; YFP gene; |
| | | Zeocin resistance gene; does not |
| | | comprise ori/pac sequences. |
| 6 | AmpliconVector_additional_{—} | A construct comprising HSV |
| | HSV_ClassII | class I gene ICP34.5 with its |
| | | native promoter; Class II genes |
| | | UL19-UL56, US1-US11 with |
| | | their native promoters; does not |
| | | comprise ori/pac sequences. |
| 7 | TRE-Tight-0-4-27-VP16- | A construct integrated at GSH 1 |
| | ROSA26HAs | via Cas9-mediated double strand |
| | | break (DSB), comprising: a tet- |
| | | inducible (TRE-tight promoter) |
| | | gene cassette that drives |
| | | expression of codon-optimized |
| | | ICP0, ICP4, and ICP27 genes on |
| | | a single transcript, separated by |
| | | P2A cleavage signals; a tet- |
| | | inducible VP16 gene cassette; |
| | | and homology arms to target |
| | | integration of these set-inducible |
| | | genes to the Rosa26 locus. This |
| | | construct can be used for the |
| | | generation of the packaging cell |
| | | line. |
| 8 | TRE-Tight-0-4-27_{—} | A construct integrated at GSH 1 |
| | ROSA26_HomologyArm_{—} | via Cas9-mediated double strand |
| | Assembly | break (DSB), comprising: a tet- |
| | | inducible (TRE-tight promoter) |
| | | gene cassette that drives |
| | | expression of codon-optimized |
| | | ICP0, ICP4, and ICP27 genes on |
| | | a single transcript, separated by |
| | | P2A cleavage signals; and |
| | | homology arms to target |
| | | integration of these set-inducible |
| | | genes to the Rosa26 locus. This |
| | | construct can be used for the |
| | | generation of the packaging cell |
| | | line. |
| 9 | SynHSV128_Puro | A synthetic HSV genome for |
| | | RMCE integration at the landing |
| | | pad (SEQ ID NO: 4) at GSH2 for |
| | | construction of the packaging |
| | | cell line. This synthetic HSV |
| | | genome comprises HSV class I |
| | | genes (TRE3GS-VP16, and |
| | | ICP34.5 driven by a CMV |
| | | promoter); Class II genes (UL19- |
| | | UL56 and US1-US11 under |
| | | native promoter); YFP gene; |
| | | Puromycin resistance gene; and |
| | | does not comprise ori/pac |
| | | sequences. |
| 10 | SynHSV128_ΔVP16 | A synthetic HSV genome for |
| | | RMCE integration at the landing |
| | | pad (SEQ ID NO: 4) at GSH2 for |
| | | construction of the packaging |
| | | cell line. This synthetic HSV |
| | | genome comprises HSV class I |
| | | gene (ICP34.5 driven by a CMV |
| | | promoter); Class II genes (UL19- |
| | | UL56 and US1-US11 under |
| | | native promoter); YFP gene; |
| | | Puromycin resistance gene; and |
| | | does not comprise ori/pac |
| | | sequences. |
| 11 | JDNI8-ΔUL19-21 | A replication-deficient HSV |
| | | vector comprising deletion of |
| | | UL19/UL20/UL21 genes. This |
| | | vector has capacity for 45 kb of |
| | | DNA payload, and can be |
| | | transfected into a packaging cell |
| | | line to produce replication- |
| | | deficient virus. |
| 12 | JDNI5-ΔUL19-21 | A replication-deficient HSV |
| | | vector comprising deletion of |
| | | UL19/UL20/UL21/UL41 genes. |
| | | This vector has capacity for 45 kb |
| | | of DNA payload, and can be |
| | | transfected into a packaging cell |
| | | line to produce replication- |
| | | deficient virus. |
| 13 | AmpVec_VP16-4-27 | An amplicon vector comprising |
| | | VP16 driven by EF1alpha |
| | | promoter; ICP4 and ICP27 on |
| | | their native promoters; pac/ori |
| | | sequences; Flag-tagged |
| | | Luciferase driven by CAG |
| | | promoter; and GFP driven by |
| | | the oriS + IE4/5 promoter. |
| 14 | AmpVec_4-27 | An amplicon vector comprising |
| | | ICP4 and ICP27 on their native |
| | | promoters; pac/ori sequences; |
| | | Flag-tagged Luciferase driven by |
| | | CAG promoter; and GFP driven |
| | | by the oriS + IE4/5 promoter. |
| 15 | AAVS1-UL19-21-insert | AAVS1-UL19-21-insert |
| | | reference sequence used for |
| | | mapping (Example 3) |
| 16 | AAVS1-synHSV128-insert | AAVS1-synHSV128-insert |
| | | reference sequence used for |
| | | mapping (Example 4) |
| 17 | mPAV-UL19-21-Zeo | A vector comprising HSV class |
| | | II genes (UL19, UL20, and |
| | | UL21) flanked by heterotypic |
| | | Lox sites, Zeocin resistance gene, |
| | | and YFP gene for integration into |
| | | GSH2 |

What is claimed is:

1. A packaging cell line comprising a recombinant genome, wherein the recombinant genome comprises:
a. a first nucleic acid sequence comprising one or more Herpes Simplex Virus (HSV) Class I genes; and
b. a second nucleic acid sequence comprising one or more HSV Class I and Class II genes.

2. The packaging cell line of claim 1, wherein the first nucleic acid sequence further comprises a first one or more regulatory elements.

3. The packaging cell line of claim 1, wherein the first nucleic acid sequence is in a first genomic safe harbor site of the recombinant genome.

4. The packaging cell line of claim 3, wherein the first genomic safe harbor site comprises a Rosa26 homolog locus.

5. The packaging cell line of claim 1, wherein the one or more HSV Class I genes comprise Infected-Cell Polypeptide 4 (ICP4), ICP27, ICP0, ICP34.5, VP16, or a combination thereof.

6. The packaging cell line of claim 2, wherein the first one or more regulatory elements comprise an enhancer, a promoter, a terminator, or a combination thereof.

7. The packaging cell line of claim 6, wherein the first one or more regulatory elements comprise the promoter.

8. The packaging cell line of claim 7, wherein the promoter comprises an inducible promoter.

9. The packaging cell line of claim 8, wherein the inducible promoter comprises a tetracycline-inducible promoter or a doxycycline-inducible promoter.

10. The packaging cell line of claim 9, wherein the inducible promoter comprises Tet-On promoter comprising TRE3G or TRE-tight.

11. The packaging cell line of claim 1, wherein the second nucleic acid sequence is in a second genomic safe harbor site of the recombinant genome.

12. The packaging cell line of claim 11, wherein the second genomic safe harbor comprises an adeno-associated virus site 1 (AAVS1) locus.

13. The packaging cell line of claim 1, wherein the one or more HSV Class II genes comprise UL1, UL2, UL3, UL4, UL5, UL6, UL7, UL8, UL9, UL10, UL 11, UL12, UL13, UL14, UL15, UL16, UL17, UL18, UL19, UL20, UL21, UL22, UL23, UL24, UL25, UL26, UL26.5, UL27, UL28, UL29, UL30, UL31, UL32, UL33, UL34, UL35, UL36, UL37, UL38, UL39, UL40, UL41, UL42, UL43, UL44, UL45, UL46, UL47, UL49, UL49A, UL50, UL51, UL52, UL53, UL55, UL56, US1, US2, US3, US4, US5, US6, US7, US8, US9, US10, US11, US12, or a combination thereof.

14. The packaging cell line of claim 1, wherein the second nucleic acid sequence further comprises a second one or more regulatory elements.

15. The packaging cell line of claim 14, wherein the second one or more regulatory elements comprise an enhancer, a promoter, a terminator, or a combination thereof.

16. The packaging cell line of claim 15, wherein the second one or more regulatory elements comprise the promoter.

17. The packaging cell line of claim 16, wherein the promoter comprises a natural promoter for HSV Class II genes comprising UL1, UL2, UL3, UL4, UL5, UL6, UL7, UL8, UL9, UL10, UL11, UL12, UL13, UL14, UL15, UL16, UL17, UL18, UL19, UL20, UL21, UL22, UL23, UL24, UL25, UL26, UL26.5, UL27, UL28, UL29, UL30, UL31, UL32, UL33, UL34, UL35, UL36, UL37, UL38, UL39, UL40, UL41, UL42, UL43, UL44, UL45, UL46, UL47, UL49, UL49A, UL50, UL51, UL52, UL53, UL55, UL56, US1, US2, US3, US4, US5, US6, US7, US8, US9, US10, US11, US12, or a combination thereof.

18. The packaging cell line of claim 1, wherein the recombinant genome does not comprise a nucleic acid sequence comprising an HSV origin of replication (ori), an HSV packaging signal (pac), or a combination thereof.

19. The packaging cell line of claim 1, wherein the recombinant genome does not comprise a nucleic acid sequence comprising oriL/S.

20. The packaging cell line of claim 1, wherein the first nucleic acid sequence comprises a sequence with at least 50% sequence identity to SEQ ID NO: 2, SEQ ID NO: 3, SEQ ID NO: 7, or SEQ ID NO: 8.

21. The packaging cell line of claim 20, wherein the first nucleic acid sequence comprises a sequence comprising SEQ ID NO: 2, SEQ ID NO: 3, SEQ ID NO: 7, or SEQ ID NO: 8.

22. The packaging cell line of claim 1, wherein the second nucleic acid sequence comprises a sequence with at least 50% sequence identity to SEQ ID NO: 5, SEQ ID NO: 6, SEQ ID NO: 9, SEQ ID NO: 10, or SEQ ID NO: 17.

23. The packaging cell line of claim 22, wherein the second nucleic acid sequence comprises a sequence comprising SEQ ID NO: 5, SEQ ID NO: 6, SEQ ID NO: 9, SEQ ID NO: 10, or SEQ ID NO: 17.

24. The packaging cell line of claim 1, wherein the packaging cell line comprises a mammalian cell line.

25. The packaging cell line of claim 24, wherein the packaging cell line is a Human Bone Osteosarcoma Epithelial (U2OS) cell line, a Baby Hamster Kidney (BHK) cell line, a Human Embryonic Kidney (HEK 293) cell line, or an African Green Monkey Kidney (Vero) cell line.

26. The packaging cell line of claim 25, wherein the U2OS cell line comprises U2OS cells comprising ICP4 and ICP27 genes (U2OS-4/27).

27. The packaging cell line of claim 25, wherein the U2OS cell line comprises U2OS cells comprising ICP0, ICP4, and ICP27 genes (U2OS-0-4/27).

28. A system for producing a Herpes Simplex Virus (HSV) amplicon particle comprising:
a. the packaging cell line of any one of claims 1-27; and
b. an amplicon vector.

29. The system of claim 28, wherein the amplicon vector comprises at least one cargo nucleic acid sequence.

30. The system of claim 29, wherein the at least one cargo nucleic acid sequence comprises from about 10 to about 150 kb.

31. The system of claim 29, wherein the at least one cargo nucleic acid sequence comprises up to 100 kb.

32. The system of any one of claims 29-31, wherein the at least one cargo nucleic acid sequence is inserted site-specifically in the amplicon vector by restriction enzyme digestion cloning, Golden Gate Assembly, in vitro Cas9 digestion followed by Gibson assembly, Gateway cloning, or in yeasto recombination assembly.

33. The system of claim 32, wherein the at least one cargo nucleic acid sequence is inserted by restriction enzyme digestion cloning using one or more restriction enzyme sites comprising PacI, BbsI, BbsI-HF, BcoDI, BsaI-HF, BsmBI-v2, BsmI, BspQI, BsrDI, BsrI, BtsI-v2, BtsIMutI, EarI, Esp3I, SapI, AcuI, BaeI, BcgI, BpmI, BpuEI, BsaXI, BseRI, BsgI, BsmFI, BspCNI, BtgZI, CspCI, EcI, I, MmeI, NmeAIII, AwI, BbvI, BccI, BceAI, BcI, VI, BfuAI, BmrI, BsmAI, BspMI, BtsCI, FauI, FokI, HgaI, HphI, HpyAV, MboII, MlyI, MnlI, PaqCI, PleI, SfaNI, I-CeuI, I-SceI, PI-PspI, PI-SceI, PmeI, or a combination thereof.

34. The system of claim 32, wherein the at least one cargo nucleic acid sequence is inserted by Gateway cloning using one or more Gateway cloning sites comprising attR1, attR2, or a combination thereof.

35. The system of claim 32, wherein the at least one cargo nucleic acid sequence is inserted by in yeasto recombination assembly using one or more in yeasto recombination sites comprising NeoLVec, NeoRVec, or a combination thereof.

36. The system of claim 32, wherein the at least one cargo nucleic acid sequence is inserted by in vitro Cas9 digestion followed by Gibson assembly using a Cas9 gRNA sequence.

37. The system of any one of claims 29-36, wherein the at least one cargo nucleic acid sequence comprises a transgene.

38. The system of claim 37, wherein the transgene comprises CRISPR associated protein 9 (Cas9), Cas 3, Cas12a, Dystrophin (DYS), amylo-alpha-1, 6-glucosidase, 4-alpha-glucanotransferase (AGL), otoferlin (OTOF), myosin VIIA (MYO7A), ATP binding cassette subfamily A member 4 (ABCA4), coagulation factor VIII (F8), centrosomal protein 290 (CEP290), cadherin related 23 (CDH23), or centrosome and basal body associated protein (ALMS1).

39. The system of claim 37, wherein the transgene comprises a gene encoding an agent for treating a cancer, muscular dystrophy, non-syndromic deafness, Stargardt Disease, Leber congenital amaurosis 10, Alstrom syndrome, a glycogen storage disease, Usher Syndrome 1D, Usher Syndrome 1B, a cardiovascular disease, a neurodegenerative disease, chronic pain, a lung disease, or hemophilia.

40. The system of any one of claims 29-39, wherein the at least one cargo nucleic acid sequence further comprises at least one regulatory element comprising an enhancer, a promoter, a terminator, or a combination thereof.

41. The system of any one of claims 28-40, wherein the amplicon vector comprises an origin of replication (ori) and a packaging signal (pac).

42. The system of any one of claims 28-41, wherein the HSV amplicon particle comprises an HSV-1 or an HSV-2 amplicon particle.

43. The system of any one of claims 28-42, wherein the amplicon vector comprises a sequence with at least 50% sequence identity to SEQ ID NO: 1, SEQ ID NO: 11, SEQ ID NO: 12, SEQ ID NO: 13, or SEQ ID NO: 14.

44. The system of any one of claims 28-43, wherein the amplicon vector comprises a sequence comprising SEQ ID NO: 1, SEQ ID NO: 11, SEQ ID NO: 12, SEQ ID NO: 13, or SEQ ID NO: 14.

45. A method of producing a Herpes Simplex Virus (HSV) amplicon particle comprising transfecting the packaging cell line of any one of claims 1-44 with an amplicon vector.

46. The method of claim 45, further comprising isolating the HSV amplicon particle from the packaging cell line.

47. The method of claim 45 or 46, wherein the HSV amplicon particle comprises an HSV-1 or an HSV-2 amplicon particle.

48. The method of any one of claims 45-47, wherein the amplicon vector comprises an origin of replication (ori) and a packaging signal (pac).

49. The method of any one of claims 45-48, wherein the amplicon vector further comprises at least one cargo nucleic acid sequence.

50. The method of claim 49, wherein the at least one cargo nucleic acid sequence comprises a transgene.

51. The method of claim 50, wherein the transgene comprises CRISPR associated protein 9 (Cas9), Cas3, Cas12a, Dystrophin (DYS), amylo-alpha-1, 6-glucosidase, 4-alpha-glucanotransferase (AGL), otoferlin (OTOF), myosin VIIA (MYO7A), ATP binding cassette subfamily A member 4 (ABCA4), coagulation factor VIII (F8), centrosomal protein 290 (CEP290), cadherin related 23 (CDH23), or centrosome and basal body associated protein (ALMS1).

52. The method of claim 50, wherein the transgene comprises a gene encoding an agent for treating a cancer, muscular dystrophy, non-syndromic deafness, Stargardt Disease, Leber congenital amaurosis 10, Alstrom syndrome, a glycogen storage disease, Usher Syndrome 1D, Usher Syndrome 1B, a cardiovascular disease, a neurodegenerative disease, chronic pain, a lung disease, or hemophilia.

53. The method of any one of claims 49-52, wherein the at least one cargo nucleic acid sequence further comprises at least one regulatory element comprising an enhancer, a promoter, a terminator, or a combination thereof.

54. The method of any one of claims 45-53, wherein the amplicon vector comprises a sequence with at least 50% sequence identity to SEQ ID NO: 1, SEQ ID NO: 11, SEQ ID NO: 12, SEQ ID NO: 13, or SEQ ID NO: 14.

55. The method of any one of claims 45-54, wherein the amplicon vector comprises a sequence comprising SEQ ID NO: 1, SEQ ID NO: 11, SEQ ID NO: 12, SEQ ID NO: 13, or SEQ ID NO: 14.

56. A Herpes Simplex Virus (HSV) amplicon particle produced by the method of any one of claims 45-55.

57. The HSV particle of claim 56, further comprising HSV capsid, tegument, and envelope proteins.

58. A pharmaceutical composition comprising the HSV amplicon particle of claim 56 or 57, and a pharmaceutically acceptable carrier.

59. A cell comprising the HSV amplicon particle of claim 56 or 57.

60. A kit comprising the HSV amplicon particle of claim 56 or 57, or the packaging cell line of any one of claims 1-27 and an instruction manual for using the kit.

61. A kit for preparing an HSV amplicon particle comprising (i) the packaging cell line of any one of claims 1-27 and (ii) an amplicon vector comprising an origin of replication (ori) and a packaging signal (pac); and an instruction manual for using the kit.

62. The kit of claim of 61, wherein the amplicon vector further comprises at least one cargo sequence.

63. The kit of claim of 62, wherein the at least one cargo sequence comprises a transgene selected from the group consisting of CRISPR associated protein 9 (Cas9), Cas3, Cas12a, Dystrophin (DYS), amylo-alpha-1, 6-glucosidase, 4-alpha-glucanotransferase (AGL), otoferlin (OTOF), myosin VIIA (MYO7A), ATP binding cassette subfamily A member 4 (ABCA4), coagulation factor VIII (F8), centrosomal protein 290 (CEP290), cadherin related 23 (CDH23), or centrosome and basal body associated protein (ALMS1).

64. The kit of claim 62, wherein the at least one cargo sequence comprises a transgene encoding an agent for treating a cancer, muscular dystrophy, non-syndromic deafness, Stargardt Disease, Leber congenital amaurosis 10, Alstrom syndrome, a glycogen storage disease, Usher Syndrome 1D, Usher Syndrome 1B, a cardiovascular disease, a neurodegenerative disease, chronic pain, a lung disease, or hemophilia.

65. The kit of any one of claims of 62-64, wherein the at least one cargo nucleic acid sequence further comprises at least one regulatory element comprising an enhancer, a promoter, a terminator, or a combination thereof.

66. The kit of any one of claims 60-65, wherein the amplicon vector comprises a sequence with at least 50% sequence identity to SEQ ID NO: 1, SEQ ID NO: 11, SEQ ID NO: 12, SEQ ID NO: 13, or SEQ ID NO: 14.

67. The kit of any one of claims 60-66, wherein the amplicon vector comprises a sequence comprising SEQ ID NO: 1, SEQ ID NO: 11, SEQ ID NO: 12, SEQ ID NO: 13, or SEQ ID NO: 14.

68. A method of expressing a transgene in a cell, the method comprising contacting the cell with an HSV amplicon particle produced by the method of any one of claims 45-55, or the HSV amplicon particle of claim 56 or 57.

69. The method of claim 68, wherein the contacting occurs in vivo.

70. The method of claim 68 or 69, wherein the cell is a liver cell, a lung cell, an epithelial cell, a neural cell, a cardiac cell, a muscle cell, a stem cell, or a cancer cell.

71. The method of claim 68, wherein the contacting occurs in vitro.

72. The method of any one of claims 68-71, wherein the cell is a human cell.

73. A method of treating a disease or condition in a subject in need thereof, the method comprising administering to the subject therapeutically effective amount of a Herpes Simplex Virus (HSV) amplicon particle produced by the method of any one of claims 45-55, the HSV amplicon particle of claim 56 or 57, or the pharmaceutical composition of claim 58, wherein the therapeutically effective amount comprises an amount sufficient to infect cells of the subject such that at least one transgene is expressed in the subject.

74. The method of claim 73, wherein the disease or condition comprises a cancer, muscular dystrophy, non-syndromic deafness, Stargardt Disease, Leber congenital amaurosis 10, Alstrom syndrome, a glycogen storage disease, Usher Syndrome 1D, Usher Syndrome 1B, a cardiovascular disease, a neurodegenerative disease, chronic pain, a lung disease, or hemophilia.

75. The method of claim 73, wherein the disease or condition comprises a disease or condition caused by a genetic mutation.

76. The method of claim 74, wherein the disease or condition is a cancer.

77. The method of claim 76, wherein the at least one transgene encodes an agent that enhances tumor killing activity.

78. The method of any one of claims 73-77, wherein the subject is a human.

79. A use of the packaging cell line of any one of claims 1-27 or the system of any one of claims 28-44 for producing a Herpes Simplex Virus (HSV) amplicon particle.

80. The use of claim 79, wherein the producing comprises transfecting the packaging cell line with an amplicon vector.

81. The use of claim 79 or 80, wherein the producing further comprises isolating the HSV amplicon particle from the packaging cell line.

82. The use of any one of claims 79-81, wherein the HSV amplicon particle comprises an HSV-1 or an HSV-2 amplicon particle.

83. The use of any one of claims 80-82, wherein the amplicon vector comprises an origin of replication (ori) and a packaging signal (pac).

84. The use of any one of claims 80-83, wherein the amplicon vector further comprises at least one cargo nucleic acid sequence.

85. The use of claim 84, wherein the at least one cargo nucleic acid sequence comprises a transgene.

86. The use of claim 85, wherein the transgene comprises CRISPR associated protein 9 (Cas9), Cas3, Cas12a, Dystrophin (DYS), amylo-alpha-1, 6-glucosidase, 4-alpha-glucanotransferase (AGL), otoferlin (OTOF), myosin VIIA (MYO7A), ATP binding cassette subfamily A member 4 (ABCA4), coagulation factor VIII (F8), centrosomal protein 290 (CEP290), cadherin related 23 (CDH23), or centrosome and basal body associated protein (ALMS1).

87. The use of claim 85, wherein the transgene comprises a gene encoding an agent for treating a cancer, muscular dystrophy, non-syndromic deafness, Stargardt Disease, Leber congenital amaurosis 10, Alstrom syndrome, a glycogen storage disease, Usher Syndrome 1D, Usher Syndrome 1B, a cardiovascular disease, a neurodegenerative disease, chronic pain, a lung disease, or hemophilia.

88. The use of any one of claims 84-87, wherein the at least one cargo nucleic acid sequence further comprises at least one regulatory element comprising an enhancer, a promoter, a terminator, or a combination thereof.

89. The use of any one of claims 80-88, wherein the amplicon vector comprises a sequence with at least 50% sequence identity to SEQ ID NO: 1, SEQ ID NO: 11, SEQ ID NO: 12, SEQ ID NO: 13, or SEQ ID NO: 14.

90. The use of any one of claims 80-89, wherein the amplicon vector comprises a sequence comprising SEQ ID NO: 1, SEQ ID NO: 11, SEQ ID NO: 12, SEQ ID NO: 13, or SEQ ID NO: 14.

91. A use of a Herpes Simplex Virus (HSV) amplicon particle produced by the method of any one of claims 45-55, the HSV amplicon particle of claim 56 or 57, or the pharmaceutical composition of claim 58 for treating a disease or condition in a subject.

92. The use of claim 91, wherein the disease or condition comprises a cancer, muscular dystrophy, non-syndromic deafness, Stargardt Disease, Leber congenital amaurosis 10, Alstrom syndrome, a glycogen storage disease, Usher Syndrome 1D, Usher Syndrome 1B, a cardiovascular disease, a neurodegenerative disease, chronic pain, a lung disease, or hemophilia.

93. The use of claim 91, wherein the disease or condition comprises a disease or condition caused by a genetic mutation.

94. The use of claim 92, wherein the disease or condition is a cancer.

95. The use of any one of claims 91-94, wherein the subject is a human.

96. A composition comprising a recombinant genome, wherein the recombinant genome comprises:
a. a first nucleic acid sequence comprising one or more Herpes Simplex Virus (HSV) Class I genes; and
b. a second nucleic acid sequence comprising one or more HSV Class I and Class 11 genes.

97. The composition of claim 96, wherein the first nucleic acid sequence further comprises a first one or more regulatory elements.

98. The composition of claim 96 or 97, wherein the first nucleic acid sequence is in a first genomic safe harbor site of the recombinant genome.

99. The composition of claim 98, wherein the first genomic safe harbor site comprises a Rosa26 homolog locus.

100. The composition of any one of claims 96-99, wherein the one or more HSV Class I genes comprise Infected-Cell Polypeptide 4 (ICP4), ICP27, ICP0, ICP34.5, VP16, or a combination thereof.

101. The composition of any one of claims 97-100, wherein the first one or more regulatory elements comprise an enhancer, a promoter, a terminator, or a combination thereof.

102. The composition of claim 101, wherein the first one or more regulatory elements comprise the promoter.

103. The composition of claim 102, wherein the promoter comprises an inducible promoter.

104. The composition of claim 103, wherein the inducible promoter comprises a tetracycline-inducible promoter or a doxycycline-inducible promoter.

105. The composition of claim 103 or 104, wherein the inducible promoter comprises Tet-On promoter comprising TRE3G or TRE-tight.

106. The composition of any one of claims 96-105, wherein the second nucleic acid sequence is in a second genomic safe harbor site of the recombinant genome.

107. The composition of claim 106, wherein the second genomic safe harbor comprises an adeno-associated virus site 1 (AAVS1) locus.

108. The composition of any one of claims 96-107, wherein the one or more HSV Class II genes comprise UL1, UL2, UL3, UL4, UL5, UL6, UL7, UL8, UL9, UL10, UL 11, UL12, UL13, UL14, UL15, UL16, UL17, UL18, UL19, UL20, UL21, UL22, UL23, UL24, UL25, UL26, UL26.5, UL27, UL28, UL29, UL30, UL31, UL32, UL33, UL34, UL35, UL36, UL37, UL38, UL39, UL40, UL41, UL42, UL43, UL44, UL45, UL46, UL47, UL49, UL49A, UL50, UL51, UL52, UL53, UL55, UL56, US1, US2, US3, US4, US5, US6, US7, US8, US9, US10, US11, US12, or a combination thereof.

109. The composition of any one of claims 96-108, wherein the second nucleic acid sequence further comprises a second one or more regulatory elements.

110. The composition of claim 109, wherein the second one or more regulatory elements comprise an enhancer, a promoter, a terminator, or a combination thereof.

111. The composition of claim 110, wherein the second one or more regulatory elements comprise the promoter.

112. The composition of claim 111, wherein the promoter comprises a natural promoter for HSV Class II genes comprising UL1, UL2, UL3, UL4, UL5, UL6, UL7, UL8, UL9, UL10, UL11, UL12, UL13, UL14, UL15, UL16, UL17, UL18, UL19, UL20, UL21, UL22, UL23, UL24, UL25, UL26, UL26.5, UL27, UL28, UL29, UL30, UL31, UL32, UL33, UL34, UL35, UL36, UL37, UL38, UL39, UL40, UL41, UL42, UL43, UL44, UL45, UL46, UL47, UL49, UL49A, UL50, UL51, UL52, UL53, UL55, UL56, US1, US2, US3, US4, US5, US6, US7, US8, US9, US10, US11, US12, or a combination thereof.

113. The composition of any one of claims 96-112, wherein the recombinant genome does not comprise a nucleic acid sequence comprising an HSV origin of replication (ori), an HSV packaging signal (pac), or a combination thereof.

114. The composition of any one of claims 96-113, wherein the recombinant genome does not comprise a nucleic acid sequence comprising oriL/S.

115. The composition of any one of claims 96-114, wherein the first nucleic acid sequence comprises a sequence with at least 50% sequence identity to SEQ ID NO: 2, SEQ ID NO: 3, SEQ ID NO: 7, or SEQ ID NO: 8.

116. The composition of any one of claims 96-115, wherein the first nucleic acid sequence comprises a sequence comprising SEQ ID NO: 2, SEQ ID NO: 3, SEQ ID NO: 7, or SEQ ID NO: 8.

117. The composition of any one of claims 96-116, wherein the second nucleic acid sequence comprises a sequence with at least 50% sequence identity to SEQ ID NO: 5, SEQ ID NO: 6, SEQ ID NO: 9, SEQ ID NO: 10, or SEQ ID NO: 17.

118. The composition of any one of claims 96-117, wherein the second nucleic acid sequence comprises a sequence comprising SEQ ID NO: 5, SEQ ID NO: 6, SEQ ID NO: 9, SEQ ID NO: 10, or SEQ ID NO: 17.

119. A cell or a cell line comprising the composition of any one of claims 96-118.

120. The cell or the cell line of claim 119, wherein the cell or the cell line comprises a mammalian cell or a mammalian cell line.

121. The cell or the cell line of claim 120, wherein the cell or the cell line comprises a Human Bone Osteosarcoma Epithelial (U2OS) cell, a Baby Hamster Kidney (BHK) cell, a Human Embryonic Kidney (HEK 293) cell, or an African Green Monkey Kidney (Vero) cell.

122. The cell or the cell line of claim 121, wherein the U2OS cell comprises an U2OS cell comprising ICP4 and ICP27 genes (U2OS-4/27).

123. The cell or the cell line of claim 121, wherein the U2OS cell comprises an U2OS cell comprising ICP0, ICP4, and ICP27 genes (U2OS-0-4/27).

124. A kit comprising the composition of any one of claims 96-118, or the cell or the cell line of any one of claims 119-123 and an instruction manual for using the kit.

125. A use of the composition of any one of claims 96-118 for producing a packaging cell line.

126. A method of producing a packaging cell line comprising introducing the composition of any one of claims 96-118 into a cell.

127. A method of producing a packaging cell line comprising introducing into a cell comprising a genome:
a. a first nucleic acid sequence comprising one or more Herpes Simplex Virus (HSV) Class I genes; and
b. a second nucleic acid sequence comprising one or more HSV Class I and Class II genes.

128. The method of claim 126 or 127, wherein the cell comprises a mammalian cell.

129. The method of any one of claims 126-128, wherein the mammalian cell comprises a Human Bone Osteosarcoma Epithelial (U2OS) cell, a Baby Hamster Kidney (BHK) cell, a Human Embryonic Kidney (HEK 293) cell, or an African Green Monkey Kidney (Vero) cell.

130. The method of claim 129, wherein the U2OS cell comprises an U2OS cell comprising ICP4 and ICP27 genes (U2OS-4/27).

131. The method of claim 129, wherein the U2OS cell comprises an U2OS cell comprising ICP0, ICP4, and ICP27 genes (U2OS-0-4/27).

132. The method of any one of claims 126-131, wherein the first nucleic acid sequence is introduced into a first genomic safe harbor site of the genome.

133. The method of claim 132, wherein the first genomic safe harbor site comprises a Rosa26 homolog locus.

134. The method of any one of claims 126-133, wherein the second nucleic acid sequence is introduced into a second genomic safe harbor site of the genome.

135. The method of claim 134, wherein the second genomic safe harbor comprises an adeno-associated virus site 1 (AAVS1) locus.

136. The method of any one of claims 126-135, wherein the introducing comprises (i) a recombination mediated cassette exchange; (ii) a homology directed repair; or (iii) a random integration.

137. The method of claim 136, wherein (ii) the homology directed repair comprises (a) a homology directed repair following a targeted double strand break generated by an endonuclease or (b) a homology directed repair with long homology arms and naturally occurring double strand breaks.

138. The method of claim 137, wherein the endonuclease comprises a Clustered regularly interspaced short palindromic repeats (CRISPR)-CRISPR-associated protein 9 (Cas9), a zinc finger nuclease, or a transcription activator-like effector nuclease (TALEN).

139. The method of claim 137, wherein (b) the homology directed repair with long homology arms and naturally occurring double strand breaks further comprises selection with a marker.

140. The method of claim 136, wherein (iii) the random integration further comprises a selection with a marker.

141. The method of claim 139 or 140, wherein the marker comprises LacZ, CAT, HPRT1, PIGA, Luciferase, GFP, YFP, RFP, iRFP, EGFP, blasticidin, neomycin/G418, hygromycin, puromycin, or zeocin.

142. The method of any one of claims 127-141, wherein the first nucleic acid sequence further comprises a first one or more regulatory elements.

143. The method of any one of claims 127-142, wherein the one or more HSV Class I genes comprise Infected-Cell Polypeptide 4 (ICP4), ICP27, ICP0, ICP34.5, VP16, or a combination thereof.

144. The method of any one of claims 127-143, wherein the first one or more regulatory elements comprise an enhancer, a promoter, a terminator, or a combination thereof.

145. The method of claim 144, wherein the first one or more regulatory elements comprise the promoter.

146. The method of claim 145, wherein the promoter comprises an inducible promoter.

147. The method of claim 146, wherein the inducible promoter comprises a tetracycline-inducible promoter or a doxycycline-inducible promoter.

148. The method of claim 146 or 147, wherein the inducible promoter comprises Tet-On promoter comprising TRE3G or TRE-tight.

149. The method of any one of claims 127-148, wherein the one or more HSV Class II genes comprise UL 1, UL2, UL3, UL4, UL5, UL6, UL7, UL8, UL9, UL10, UL 11, UL 12, UL13, UL14, UL15, UL16, UL17, UL18, UL19, UL20, UL21, UL22, UL23, UL24, UL25, UL26, UL26.5, UL27, UL28, UL29, UL30, UL31, UL32, UL33, UL34, UL35, UL36, UL37, UL38, UL39, UL40, UL41, UL42, UL43, UL44, UL45, UL46, UL47, UL49, UL49A, UL50, UL51, UL52, UL53, UL55, UL56, US1, US2, US3, US4, US5, US6, US7, US8, US9, US10, US11, US12, or a combination thereof.

150. The method of any one of claims 127-149, wherein the second nucleic acid sequence further comprises a second one or more regulatory elements.

151. The method of claim 150, wherein the second one or more regulatory elements comprise an enhancer, a promoter, a terminator, or a combination thereof.

152. The method of claim 151, wherein the second one or more regulatory elements comprise the promoter.

153. The method of claim 152, wherein the promoter comprises a natural promoter for HSV Class II genes comprising UL1, UL2, UL3, UL4, UL5, UL6, UL7, UL8, UL9, UL10, UL11, UL12, UL13, UL14, UL15, UL16, UL17, UL18, UL19, UL20, UL21, UL22, UL23, UL24, UL25, UL26, UL26.5, UL27, UL28, UL29, UL30, UL31, UL32, UL33, UL34, UL35, UL36, UL37, UL38, UL39, UL40, UL41, UL42, UL43, UL44, UL45, UL46, UL47, UL49, UL49A, UL50, UL51, UL52, UL53, UL55, UL56, US1, US2, US3, US4, US5, US6, US7, US8, US9, US10, US11, US12, or a combination thereof.

154. The method of any one of claims 127-153, wherein the genome does not comprise a nucleic acid sequence comprising an HSV origin of replication (ori), an HSV packaging signal (pac), or a combination thereof.

155. The method of any one of claims 127-154, wherein the genome does not comprise a nucleic acid sequence comprising oriL/S.

156. The method of any one of claims 126-155, wherein the first nucleic acid sequence and/or the second nucleic acid sequence is comprised in a vector.

157. The method of claim 156, wherein the vector is selected from the group consisting of a DNA, an RNA, a plasmid, a lentivirus vector, adenoviral vector, a Rous sarcoma viral (RSV) vector, and a retrovirus vector.
